(19) United States
(12) Patent Application Publication
(10) Pub. No.: US 2022/0387005 A1
(43) Pub. Date: Dec. 8, 2022

(54) SAMPLE COLLECTION SYSTEM

(71) Applicant: DermTech, Inc., La Jolla, CA (US)

(72) Inventors: John Daniel DOBAK, III, La Jolla, CA (US); Zuxu YAO, San Diego, CA (US); Claudia IBARRA, San Diego, CA (US); Michael HOWELL, La Jolla, CA (US); Jelena OSTOJIC, La Jolla, CA (US); James ROCK, La Jolla, CA (US); Kamil Bugra TOGA, Ann Arbor, MI (US); Hyeonsoo KIM, Ann Arbor, MI (US)

(21) Appl. No.: 17/832,394

(22) Filed: Jun. 3, 2022

Related U.S. Application Data
(60) Provisional application No. 63/197,212, filed on Jun. 4, 2021.
(60) Provisional application No. 63/285,328, filed on Dec. 2, 2021.
(60) Provisional application No. 63/322,968, filed on Mar. 23, 2022.

(51) Int. Cl. A61B 10/02; C12Q 1/6886

(52) U.S. Cl. CPC A61B 10/02; C12Q 1/6886; C12Q 2600/158; A61B 2560/0412

(57) ABSTRACT

The subject matter described herein provides non-invasive patch stripping methods for the collection of a skin sample. The patch stripping method includes applying and removing at least one adhesive patch, provided that a skin sample is adhered to the adhesive patch after removal. The at least one adhesive patch is supplied in an adhesive skin sample collection kit. Optionally, the adhesive skin sample collection kit further comprises a sample collector and/or instructions for use.

### CROSS-REFERENCE

This application claims the benefit of U.S. Provisional Application Ser. No. 63/197,212, filed on Jun. 4, 2021; the benefit of U.S. Provisional Application Ser. No. 63/285,328, filed on Dec. 2, 2021; and the benefit of U.S. Provisional Application Ser. No. 63/322,968, filed on Mar. 23, 2022, each of which is incorporated by reference in their entirety for all purposes.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Skin diseases are some of the most common human illnesses and represent an important global burden in healthcare. Three skin diseases are in the top ten most prevalent diseases worldwide, and eight fall into the top 50. When considered collectively, skin conditions range from being the second to the 11th leading causes of years lived with disability. There remains an unmet need for products and processes that non-invasively, effectively, and efficiently collect skin cells or samples for further analysis of such skin related diseases and conditions.

### BRIEF SUMMARY

Provided herein are systems for non-invasive collection and analysis of a skin sample. Provided herein are systems for non-invasive collection and analysis of a skin sample, the system comprising: an adhesive skin sample collection kit comprising at least one adhesive patch, wherein the least one adhesive patch comprises: a backing layer comprising a collection area; a non-adhesive handling area; and an adhesive matrix on a surface of the collection area, wherein the adhesive matrix is configured to adhere an amount of a skin sample. Further provided herein are systems wherein one or more of the following: (a) the backing layer comprises a flexibility to conform to a morphology of a portion of skin with or without a lesion, and wherein the backing layer comprises a thickness such the at least one adhesive patch resists wrinkling when the at least one adhesive patch is released from the skin; (b) the at least one patch comprises a thickness such that it does not self-adhere when supported by a portion of the non-adhesive handling layer with a draft and in multiple orientations; (c) an amount of extractables and leachables released from the at least one adhesive patch is less about than 3.0 mg/cm² when at least about 25 cm² patch is refluxed for about 3 hours in 80% ethanol; (d) the at least one adhesive patch comprises a longest dimension of about a wrinkling wavelength of the at least one adhesive patch; and/or (e) the adhesive matrix comprises a pressure sensitive adhesive, wherein the pressure sensitive adhesive exhibits a glass transition temperatures lower than 5° C. Further provided herein are systems wherein 2 or more, 3 or more, 4 or more, or 5 or more of (a), (b), (c), (d), and (e). Further provided herein are systems wherein at least (a). Further provided herein are systems wherein the backing layer has an elastic modulus from about 200 to about 2,000 Psi as measured by ASTM D-882. Further provided herein are systems wherein the backing layer has an elastic modulus of from about 1000 to about 2000 Psi. Further provided herein are systems wherein the backing layer has an elastic modulus of from about 500 to about 1500 Psi. Further provided herein are systems wherein the backing layer has a tensile strength of from about 7 to about 60 MPa. Further provided herein are systems wherein the backing layer has a tensile strength of from about 30 to about 60 MPa. Further provided herein are systems wherein the backing layer has a tensile strength of from about 7 to about 15 MPa. Further provided herein are systems wherein at least (b). Further provided herein are systems wherein a thickness of the backing layer is greater than about 2 mil as measured by ASTM D6988. Further provided herein are systems wherein a thickness of the backing layer is from about 3 to about 5 mil. Further provided herein are systems wherein at least (c). Further provided herein are systems wherein the amount of extractables and leachables released from the at least one adhesive patch is less about than 1.0 mg/cm². Further provided herein are systems wherein the amount of extractables and leachables is characterized by GC-MS. Further provided herein are systems wherein the amount of extractables and leachables is characterized by thermogravimetric analysis. Further provided herein are systems wherein an extractable or a leachable comprises a component of the system that is not the skin sample. Further provided herein are systems wherein the extractable or the leachable comprises a non-volatile material, a semi-volatile material, or ash. Further provided herein are systems wherein the adhesive matrix comprises a polymer and wherein the non-volatile material comprises on or more monomers of the polymer. Further provided herein are systems the semi-volatile material comprises a plasticizer or a process aid. Further provided herein are systems wherein an extractable or a leachable comprises BHT and wherein the BHT is less than about 10 ug/L measured by GC-MS. Further provided herein are systems wherein at least (d). Further provided herein are systems wherein the longest dimension is as less than about 10, about 8, about 6, about 5, about 4, or about 3 cm. Further provided herein are systems wherein at least (e). Further provided herein are systems wherein the glass transition temperatures is from about −10 to about −70° C. as measured by ASTM D3418. Further provided herein are systems further comprising a release panel. Further provided herein are systems further comprising at least one placement area panels. Further provided herein are systems wherein the at least one adhesive patch comprises a color. Further provided herein are systems wherein the color of the at least one adhesive patch corresponds to a placement location. Further provided herein are systems comprising at least two adhesive patches, wherein the at least two adhesive patches comprise different colors. Further provided herein are systems comprising at least two adhesive patches, wherein the at least two adhesive patches comprise the same color. Further provided herein are systems wherein the amount of the skin sample is less than about 20 milligrams, less than about 4 milligrams, or from about 1 picogram to about 2000 micrograms of cellular material. Further provided herein are systems wherein an amount of the skin sample on each of the at least one adhesive patch is from about 1 picogram to about 500 micrograms per patch. Further provided herein are systems comprising a plurality of adhesive patches comprising a total amount of the skin sample, wherein the total amount is less than about 20 milligrams, about 10 milligrams, or about 5 milligrams. Further provided herein are systems wherein the adhesive matrix comprises a peel adhesion strength from about 1 to about 30N/inch, as measured by ASTM D3330 at a 180° peel adhesion at a pull rates from about 1.0 inch/min to about 12.0 inch/min. Further provided herein are systems wherein the peel adhesion is from about 10 to about 20 N/inch. Further provided herein are systems wherein the adhesive matrix comprises one or more of an acrylic, a silicone, and a hydrocarbon rubber. Further provided herein are systems wherein the adhesive matrix comprises an acrylic and a hydrocarbon rubber. Further provided herein are systems wherein the acrylic comprises one or more of styrene, α-methyl styrene, vinyl naphthalene, vinyl toluene, chloromethyl styrene, methyl acrylate, acrylic acid, methacrylic acid, methyl methacrylate, ethyl acrylate, ethyl methacrylate, butyl acrylate, butyl methacrylate, isobutyl acrylate, isobutyl methacrylate, ethylhexyl acrylate, ethylhexyl methacrylate, lauryl methacrylate, lauryl acrylate, octyl acrylate, octyl methacrylate, glycidyl methacrylate, allyl methacrylate, vinyl methacrylate, acetoacetoxyethyl acrylate, acetoacetoxyethyl methacrylate, acetoacetoxypropyl acrylate, acetoacetoxypropyl methacrylate, hydroxybutenyl methacrylate, the allyl ester of maleic acid, the diallyl ester of maleic acid, poly(allyl glycidyl ether), alkyl crotonates, vinyl acetate, di-n-butyl maleate, di-octylmaleate, acrylonitrile, diacetone acrylamide, acrylamide, methacrylamide, hydroxyethyl methacrylate, hydroxyethyl acrylate, acrylonitrile, t-butylaminoethyl methacrylate, dimethylaminoethyl methacrylate, diethylaminoethyl methacrylate, N,N-dimethylaminopropyl methacrylamide, 2-t-butylaminoethyl methacrylate, N,N-dimethylaminoethyl acrylate, N-(2-methacryloyloxy-ethyl)ethylene urea, and methacrylamidoethylethylene urea, or combinations thereof. Further provided herein are systems wherein the hydrocarbon rubber comprises one or more of butyl rubber, styrene-butadiene rubber, ethyl-vinyl acetate polymers, styrene-isoprene-butadiene rubbers, or combinations thereof. Further provided herein are systems wherein the backing layer comprises a soft, clear or transparent, and/or pliable synthetic polymer. Further provided herein are systems wherein the synthetic polymer comprises a thermoplastic polyurethane (TPU) or low density polyethylene (LDPE). Further provided herein are systems wherein the synthetic polymer comprises polyethylene terephthalate (PET), Teflon, polyimide, polyethylene naphthalate (PEN), or acetate. Further provided herein are systems wherein the synthetic polymer comprises an elastomer of olefin. Further provided herein are systems wherein the elastomer of olefin comprises copolymers or compounds of polymers comprising one or more of ethylene, propylene, isobutylene, vinyl acetate, vinyl alcohol, ethylene oxide, and propylene oxide. Further provided herein are systems wherein the soft clear or transparent, and/or pliable synthetic polymer comprises a thermoplastic elastomer. Further provided herein are systems wherein the thermoplastic elastomer comprises a polyester based elastomer. Further provided herein are systems wherein the thermoplastic elastomer comprises a copolymer or compound of an ether or an amide. Further provided herein are systems wherein the at least one adhesive patch has a haze value less than about 30% as measured by ASTM D1003. Further provided herein are systems wherein the haze value is less than about 15%. Further provided herein are systems wherein at least one of the backing layer and adhesive matrix is water soluble. Further provided herein are systems wherein the at least one adhesive patch is water soluble. Further provided herein are systems wherein at least one of the backing layer and adhesive matrix is configured to dissolve during skin sample lysis. In various embodiments, both the backing layer and adhesive matrix are water soluble. Further provided herein are systems wherein the adhesive matrix described herein comprises at least 12 oz/in² loop tackiness. Further provided herein are systems wherein the adhesive matrix comprises a working temperature range from −40 to 176° F. Further provided herein are systems wherein backing layer comprises at least 20 lb/inch tensile force. Further provided herein are systems wherein backing layer comprises at least 200 mN tear strength. Further provided herein are systems wherein the adhesive patch is dissolvable, such as in a liquid or solvent, within no more than 30 seconds. Further provided herein are systems wherein the adhesive patch is dissolvable in an aqueous solution within no more than 30 seconds. Further provided herein are systems wherein the adhesive patch is dissolvable, such as in a liquid or solvent, within no more than 30 seconds at 30-80 degrees C. Further provided herein are systems wherein the adhesive patch is dissolvable in an aqueous solution within no more than 30 seconds at 30-80 degrees C. Further provided herein are systems wherein the adhesive patch has a shelf life of at least 12 months.

Provided herein are kits comprising a system described herein and further comprising a packaging component comprising instructions to perform one or more of the following: place the patch or patches on one or more specified areas of the body; demarcate a region surrounding a lesion on a skin; peel the patch slowly; and peel the patch at an angle greater than about perpendicular to the skin surface. Further provided herein are kits wherein peeling slowly is indicated to be as less than about 1 linear inch peeled per about five seconds.

Provided herein are kits comprising: at least one adhesive patch, wherein the least one adhesive patch comprises: a backing layer comprising a collection area; a non-adhesive handling area; an adhesive matrix on a surface of the collection area, wherein the adhesive matrix is configured to adhere to an amount of a skin sample; and a packaging comprising instructions to perform one or more of the following: apply the at least one patch to a specific part of the body (e.g., to the face, such as on the forehead, cheek and/or chin); demarcate a region surrounding a lesion on a skin; peel the patch slowly; and peel the patch at an angle greater than about perpendicular to the skin surface. Further provided herein are kits wherein slowly is indicated as less than about 1 linear inch peeled per about five seconds. Further provided herein are kits wherein one or more of the following: (a) the backing layer comprises a flexibility to conform to a morphology of a portion of skin, and wherein the backing layer comprises a thickness such the at least one adhesive patch resists wrinkling when the at least one adhesive patch is released from the skin; (b) the at least one patch comprises a thickness such that it does not self-adhere when supported by a portion of the non-adhesive handling layer with a draft and in multiple orientations; (c) an amount of extractables and leachables released from the at least one adhesive patch is less about than 3.0 mg/cm² when at least about 25 cm² patch is refluxed for about 3 hours in 80% ethanol; (d) the at least one adhesive patch comprises a longest dimension of about a wrinkling wavelength of the at least one adhesive patch; and/or (e) the adhesive matrix comprises a pressure sensitive adhesive, wherein the pressure sensitive adhesive exhibits a glass transition temperatures lower than 5° C. Further provided herein are kits wherein 2 or more, 3 or more, 4 or more, or 5 or more of (a), (b), (c), (d), and (e). Further provided herein are kits wherein the portion of skin comprises a lesion. Further provided herein are kits wherein the portion of skin comprises non-lesional skin. Further provided herein are kits wherein the portion of skin comprises normal skin.

Provided herein are kits for non-invasive collection and analysis of a skin sample, the kit comprising: at least one adhesive patch, wherein the least one adhesive patch comprises: a backing layer comprising a collection area; a non-adhesive handling area; an adhesive matrix on a surface of the collection area, wherein the adhesive matrix is configured to adhere to an amount of a skin sample. In some embodiments, the kit comprises at least two (2) to sixteen (16) adhesive patches, e.g., at least 2 adhesive patches, at least 4 adhesive patches, at least 8 adhesive patches, at least 12 adhesive patches, at least 14 adhesive patches, at least 16 adhesive patches, or any number of patches in between. In some embodiments, the kit further comprises a return or storage receptacle sized and shaped to receive the at least one adhesive patch. In some embodiments, the return or storage receptacle comprises a desiccant. Further provided herein are kits wherein the desiccant is configured to prevent the activity of RNases in the skin sample adhered to the at least one adhesive patch. Further provided herein are kits wherein the desiccant is configured to prevent the activity of DNases in the skin sample adhered to the at least one adhesive patch. Further provided herein are kits wherein the desiccant is configured to prevent the activity of proteases in the skin sample adhered to the at least one adhesive patch. Further provided herein are kits wherein an amount of the desiccant is from about 0.5 grams to about 5 grams. Further provided herein are kits wherein the amount of the desiccant is about 2 grams. Further provided herein are kits wherein the return or storage receptacle comprises a bag, pouch, or tube. Further provided herein are kits wherein the return receptacle is plastic or foil. Further provided herein are kits wherein the return receptacle is sealable. Further provided herein are kits wherein the desiccant is silica gel. Further provided herein are kits further comprising a packaging comprising instructions to perform one or more of the following: apply the at least one patch to a specific part of the body (e.g., to the face, such as on the forehead, cheek and/or chin); demarcate a region surrounding a lesion on a skin; peel the patch slowly; and peel the patch at an angle greater than about perpendicular to the skin surface. Further provided herein are kits wherein slowly is indicated as less than about 1 linear inch peeled per about five seconds.

Provided herein are methods for analyzing a skin sample comprising: receiving at least one adhesive patch from the system or kit described herein; and quantifying expression levels of one or more target analyte in the skin sample. In some embodiments, the target analyte is a gene. In other embodiments, the target analyte is a protein. Further provided herein are methods wherein the method further comprises extracting nucleic acids from at least a portion of the skin sample. Further provided herein are methods wherein the skin sample comprises a lesion. Further provided herein are methods wherein the skin sample comprises non-lesional skin. Further provided herein are methods wherein the skin sample comprises normal skin. Further provided herein are methods wherein the target analyte is a RNA or DNA molecule. Further provided herein are methods wherein the target analyte is a protein or polypeptide molecule. Further provided herein are methods wherein quantifying one or more target analytes in the skin sample comprises measuring expression levels. Further provided herein are methods wherein the method further comprises extracting nucleic acids from at least a portion of the skin sample. Further provided herein are methods wherein the one or more target analytes are of human and/or microbial origin. Further provided herein are methods wherein the at least one adhesive patch comprises a color. Further provided herein are methods wherein the color of the at least one adhesive patch corresponds to a placement location. Further provided herein are methods comprising at least two adhesive patches, wherein the at least two adhesive patches comprise different colors. Further provided herein are methods comprising at least two adhesive patches, wherein the at least two adhesive patches comprise the same color.

Further provided herein are methods wherein the at least one adhesive patch is applied to a single placement location. Further provided herein are methods wherein the at least one adhesive patch is applied to two or more placement locations. Further provided herein are methods wherein the at least one adhesive patch is applied once to each placement location. Further provided herein are methods wherein the at least one adhesive patch is applied two or more times to each placement location. Further provided herein are methods wherein the method comprises use of at least 2, 4, 8, or at least 12 adhesive patches. Further provided herein are methods wherein quantifying one or more target analytes in the skin sample comprises detecting at least one nucleic acid mutation. Further provided herein are methods wherein the sample comprises a majority of skin sampled from a layer of skin exposed to an environmental factor. Further provided herein are methods wherein the environmental factor is ultraviolet (UV) light. Further provided herein are methods wherein the number of nucleic acid mutations per mm² of skin collected comprises at least 10 mutations. Further provided herein are methods wherein the at least one nucleic acid mutation is indicative of UV damage. Further provided herein are methods wherein analyzing comprises identifying a disease or condition. Further provided herein are methods wherein the disease or condition comprises an autoimmune/inflammatory disease. Further provided herein are methods wherein the autoimmune/inflammatory disease comprises atopic dermatitis, psoriasis, or lupus. Further provided herein are methods wherein the disease or condition comprises an proliferative disease. Further provided herein are methods wherein proliferative disease comprises melanoma, actinic keratosis, basal cell carcinoma, squamous cell carcinoma, or cutaneous T-cell lymphoma.

### BRIEF DESCRIPTION OF THE DRAWINGS

The novel and inventive features of the subject matter described herein are set forth with particularity in the appended claims. A better understanding of the feature and advantages of the present invention will be obtained by reference to the following detailed description that sets forth illustrative embodiments, in which the principles of the invention are utilized, and the accompanying drawings of which:

FIG. 1 illustrates cleansing a skin sampling area comprising a skin lesion.

FIG. 2 illustrates a tri-fold skin sample collector comprising a peelable release panel comprising four adhesive patches, a placement area panel comprising a removable liner, and a clear or transparent panel.

FIG. 3 illustrates removing a first adhesive patch positioned at the far left side of a peelable release panel of a tri-fold skin sample collector.

FIG. 4 illustrates an adhesive patch positioned on a cleansed skin sampling area comprising a skin lesion.

FIG. 5 illustrates pressing firmly on an adhesive patch positioned on a cleansed skin sampling area while making a circular motion.

FIG. 6 illustrates demarcating a region comprising a skin lesion on an adhesive patch.

FIG. 7 illustrates placing a used adhesive patch comprising a skin sample onto a placement area panel of a tri-fold skin sample collector.

FIG. 8 illustrates an adhesive skin sample collection kit.

FIG. 9A illustrates storage of patches comprising nucleic acids stored in bags with or without desiccant.

FIG. 9B illustrates storage of patches comprising nucleic acids stored in bags with or without desiccant, wherein each sample was split prior to storage.

FIG. 10A illustrates a graph of total RNA yields isolated from the dried cells on adhesive patches stored for 2 days in different conditions, including that stored at −80° C. (To Frozen), in humidity chamber without desiccant, and with 1, 4, and 10 desiccant pouches, and from patches stored in sealable plastic bags (no hatching) or in foil bags (hatched bars), all after a 2 day (48 hours) storage.

FIG. 10B illustrates a graph of % change to compared to time zero vs. different storage conditions for 48 hours.

FIG. 11 illustrates a graph of percentage (fold) of RNA yield change from samples stored in foil bags with 4 desiccant pouches (squares) and without desiccant pouch (diamonds, control) in a humid chamber (70% humidity) for 2, 10 and 20 days, compared to the RNA yields from samples extracted fresh (on day 0).

FIG. 12A illustrates a graph of total RNA yields isolated from skin patches collected from the skin of 12 subjects (human volunteers).

FIG. 12B illustrates a graph of % of RNA yield change between the 2 conditions (stored with and without desiccant), calculated as %=(RNA Yield-with desiccant−RNA Yield Without desiccant)/RNA Yield without desiccant, for each subject.

FIG. 13 illustrates a graph of % of RNA yield gain from patches stored in foil bags with 4 desiccant pouches (per bag), compared to their counterpart stored in bags without desiccant, in humidity chamber for 2, 10 and 20 days.

FIG. 14A illustrates an example of a patch after obtaining a skin sample. The patch has little to no visible wrinkling.

FIG. 14B illustrates an example of a patch after obtaining a skin sample. The patch has visible wrinkling.

FIG. 14C illustrates another example of patch after obtaining a skin sample. The patch has visible wrinkling.

FIG. 15 illustrates exemplary positions of 14 sampling tapes on selected upper back sites.

FIG. 16A illustrates a graph of levels of TEWL and SCH before and after stripping with four consecutive tapes. Bar graphs represent average values for all tapes (N=14).

FIGS. 16B-16F illustrate graphs of performance properties for tapes described herein. D-Squame skin sampling disc (CuDerm Corp, “DSQ” herein) was used as a comparator device (T14). A skin sample collector, such an example, variation, or embodiment as those described in commonly owned International Patent Publication No. WO2016/179043, which is incorporated by reference herein in its entirety, was used as a comparator device (T13 or “CC”). FIG. 16B illustrates post-stripping TEWL values for individual tapes (N=21). Barrier disruption cutoff of 30 g/m²/h is indicated with a horizontal line. D-Squame skin sampling disc (CuDerm Corp, “DSQ” herein) was used as a comparator device (T14). A skin sample collector, such an example, variation, or embodiment as those described in commonly owned International Patent Publication No. WO2016/179043, which is incorporated by reference herein in its entirety, was used as a comparator device (T13 or “CC”). FIG. 16C illustrates a graph of average values for total RNA yields (pg) per tape (N=21). FIG. 16D illustrates a graph of average values for total DNA yields (pg) per tape (N=21). FIG. 16E illustrates QQ plots showing the distribution of RNA (left) and DNA (right) yield values in the 21 subject cohort, compared to a normally distributed population (dotted diagonal line). FIG. 16F illustrates a visual representation of the RNA electropherogram of Subject 7, showing results for tapes 5-14. Intensity of the bands correlates with yield. Subject 7 displayed higher than average RNA integrity.

FIG. 16G illustrates a bar graph showing differences in average yields between different subjects. Group one-way ANOVA (non-parametric Kruskall-Wallis test) yields a p-value of <0.0001.

FIG. 17A illustrates an overlaid GC-MS chromatogram of 20% ethanol extractions from samples (Circled: Sample 2 distinct peak at around 31 min). The x-axis is labeled 24.50 to 31.00 at 0.5 minute intervals.

FIG. 17B illustrates an overlaid GC-MS chromatogram of 20% ethanol extractions from samples (Circled: Sample 3 at 18.6 min, Sample 10 at 19.6 min and Sample 1 at 21 min). The x-axis is labeled 17.50 to 22.50 at 0.5 minute intervals.

FIG. 17C illustrates an overlaid GC-MS chromatogram of 80% ethanol extractions from samples. Circled: All samples at 10.1 min except for sample 1 (confirmed by individual overlay with Sample 1). Sample 3, Sample 7, Sample 8, and Sample 9 at 10.8 min (confirmed by individual overlay with Sample 1), Sample 5 at 12.8 min and Sample 8 at 14.9 min. The x-axis is labeled 10.00 to 15.00 at 0.5 minute intervals.

FIG. 17D illustrates an overlaid GC-MS chromatogram of 80% ethanol extractions from samples. (Circled: Sample 1 at 16.2 min and 21 min, Sample 2, Sample 3, Sample 4 and Sample 6 at 25.5-26.5 min (confirmed by individual overlay with Sample 1). The x-axis is labeled 16.00 to 26.00 at 0.5 minute intervals.

FIG. 18 illustrates a graph of peel strengths (N/in) as a function of adhesive thickness (mil). The trendline is labeled as y=10.421x0.7216, R²=0.7563.

FIG. 19 illustrates a graph of tack adhesion (cm) as a function of adhesive thickness (mil). The trendline is labeled as y=8.0016x−1.498, R²=0.8828.

FIG. 20 illustrates an example of severe wrinkle formation on Tape 01 placed on the upper arm of Panelist #2.

FIG. 21A illustrates a graph of number of wrinkles as a function of backing sheet thickness (mil).

FIG. 21B illustrates a graph of number of wrinkles as a function of adhesive layer thickness (mil).

FIG. 22A illustrates a graph of discomfort rating as a function of backing sheet thickness (mil).

FIG. 22B illustrates a graph of discomfort rating as a function of adhesive thickness (mil).

FIG. 23 illustrates a graph of enrollment and baseline demographics for a longitudinal pigmented lesion assay study. For each set of bars in the graph, the left bar represents total samples and the right bar represents usable samples.

FIG. 24 shows a comparison of each sample for expression of four different genes LINC00518, ACTB, PRAME, and PPIA as a function of the cycle threshold for tapes T14, T13, T7, and T12. D-Squame skin sampling disc (CuDerm Corp, “DSQ” herein) was also used as a comparator device (T14). A skin sample collector, such an example, variation, or embodiment as those described in commonly owned International Patent Publication No. WO2016/179043, which is incorporated by reference herein in its entirety, was also used as a comparator device (T13 or “CC”).

FIG. 25 shows various tape shapes which may increase a collection area of a sample.

FIG. 26 illustrates a graph of the amount of total protein extracted (mg/mL) from tapes T7 and T12 after skin sampling. Samples were collected from 10 healthy volunteers with four tapes per site.

### DETAILED DESCRIPTION

Provided herein are compositions, devices, methods, and systems for collecting skin samples. Further provided herein are non-invasive stripping methods for the collection of a skin sample. Further provided herein are adhesives, materials, and other components which in some instances result in higher sampling yields, patient comfort, ease of use, and/or other improvement.

### Skin Sample Collector System

Provided herein are systems and devices for collecting skin samples (skin sample collector). In some instances, a skin sample collector (or system) comprises one or more adhesive patches (tapes, stickers, strips, or other collector). In some instances, an adhesive patch comprises one or more of: a backing layer, an adhesive matrix, and a non-invasive handling area. In some instances, a skin sample collector further comprises one or more of a release panel, individual liners, a placement area, and individual panels. In some instances, devices are configured for optimum peel adhesion, elasticity of the backing film, extractables, dimensions, materials, functional results, or a combination thereof. In some instances, the backing layer comprises a flexibility and/or elasticity to conform to a morphology of a portion of skin, and wherein the backing layer comprises a thickness such the at least one adhesive patch resists wrinkling. In some instances, the backing layer comprises a combination of dimensions, flexibility, and/or elasticity such that the at least one adhesive patch resists wrinkling. The wrinkling may be static wrinkling, such as wrinkling when a patch is on the skin. The wrinkling may be dynamic wrinkling, such as wrinkling when the at least one adhesive patch is released from the skin. In some instances, the at least one patch comprises a thickness such that it does not self-adhere when supported by a portion of the non-adhesive handling layer with a draft and in multiple orientations. In some instances, an amount of extractables and leachables released from the at least one adhesive patch is minimized to improve target analyte, such as a nucleic acid, analysis. In some instances, the at least one adhesive patch comprises a longest dimension of about a wrinkling wavelength of the at least one adhesive patch. In some instances, the adhesive matrix comprises a pressure sensitive adhesive, wherein the pressure sensitive adhesive exhibits a glass transition temperatures lower than 5° C. In some instances the portion of skin comprises a lesion (lesional), comprises non-lesional skin, or comprises normal skin.

The adhesive patch of the adhesive skin sample collector typically comprises a backing layer. In some instances, the backing area comprises a first collection area comprising an adhesive matrix and a second area extending from the periphery of the first collection area. The adhesive matrix is located on a skin facing surface of the first collection area. The second area functions as a tab (or non-adhesive handling area), suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use. In some instances, the backing comprises a synthetic polymer. In some instances, the backing comprises a soft, clear or transparent, and/or pliable synthetic polymer.

The backing layer may comprise any material or mixture of materials which controls rigidity or flexibility. Without being bound by theory, a backing layer enables proper conformation of the patch over the lesion of any size or shape, which leads to higher removal of cellular materials during peeling off/collection. In some instances, the thickness or rigidness of the backing layer is configured to prevent deformation due to static wrinkles or slip-stick patterns during peel. In some embodiments, the backing layer comprises a polyurethane carrier film. Patches described herein may comprise any number of materials which provide for the desired sampling properties (e.g., thickness, performance, patient comfort, or other property).

The backing layer may comprise materials or mixtures of materials selected to mitigate wrinkling of the backing layer. Wrinkling of the backing layer may be characterized by a wrinkling pattern. In some cases, the wrinkling pattern may be a regular pattern. The wrinkling pattern may be irregular. A pattern of the wrinkling may be characterized by a wrinkling wavelength (e.g., an average wavelength). The wrinkling wavelength may be a distance (e.g., an average distance) between subsequent peaks or subsequent troughs in the wrinkles. In some instances, wrinkling comprises the average distance between the peak points of the periodic (and standing) wavy structures formed on the skin when a stiffer tape is applied on typically softer skin. An average wavelength may be determined from an average distance between peaks for the length of the tape. Wrinkling may be static or dynamic. Static wrinkling may occur when a backing layer comprising an adhesive is adhered to a surface (e.g., a skin). Dynamic wrinkling may occur during peeling of the backing layer. In some instances, the wrinkling wavelength approaches the length of a patch, such as 50%, 70%, 90%, 95%, 97%, 98%, 99%, 99.5%, or 99.9% of the length of a patch. In some instances, wrinkling is increased with the use of higher flexibility backing layers. In some instances, backing layers of at least 1, 2, 3, 4, 5, 6, or more than 6 mils result in a reduction in wrinkling.

In some cases, dynamic wrinkling may be caused by sticking and slipping of the backing layer during peeling. The process of peeling a backing layer comprising an adhesive may include dynamic sticking and slipping. For example, even when a user endeavors to peel a backing layer as smoothly as possible, the peeling may stop and start causing the effect of sticking and slipping. For example, during a stick, elastic potential energy may be stored in the adhesive and the bend of the tape. In some cases, both the tape and the adhesive may act like springs that store energy as they are stretched. During a slip, potential energy may be converted to kinetic energy. The sticking and slipping may occur even on microscopic length scales (e.g. length scales on the order of few microns or greater). Sticking and slipping may result in defects (e.g., wrinkles) during a peeling step. While not wishing to be bound by theory, the frequency of the stick-slip patterns in some instances decreases with the square root of the patch thickness. For example, the modulus of elasticity of the backing sheet may at least partially govern the wrinkling wavelength by the square root of the cubic root, which provides an exponent of ⅙, (i.e. λ˜Et1/6).

Parameters which effect the sticking and slipping may include elasticity of the skin, elasticity of the backing layer, strength of the adhesive, and geometric parameters such as the length and width of the tape. One or more of these parameters may affect a wavelength and frequency of wrinkling patterns in the backing layer. The skin elasticity may relate to the potential energy stored in a stick. For example, skin with a high elasticity may store greater potential energy during a stick and slip to a greater distance. The elasticity of the backing layer may relate to the potential energy stored in a stick. For example, a backing layer with a high elasticity may store greater potential energy during a stick and slip to a greater distance. The adhesive may relate to the potential energy stored in a stick. For example, a stronger adhesive may store greater potential energy during a stick and slip to a greater distance. In some cases, a separation front, the line dividing the attached portion to the separated portion, may not be a straight line during slips. For example, a slip may propagate along a width of the backing layer if the peel is along a length of the backing layer. Accordingly, a wider tape may change the wrinkling properties of the tape by changing the slip dynamics and/or by increasing the potential energy to peel per unit distance peeled along the peeling axis. In some examples, the wrinkling wavelength may be on the order of several centimeters. A wrinkling wavelength which is longer than the backing layer may mitigate dynamic wrinkles.

Static wrinkling may occur when an adhesive patch is attached to the skin. In some cases, static wrinkling may be caused by a mismatch between the extent of contraction of the soft foundation (e.g., skin) and the harder surface (e.g., the backing layer of the tape) due to the in-plane forces exerted by the adhesive. Parameters which effect the static wrinkling may include elasticity of the skin, elasticity of the backing layer, strength of the adhesive, and geometric parameters such as the length and width of the tape. One or more of these parameters may affect a wavelength and frequency of wrinkling patterns in the backing layer. The extent of contraction of the soft foundation may be related to the elasticity of the soft foundation. The extent of contraction of the harder surface may be related to the elasticity of the backing layer. A mismatch between the extents of contraction may create a deformation in the peel (e.g., a wrinkle). The deformation may be characterized by an amplitude. A mismatch between the extents of contraction may cause static wrinkles. The frequency of static wrinkles may be strongly correlated with the thickness of the backing layer. In some examples, the wrinkling wavelength may be on the order of several centimeters. A wrinkling wavelength which is longer than the backing layer may mitigate static wrinkles. In some cases, a backing layer with a thickness greater than 3 mil or above may provide a wrinkling wavelength of several centimeters.

In some embodiments, the wrinkling wavelength is configured to mitigate static and/or dynamic wrinkling. In some examples, the wrinkling wavelength may be on the order of several centimeters. A wrinkling wavelength that is longer than a length of the backing layer may mitigate wrinkling. A wrinkling wavelength that is longer than a length of a patch applied to the skin may mitigate wrinkling. The wrinkling wavelength may comprise a length which is equal to or greater than, for example and without limitation, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm.

In some instances, patches described herein comprise a backing layer. In some embodiments, the backing layer comprises one or more of TPU (thermoplastic polyurethane), LPDE (low density polyethylene), PET (polyethylene), PP (polypropylene), Teflon, Polyimide, PEN (Polyethylene naphthalate), PVB (polyvinyl butyral), PVOH (poly(vinyl alcohol)), PVP (Poly(vinylpolypyrrolidone)) cellulose butyrate, cellulose acetate, or a mixture thereof. In some embodiments, the backing layer comprises TPU (thermoplastic polyurethane) and LPDE (low density polyethylene). In some instances, the soft, clear or transparent, and pliable synthetic polymer comprises an elastomer of olefin. In some instances, the elastomer of olefin comprises copolymers or compounds of polymers comprising one or more of ethylene, propylene, isobutylene, vinyl acetate, vinyl alcohol, ethylene oxide, and propylene oxide. In some instances, the soft clear or transparent, and pliable synthetic polymer comprises a thermoplastic elastomer. In some instances, the thermoplastic elastomer comprises a polyester based elastomer. In some instances, the thermoplastic elastomer comprises a copolymer or compound of an ether or amide.

In some instances, flexibility is controlled by properties of the backing layer, the adhesive matrix, or both. In some instances, patches are configured to adhere to atypical/3-dimensional morphologies. In some instances, patches comprise a conformability/flexibility to contact the morphological structure of the lesion while minimizing or avoiding wrinkling of the patch upon peel/release. In some instances, flexibility and the thickness of the backing layer provides for the proper conformation of the patch over a part of the body (such as a concave or convex part of the body) and/or a lesion of any size or shape, which leads to higher removal of skin cells during peeling off/collection. In some instances, the size of the is no more than 5, 4, 3, 2, 1.5, 1, 0.8, 0.7, 0.5, 0.3, 0.2, or no more than 0.1 square centimeters. In some instances, flexibility is measured using ASTM D882 or ASTM D1938 methods with an XLW (EC) Auto Tensile Tester (Labthink Instrument Inc). In some instances, the thickness of the backing layer is no more than 7, 6, 5, 4, 3, 2.5, 2.0, 1.5, 1.25, 1, 0.8, 0.7, 0.6, 0.5, 0.3, 0.2, or no more than 0.1 mils. In some instances, the thickness of the backing layer is about 7, 6, 5, 4, 3, 2.5, 2.0, 1.5, 1.25, 1, 0.8, 0.7, 0.6, 0.5, 0.3, 0.2, or about 0.1 mils. In some instances, the thickness of the backing layer is 0.1-5, 0.1-4, 0.1-3, 0.1-2, 0.1-1, 0.5-4, 0.5-3, 1-5, 2-7, 3-5, 3-10, or 1-2 mils. In some instances, a backing layer comprising one or more of LDP or TPU has a thickness of at least 1, 2, 2.5, 3, 3.5, 4, 4.5, 5, 5.5, or more than 6 mils.

In some instances, elasticity is controlled by properties of the backing layer, the adhesive matrix, or both. In some instances, patches are configured to adhere to atypical/3-dimensional morphologies. In some instances, patches comprise an elasticity to contact the morphological structure of the lesion while minimizing or avoiding wrinkling of the patch upon peel/release. The elasticity may be characterized by an elastic modulus. In some instances, the backing layer has an elastic modulus from about 200 to about 2,000 Psi as measured by ASTM D-882. In some instances, the backing layer has an elastic modulus of about 250, 500, 750, 1000, 1250, 1500, 1750, 2000, 2250, 2500, 3000, 3250, 3500 or about 4000 Psi. In some instances, the backing layer has an elastic modulus of from about 1000 to about 2000 Psi, about 500 to about 3000 Psi, about 250 to about 2000 Psi, about 400 to about 2000 Psi, about 500 to about 1500 Psi, about 750 to about 2000 Psi, about 1000 to about 3000 Psi, about 1000 to about 4000 Psi, about 2000 to about 4000 Psi, or about 500 to about 2500 Psi. In some instances, the backing layer has a tensile strength of from about 7 to about 60 MPa, about 5 to about 60 MPa, about 10 to about 60 MPa, about 20 to about 80 MPa, about 30 to about 60 MPa, about 5 to about 30 MPa, about 5 to about 20 MPa, or about 7 to about 15 MPa. In some instances, the backing layer has an elongation of 100-1000%, 100-750%, 100-500%, 150-500%, 200-1000%, 400-600%, 400-800%, 500-1000%, 750-1000%, or 750-1500%. Tensile strength and/or elongation in some instances is measured using CD (cross-direction) or MD (machine direction) test values.

Adhesive patches described herein may comprise an adhesive matrix. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent. In some instances, the adhesive matrix comprises one or more of acrylics, silicones, and hydrocarbon rubbers (like butyl rubber, styrene-butadiene rubber, ethyl-vinyl acetate polymers, styrene-isoprene-butadiene rubbers), or combination thereof. In some instances, tack of the adhesive matrix is measured by ASTM D1876 using XLW (EC) Auto Tensile Tester (Labthink Instrument Inc). In some instances, the adhesive matrix comprises a hydrophobicity of no more than 2000, 1500, 1000, 900, 800, 700, 600, 500, 400, 300, 200, or no more than 150 g/m²/24 hours. In some instances, hydrophobicity is measured as an upright MVTR (moisture vapor transmission rate) or inverted MVTR. In some instances, hydrophobicity is measured using ASTM E96-80. In some instances, the patch (including adhesive matrix) comprises a hydrophobicity of no more than 2000, 1500, 1000, 900, 800, 700, 600, 500, 400, 300, 200, or no more than 150 g/m²/24 hours. In some instances, the adhesive matrix comprises a peel adhesion, or force exerted when removing a patch comprising the adhesive matrix. In some instances, peel adhesion is optimal when the desired amount of cellular material is removed from the skin, but without causing skin damage or discomfort to the patient. In some instances, the peel adhesion is measured using ASTM D3330. In some instances, peel adhesion is measured using PSTC-1. In some instances, the peel adhesion is 1-40, 1-30, 1-20, 5-30, 5-25, 5-20, 5-15, 3-15, 3-12, 10-20, 5-30, 15-30, or 3-10 Newtons/inch. In some instances, the peel adhesion is at least 1, 2, 3, 4, 5, 6, 7, 8, 9, 10, 12, 15, 20, 25, 30, or at least 35 Newtons/inch. In some instances, the peel adhesion is no more than 1, 2, 3, 4, 5, 6, 7, 8, 9, 10, 12, 15, 20, 25, 30, or no more than 35 Newtons/inch. In some instances, the peel adhesion is about 1, 2, 3, 4, 5, 6, 7, 8, 9, 10, 12, 15, 20, 25, 30, or about 35 Newtons/inch. In some instances, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 180° peel adhesion at a pull rates from about 1.0 inch/min to about 12.0 inch/min. In some instances, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 180° peel adhesion at a pull rates from about 4.0 inch/min to about 16.0 inch/min. In some instances, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 180° peel adhesion at a pull rates from about 0.5 inch/min to about 8 inch/min. In some instances, the adhesive matrix comprises a pressure sensitive adhesive. In some instances, the pressure sensitive adhesive exhibits a glass transition temperature lower than 20° C., 15° C., 10° C., 7° C. 6° C., 5° C., 4° C., 3° C., or lower than 2° C. In some instances, the pressure sensitive adhesive exhibits a glass transition temperature of 1-20° C., 1-15° C., 1-10° C., 1-7° C. 3-8° C., 4° C.-6° C. or 4° C.-10° C. In some instances, the pressure sensitive adhesive exhibits a glass transition temperature of about 20° C., 15° C., 10° C., 7° C., 6° C., 5° C., 4° C., 3° C., or about 2° C. In some instances, pressure-sensitive tack of an adhesive is measured. In some instances, pressure-sensitive tack of an adhesive is measured using ASTM D2979. In some instances, pressure-sensitive tack of the adhesive is 100-200, 100-500, 100-750, 100-1000, 150-500, 150-300, 200-500, 200-750, 300-400, 300-600, 450-750, or 500-1000 grams per square inch. In some instances, pressure-sensitive tack of the adhesive is about 50, 75, 100, 125, 150, 175, 200, 250, 300, 350, 400, 500, 600, 700, 800, or about 1000 grams per square inch.

Adhesives may be configured to reduce wrinkling during skin patch sampling. In some instances, provided herein are patches comprising an adhesive. In some instances, provided herein are patches comprising a hybrid adhesive (comprising two or more components). In some instances, adhesives comprise-pressure sensitive adhesives. In some instances, the adhesive comprises a component selected from two or more of silicone, acrylate polymer, or rubber (natural or synthetic). In some instances, acrylic polymer comprises “pure” acrylic or modified acrylic adhesives. In some instances, synthetic rubber comprises hot-melt rubber, solvent rubber, or butyl rubber. In some instances, the adhesive comprises one or more components. In some instances, the adhesive comprises a first component, wherein the first component comprises a synthetic rubber adhesive. In some instances, the adhesive comprises a second component, wherein the second component comprises an acrylate polymer. In some instances, the adhesive is applied to patch comprising a polyester backing layer. Without being bound by theory, using a hybrid adhesive which rapidly reaches its maximal adhesion in some instances reduces or eliminates the skin sampling variations caused by operators, while the stiffer polyester backing creates a more uniformed skin sample collection across the patch stripping (i.e., reduces wrinkling). In some instances, adhesive components are homogenous. In some instances, adhesives comprise a first layer comprising a first component and a second layer comprising a second component. In some instances, adhesives comprise a first layer comprising a first component and a second layer comprising a second component, wherein the first layer comprises a rubber adhesive and the second layer comprises an acrylic adhesive.

Adhesives may comprise compositions as described in U.S. Pat. No. 5,625,005, incorporated herein by reference in its entirety. In some instances, adhesives comprise graft copolymer acrylates. In some instances, adhesives are generated by reacting at least one alkyl acrylate ester containing from about 4 to about 8 carbon atoms in the alkyl group in the presence of a macromer selected from the group consisting of ethylene-butylene and ethylene-propylene macromers and mixtures thereof, each of said macromers having a molecular weight of from about 2,000 to about 30,000. In some instances, adhesives comprise on a percent-by-weight basis, from about 35 to about 100 percent by weight of the total acrylate backbone of one or more alkyl acrylate esters (or vinyl esters) containing about 4 to about 8 carbon atoms in the alkyl group. In some instances, alkyl acrylate esters include n-butyl acrylate, 2-ethyl hexyl acrylate, and isooctyl acrylate. In some instances, vinyl esters include vinyl acetate, vinyl butyrate, vinyl propionate, vinyl isobutyrate, vinyl valerate, and vinyl versitate.

Adhesives may comprise compositions as described in U.S. Pat. No. 6,642,298, which is incorporated herein by reference in its entirety. In some instances, adhesives comprise an acrylic polymer copolymerized with a rubber macromer. In some instances, the polymer comprises at least one alkyl acrylate monomer containing from about 4 to about 18 carbon atoms in the alkyl group and at least one monomer whose homopolymer has a glass transition temperature greater than about 0° C., and wherein the macromer has a glass transition temperature of about −30° C. or less. In some instances, an adhesive comprises an acrylic polymer copolymerized with a rubber macromer (macromer), the polymer comprising at least one alkyl acrylate monomer containing from about 4 to about 18 carbon atoms in the alkyl group, and wherein the polymer is crosslinked using a titanium crosslinking agent. In some instances, the macromer comprises poly(ethylene-butylene), poly(ethylene-propylene) or poly(ethylene-butylene-propylene). In some instances, the macromer has a molecular weight of from about 2,000 to about 10,000. In some instances, the adhesive comprises methyl acrylate and hydroxyethyl acrylate or hydroxypropyl methacrylate. In some instances, at least one alkyl acrylate monomer is 2-ethylhexyl acrylate, said at least one monomer is methyl acrylate and said at least one hydroxy functional monomer is hydroxyethyl acrylate.

Adhesives may comprise compositions as described in U.S. Pat. No. 7,396,871, which is incorporated herein by reference in its entirety. In some instances, the adhesive comprises a rubber modified acrylic and/or vinyl resin comprising the mini-emulsion polymerization product of at least one rubber compound substantially dissolved in at least one acrylic and/or vinyl monomer, wherein said resin comprises a rubber portion derived from said rubber compound and an acrylic and/or vinyl portion derived from said acrylic and/or vinyl monomer. In some instances, the at least one rubber compound is selected from one or more of the group consisting of natural rubber, butyl rubber, isoprene rubber, chloroprene rubber, neoprene rubber, polybutadiene rubber, nitrile-butadiene rubber, styrene-butadiene rubber, polypentanamer, and ethylene-propylene-diene terpolymer. In some instances, the acrylic monomer and/or vinyl monomer is selected from the group consisting of styrene, α-methyl styrene, vinyl naphthalene, vinyl toluene, chloromethyl styrene, methyl acrylate, acrylic acid, methacrylic acid, methyl methacrylate, ethyl acrylate, ethyl methacrylate, butyl acrylate, butyl methacrylate, isobutyl acrylate, isobutyl methacrylate, ethylhexyl acrylate, ethylhexyl methacrylate, lauryl methacrylate, lauryl acrylate, octyl acrylate, octyl methacrylate, glycidyl methacrylate, allyl methacrylate, vinyl methacrylate, acetoacetoxyethyl acrylate, acetoacetoxyethyl methacrylate, acetoacetoxypropyl acrylate, acetoacetoxypropyl methacrylate, hydroxybutenyl methacrylate, the allyl ester of maleic acid, the diallyl ester of maleic acid, poly(allyl glycidyl ether), alkyl crotonates, vinyl cetate, di-n-butyl maleate, di-octylmaleate, acrylonitrile, diacetone acrylamide, acrylamide, methacrylamide, hydroxyethyl methacrylate, hydroxyethyl acrylate, acrylonitrile, t-butylaminoethyl methacrylate, dimethylaminoethyl methacrylate, diethylaminoethyl methacrylate, N, N-dimethylaminopropyl methacrylamide, 2-t-butylaminoethyl methacrylate, N, N-dimethylaminoethyl acrylate, N-(2-methacryloyloxy-ethyl)ethylene urea, and methacrylamidoethylethylene urea.

Adhesives may comprise compositions as described in US Publication No. 2008/0251201, which is incorporated herein by reference in its entirety. In some instances, adhesives comprise general compositions of poly(mneth)acrylate; polyvinyl ether; diene rubber such as natural rubber, polyisoprene, and polybutadiene; polyisobutylene; polychloroprene; butyl rubber; butadiene-acrylonitrile polymer; thermoplastic elastomer; block copolymers such as styrene-isoprene and styrene-isoprene-styrene (SIS) block copolymers, ethylene-propylene-diene polymers, and styrene-butadiene polymers; poly-alpha-olefin; amorphous polyolefin; silicone; ethylene-containing copolymer such as ethylene vinyl acetate, ethylacrylate, and ethyl methacrylate; polyurethane; polyamide; epoxy; polyvinylpyrrolidone and vinylpyrrolidone copolymers; polyesters; and mixtures or blends of the above. Adhesives in some instances comprise additives including, but not limited to, tackifiers, plasticizers, fillers, antioxidants, stabilizers, pigments, diffusing materials, curatives, fibers, filaments, and solvents.

Adhesives are in some instances an acrylic based adhesive, but other adhesives are contemplated as well and may be used. Such other adhesives include those based on silicones or based on polyolefins as disclosed in Handbook of Pressure Sensitive Adhesive Technology (third edition) D. Satas, Ed. Satas and Associates, Warwick R.I./USA, 1989 on pages 550-556 and 423-442 respectively.

Adhesives may comprise compositions as described in WIPO Publication No. WO 2014/130507, which is incorporated herein by reference in its entirety. In some instances, patches described herein comprise one or more adhesive layers. In some instances, patches comprise a first adhesive layer and a second adhesive layer. In some instances, the first adhesive layer comprises an acrylic based adhesive, a rubber based adhesive, or a combination of two or more thereof. In some instances, the first layer comprises polyisoprene, polybutadiene, styrenebutadiene polymers, styrene-butadiene block copolymers, multi-armed repeating styrene-butadiene copolymers, styrene-isoprene-styrene polymers, styrene-butadienestyrene polymers, styrene-isoprene polymers, styreneisoprene block copolymers, and multi-armed repeating styrene-isoprene copolymers, or a combination of two or more thereof. In some instances, the second layer comprises an adhesive comprising a monomer chosen from methyl acrylate, ethyl acrylate, n-propyl acrylate, isopropyl acrylate, n-butyl acrylate, isobutyl acrylate, n-amyl acrylate, isoamyl acrylate, n-hexyl acrylate, isohexyl acrylate, cyclohexyl acrylate, isooctyl acrylate, 2-ethyl hexyl acrylate, decyl acrylate, lauryl acrylate, stearyl acrylate, isobornyl acrylate, methyl methacrylate, ethyl methacrylate, n-propyl methacrylate, isopropyl methacrylate, n-butyl methacrylate, isobutyl methacrylate, n-amyl methacrylate, isoamyl methacrylate, n-hexyl methacrylate, isohexyl methacrylate, cyclohexyl methacrylate, isooctyl methacrylate, 2-ethyl hexyl methacrylate, decyl methacrylate, lauryl methacrylate, stearyl methacrylate, and isobornyl methacrylate, or a combination of two or more thereof.

Adhesive patches may be clear, transparent or opaque depending on the application. In some instances, the patch is opaque. In some instances, the patch is clear. In some instances, the patch is transparent. In some instances, the patch has an opacity of about 1%, 2%, 5%, 8%, 10%, 15%, 20%, 25%, 30%, 40%, 50%, 60%, 70%, 80%, 90%, 95%, or about 98%. In some instances, the patch has an opacity of at least 1%, 2%, 5%, 8%, 10%, 15%, 20%, 25%, 30%, 40%, 50%, 60%, 70%, 80%, 90%, 95%, or at least 98%. In some instances, the patch has an opacity of no more than 1%, 2%, 5%, 8%, 10%, 15%, 20%, 25%, 30%, 40%, 50%, 60%, 70%, 80%, 90%, 95%, or no more than 98%. In some instances, the patch has an opacity after removing skin cells one or more times (peeling). In some instances, the patch has an opacity of about 1%, 2%, 5%, 8%, 10%, 15%, 20%, 25%, 30%, 40%, 50%, 60%, 70%, 80%, 90%, 95%, or about 98% after 1 peeling of skin cells. In some instances, the patch has an opacity of at least 1%, 2%, 5%, 8%, 10%, 15%, 20%, 25%, 30%, 40%, 50%, 60%, 70%, 80%, 90%, 95%, or at least 98% after 1 peeling of skin cells. In some instances, the patch has an opacity of no more than 1%, 2%, 5%, 8%, 10%, 15%, 20%, 25%, 30%, 40%, 50%, 60%, 70%, 80%, 90%, 95%, or no more than 98% after 1 peeling of skin cells. In some instances, an adhesive patch comprises a haze value of less than about 50%, 45%, 40%, 30%, 25%, 20%, 15%, 10%, or less than about 5% as measured by ASTM D1003. In some instances, patches are distinguishable from one another by color, pattern, or other marking. In some instances, patches comprise one or more colors such as red, green, orange, pink, blue, grey, black, brown, cyan, purple, and yellow. In some instances, patches comprise one or more patterns, optionally with color. In some instances, color indicates one or more properties of the patch. In some instances, color indicates which area of the body has been or will be sampled by the patch. (e.g., pink=forehead, blue=chin, etc.). In some instances, at least two adhesive patches comprise the same color. In some instances, at least two adhesive patches comprise different colors.

Adhesive patches may comprise a matrix material. The matrix material in some instances is sufficiently sticky to adhere to a skin sample. The matrix material is not so sticky that is causes scarring or bleeding or is difficult or painful to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant. In some instances, a single patch is applied a single time to a single area or region. In some instances, a single patch is applied multiple times to a single area or region. In some instances, a single patch is applied a single time to multiple areas or regions. In some instances, multiple patches are applied at a single time to a single area or region. In some instances, multiple patches are applied multiple times to a single area or region. In some instances, multiple patches are applied multiple times to multiple areas or regions. In some instances, greater than 2 applications in the same area or region results in no more than 80, 70, 60, 50, 40, 35, 30, 25, 20, 17, 15, 12, 10, or no more than 5 g/m²/h) transepidermal water loss (TEWL). In some instances, greater than 4 applications in the same area or region results in no more than 80, 70, 60, 50, 40, 35, 30, 25, 20, 17, 15, 12, 10, or no more than 5 g/m²/h) transepidermal water loss (TEWL). In some instances, greater than 8 applications in the same area or region results in no more than 80, 70, 60, 50, 40, 35, 30, 25, 20, 17, 15, 12, 10, or no more than 5 g/m²/h) transepidermal water loss (TEWL). In some instances, greater than 6 applications in the same area or region results in no more than 80, 70, 60, 50, 40, 35, 30, 25, 20, 17, 15, 12, 10, or no more than 5 g/m²/h) transepidermal water loss (TEWL).

Adhesive patches may comprise a flexible material, enabling the patch to conform to the shape of the skin surface upon application (or backing layer). In some instances, patches comprise an adhesive matrix present in the first collection area. In some instances, at least the first collection area is flexible. In some instances, the tab is plastic. In an illustrative example, the adhesive patch does not contain latex, silicone, or both. In some embodiments, the adhesive patch is made of a clear or transparent material, so that the skin sampling area of the subject is visible after application of the adhesive patch to the skin surface. The transparency, e.g., providing visibility through the patch, ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical. In some embodiments the length of the patch (including both adhesive and non-adhesive handling areas) is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical. Without being bound by theory the length of a patch applied to the skin is comparable to the wrinkling wavelength to avoid the wavy structure on static patch before peel. In some instances, the longest linear dimension of the patch no more than 15, 12, 10, 8, 6, 5, 4, 3, 2, or no more than 1 cm. In some instances, the longest linear dimension of the first collection area is no more than 15, 12, 10, 8, 6, 5, 4, 3, 2, or no more than 1 cm.

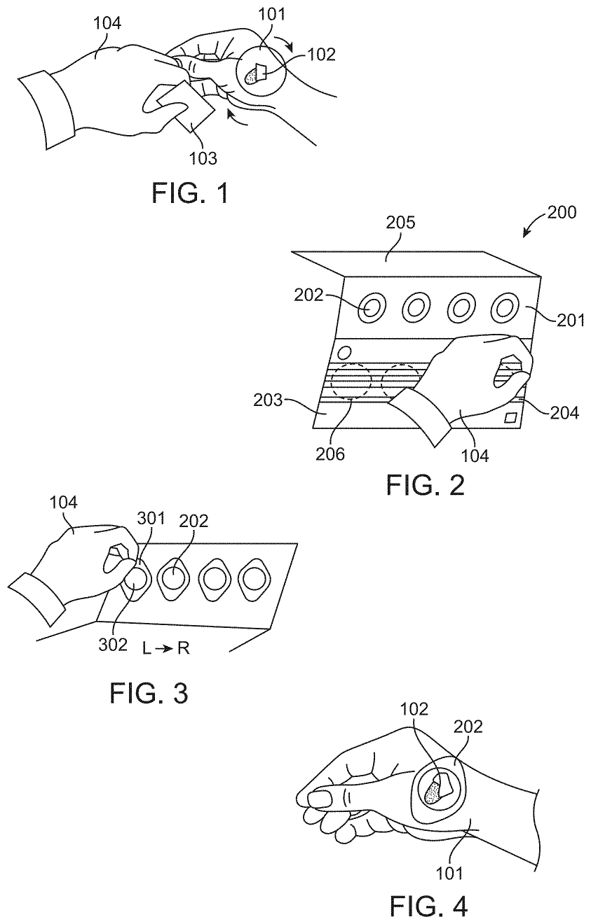

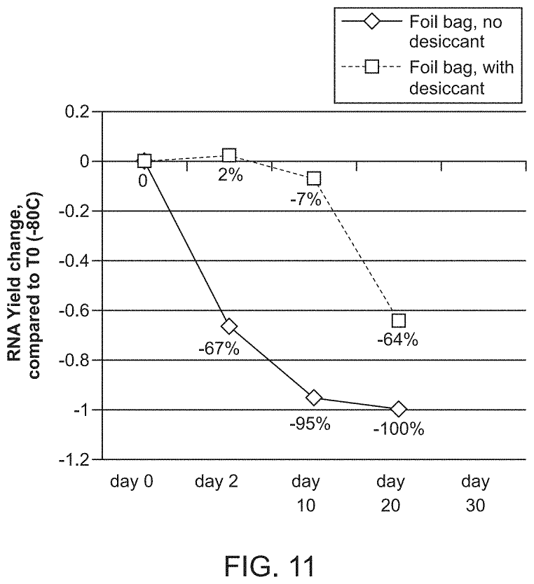

Patches may be configured for any size, color or shape. In some instances, patches are configured to adhere to specific areas of the body (e.g., face, head, or other area). In some instances, patches are configured as a single sheet covering the entire face. In some instances, multiple patches are configured to sample skin different parts of the body. In some instances multiple patches are configured to sample skin from the face or different parts of the face. In some instances, patches are used as disclosed in FIGS. 11-13 of US Publication No. 2016/0279401, which is incorporated by reference in its entirety; or FIGS. 1-4 of US Publication No. 20030167556, which is incorporated by reference in its entirety.

In some embodiments, a skin collection device such as an adhesive patch comprises a shape. The skin collection device may include one shape or may include multiple shapes. A kit may include skin collection devices having separate shapes, for example 1, 2, 3, 4, 5, 6, 7, 8, 9, 10, or more different shaped collection devices. Examples of shapes include circles, ovals, squares, and the like. A shape may be straight. A shape may be generally composed of straight line segments. For example, the shape may include an angle (e.g. acute angle, obtuse angle, or right angle), balbis, concave polygon, constructible polygon, convex polygon, cyclic polygon, equiangular polygon, equilateral polygon, penrose tile, polyform, regular polygon, simple polygon, or tangential polygon. The shape may include a polygon with a specific number of sides, such as a triangle—3 sides, acute triangle, equilateral triangle, heptagonal triangle, isosceles triangle, golden triangle, obtuse triangle, rational triangle, right triangle, 30-60-90 triangle, isosceles right triangle, kepler triangle, scalene triangle, quadrilateral—4 sides, cyclic quadrilateral, kite, parallelogram, rhombus (equilateral parallelogram), lozenge, rhomboid, rectangle, square (regular quadrilateral), tangential quadrilateral, trapezoid, isosceles trapezoid, pentagon—5 sides, hexagon—6 sides, lemoine hexagon, heptagon—7 sides, octagon—8 sides, nonagon—9 sides, decagon—10 sides, hendecagon—11 sides, dodecagon—12 sides, tridecagon—13 sides, tetradecagon—14 sides, pentadecagon—15 sides, hexadecagon—16 sides, heptadecagon—17 sides, octadecagon—18 sides, enneadecagon—19 sides, icosagon—20 sides, star polygon—there are multiple types of stars, pentagram—star polygon with 5 sides, hexagram—star polygon with 6 sides, star of David, heptagram—star polygon with 7 sides, octagram—star polygon with 8 sides, star of Lakshmi, enneagram—star polygon with 9 sides, decagram—star polygon with 10 sides, hendecagram—star polygon with 11 sides, dodecagram—star polygon with 12 sides, or apeirogon—generalized polygon with countably infinite set of sides. The shape may be curved. The shape may be composed of circular arcs. For example, the shape may include an annulus, arbelos, circle, archimedes' twin circles, bankoff circle, circular triangle, reuleaux triangle, circumcircle, disc, incircle and excircles of a triangle, nine-point circle, circular sector, circular segment, crescent, lens, vesica piscis (fish bladder), lune, quatrefoil, reuleaux polygon, reuleaux triangle, salinon, semicircle, tomahawk, trefoil, triquetra, or heart shape. In some embodiments, the shape may not be composed of circular arcs. For example, the shape may include an Archimedean spiral, astroid, cardioid, deltoid, ellipse, heartagon, lemniscate, oval, cartesian oval, cassini oval, oval of booth, ovoid—similar to an oval, superellipse, taijitu, tomoe, or magatama shape.

The shape may be based on a skin collection area. For example, the skin collection device may include a single large patch, include a face mask, be shaped for a forehead (e.g., be kidney shaped), be shaped to go under eyes (e.g. crescent), be shaped to cover at least part of a nose (e.g., butterfly shaped), be shaped to cover at least part of a right cheek, be shaped to cover at least part of a left cheek, may be postauricular, may be shaped to cover at least part of a right or left hand, or may be shaped to cover at least part of a right or left foot.

Parameters which effect the static wrinkling may include elasticity of the skin, elasticity of the backing layer, strength of the adhesive, and geometric parameters such as the length and width of the tape. One or more of these parameters may affect a wavelength and frequency of wrinkling patterns in the backing layer. Of these, the elasticity of the skin may not be readily controllable. For example, it may be a property of the skin to which the patch may adhere. An adhesive patch may comprise one or more of the following properties: a backing thickness greater than 3 mil, a longest dimension less than 10 cm, and a backing layer with an elastic modulus between 200 and 2000 PSI. An adhesive patch may comprise one or more of the following properties: a backing thickness greater than 3 mil, a longest dimension less than 5 cm, and a backing layer with an elastic modulus between 500 and 1500 PSI. An adhesive patch may comprise one or more of the following properties: a backing thickness greater than 3 mil, a longest dimension less than 5 cm, and a backing layer with an elastic modulus between 1000 and 2000 PSI. An adhesive patch may comprise an elastic modulus of from about 1000 to about 2000 Psi, about 500 to about 3000 Psi, about 250 to about 2000 Psi, about 400 to about 2000 Psi, about 500 to about 1500 Psi, about 750 to about 2000 Psi, about 1000 to about 3000 Psi, or about 500 to about 2500 Psi; a backing thickness greater than 3 mil; and a longest dimension less than 10 cm. An adhesive patch may comprise a longest dimension of about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm; a backing thickness greater than 3 mil; and a longest dimension less than 10 cm. An adhesive patch may comprise a backing thickness of about 3 mil, about 4 mil, about 5 mil, about 6 mil, about 7 mil, about 8 mil, about 9 mil, about 10 mil, about 20 mil, about 30 mil, about 40 mil, about 50 mil, about 60 mil, about 70 mil, about 80 mil, about 90 mil, about 100 mil, or about 125 mil; a longest dimension less than 10 cm; and a backing layer with an elastic modulus between 200 and 2000 PSI.

In further embodiments, the adhesive patch is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector. The peelable release sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The peelable release sheet is configured to hold about 12 adhesive patches. The peelable release sheet is configured to hold about 11 adhesive patches. The peelable release sheet is configured to hold about 10 adhesive patches. The peelable release sheet is configured to hold about 9 adhesive patches. The peelable release sheet is configured to hold about 8 adhesive patches. The peelable release sheet is configured to hold about 7 adhesive patches. The peelable release sheet is configured to hold about 6 adhesive patches. The peelable release sheet is configured to hold about 5 adhesive patches. The peelable release sheet is configured to hold about 4 adhesive patches. The peelable release sheet is configured to hold about 3 adhesive patches. The peelable release sheet is configured to hold about 2 adhesive patches. The peelable release sheet is configured to hold about 1 adhesive patch.

The adhesive patch is applied to the skin and removed from the skin. After removing the used adhesive patch from the skin surface, the patch stripping method further comprises storing the used patch on a placement area sheet, where the patch remains until the skin sample is isolated or otherwise utilized. The used patch is configured to be stored on the placement area sheet for at least 1 week at temperatures between −80° C. and 30° C. In some embodiments, the used patch is configured to be stored on the placement area sheet for at least 5 days, at least 10 day, at least 2 weeks, at least 3 weeks, at least 1 month, at least 2 months, at least 3 months, at least 4 months, at least 5 months, and at least 6 months at temperatures between −80° C. to 30° C. In some instances, patches are stored with a desiccant.

Skin collector kits may comprise an adhesive matrix. In some instances, the adhesive matrix comprises at least 3, 5, 8, 10, 11, 12, 13, 14, 15, 18, 20, or at least 25 oz/in² loop tackiness. In some instances, the adhesive matrix comprises 3-25, 3-20, 3-15, 5-20, 8-20, 10-15, 15-24, 10-20, or 1-20 oz/in² loop tackiness. In some instances, the adhesive matrix comprises a working temperature range from −40 to 176° F., −40 to 150° F., −40 to 130° F., −30 to 176° F., −20 to 176° F., −10 to 176° F., or −40 to 200° F. In some instances, the backing layer comprises at least 5, 8, 10, 12, 15, 18, 20, 23, 25, 30, or at least 55 lb/inch tensile force. In some instances, the backing layer comprises about 5, 8, 10, 12, 15, 18, 20, 23, 25, 30, or about 55 lb/inch tensile force. In some instances, the backing layer comprises 5-55, 5-40, 5-30, 5-25, 1-50, 10-20, 10-30, 15-30, 15-45, 20-45, 25-40, 30-50, or 25-60 lb/inch tensile force. In some instances, the backing layer comprises about 50, 80, 100, 120, 150, 180, 200, 230, 250, 300, 400, or about 500 mN tear strength. In some instances, the backing layer comprises 50-550, 50-400, 50-300, 50-250, 100-500, 100-200, 100-300, 150-300, 150-450, 200-450, 250-400, 300-500, or 250-600 mN tear strength.

In some instances, one or more components of the skin collector kit may be water soluble. In some instances, the adhesive patch is water soluble. In some instances, one or more of the backing layer and adhesive matrix are water soluble. In some instances, the placement area sheet is water soluble. In some instances, backing layer or adhesive matrix is configured to dissolve during skin sample lysis. In some instances, the adhesive patch is dissolvable in aqueous solution in no more than 10, 15, 20, 30, 40, 50, 60, 90, or not more than 120 seconds. In some instances, the adhesive patch is dissolvable in an aqueous solution in no more than 10, 15, 20, 30, 40, 50, 60, 90, or not more than 120 seconds in an aqueous solution. In some instances, the adhesive patch is dissolvable in no more than 10, 15, 20, 30, 40, 50, 60, 90, or not more than 120 seconds in an aqueous solution having a temperature of no more than 30 degrees C. In some instances, the adhesive patch is dissolvable in no more than 10, 15, 20, 30, 40, 50, 60, 90, or not more than 120 seconds in an aqueous solution having a temperature of no more than 20 degrees C. In some instances, wherein the adhesive patch has shelf life of at least 1, 2, 3, 6, 8, 12, 14, 16, or at least 24 months. In some instances, wherein the adhesive patch has a shelf life of at least 1, 2, 3, 6, 8, 12, 14, 16, or at least 24 months at a temperature between −80 degrees and 30 degrees C. In some instances, wherein the adhesive patch has a shelf life of at least 1, 2, 3, 6, 8, 12, 14, 16, or at least 24 months at a temperature no greater than 30 degrees C. In some instances, wherein the adhesive patch has a shelf life of at least 1, 2, 3, 6, 8, 12, 14, 16, or at least 24 months at a temperature no greater than 25 degrees C. In some instances, wherein the adhesive patch has a shelf life of at least 1, 2, 3, 6, 8, 12, 14, 16, or at least 24 months at a temperature no greater than 20 degrees C. In some instances, wherein the adhesive patch has a shelf life of at least 1, 2, 3, 6, 8, 12, 14, 16, or at least 24 months at a temperature no greater than 10 degrees C. In some instances, wherein the adhesive patch has a shelf life of at least 1, 2, 3, 6, 8, 12, 14, 16, or at least 24 months at a temperature no greater than 5 degrees C. In some instances, wherein the adhesive patch has a shelf life of at least 1, 2, 3, 6, 8, 12, 14, 16, or at least 24 months at a temperature no greater than 0 degrees C. In some instances, wherein the adhesive patch has a shelf life of at least 1, 2, 3, 6, 8, 12, 14, 16, or at least 24 months at a temperature no greater than −20 degrees C. In some instances, wherein the adhesive patch has a shelf life of at least 1, 2, 3, 6, 8, 12, 14, 16, or at least 24 months at a temperature no greater than −40 degrees C. In some instances, wherein the adhesive patch has a shelf life of at least 1, 2, 3, 6, 8, 12, 14, 16, or at least 24 months at a temperature of no more than 30 degrees C.

An adhesive patch may comprise a water soluble adhesive. Current skin sample collection tools in some instances comprise non-invasive sample collection and target analyte tests using an adhesive patch that comprises two parts: (a) a layer of non-water soluble adhesive (styrene-butadiene diblock copolymer) on (b) a thin backing sheet of thermoplastic polyurethane (TPU) film. This non-water soluble adhesive in some instances may cause the sample-loaded patches to stick together (self-fold or between patches) during sample lysis incubation, which prevents target analytes, including proteins and nucleic acids (DNA and RNA) from releasing from the samples collected on the patch to the lysis solution (reduces target analyte recovery yields) and the use of a larger sample collection patch for sample collection (due to a higher incident of patch self-fold sticking in a sample lysis incubation tube), in some instances limiting this non-invasive sample application tool to analyte tests, such as genomic tests, applications that may run on minute quantity of samples, and the incubation of multiple sample-loaded patches in one tube (due to sticking between patches, so each patch has to be incubated in a separate tube) may increase the cost on sample preparation for some analyte tests. The non-water soluble TPU backing sheet of the patch in some instances has disadvantages, e.g., the TPU film is removed from the lysis tube at the end of lysis incubation (before magnetic beads are added to the lysis tube), to prevent magnetic beads from sticking to the adhesive on the TPU film (those beads will get lost from the process, together with the sample nucleic acids bound on these beads). The process of removing TPU film in some instances presents challenges such as interrupting the workflow of the extraction process (increase in both labor work, time), a fully manual process that prevents process automation, increasing the chance of cross-contamination between samples, and potentially causing loss of protein- or nucleic acid-containing sample lysis solution to be ruined or made unusable due to the use of TPU films), reducing the sample target analyte (e.g., protein or nucleic acid) yields. In some instances, use of water soluble adhesives and/or patches improves performance of the non-invasive sampling systems and methods described herein. Soluble adhesive patches are used to non-invasively collect skin samples for analyte (e.g., genomic) testing, i.e., collecting skin samples with (water) soluble adhesive patches and these sample-loaded adhesive patches (including adhesive and backing sheet) can more easily dissolve in the lysis solution with the collected skin samples during sample extraction. These soluble adhesive patches in some instances allow all sample-loaded patches (especially when multiple patches are used for collection) to incubate in one lysis tube (reducing sample prep cost and time) and eliminating a manual step of removing the backing films from lysis tubes. In some instances, use of soluble adhesive patches allow for automation of the sample process to save time and labor costs, and reduces the chance of cross-contamination and lost samples. In some instances, use of soluble adhesive patches provides increased utilization (up to 100%) of all collected skin tissues for an analyte (e.g., nucleic acid) extraction. In some instances, use of soluble adhesive patches provides at least 50%, 60%, 70%, 80%, 90%, 95%, or at least 99% utilization of all collected skin tissues for an analyte (e.g., nucleic acid) extraction. In some instances, all skin tissues on soluble patches are released to the lysis solution, compared to the non-soluble patches where some skin tissues may still remain trapped in the non-soluble adhesive layers after lysis incubation.

Soluble adhesive patches may provide increased utilization of human or microbial proteins (and/or polypeptides). In some instances, use of soluble adhesive patches provides increased utilization (up to 100%) of all collected skin tissues for human or microbial protein extraction. In some instances, use of soluble adhesive patches provides at least 50%, 60%, 70%, 80%, 90%, 95%, or at least 99% utilization of all collected skin tissues for human or microbial protein extraction. In some instances, all skin tissues on soluble patches are released to the lysis solution, compared to the non-soluble patches where some skin tissues may still remain trapped in the non-soluble adhesive layers after lysis incubation.

Soluble adhesive patches may provide increased utilization of human or microbial nucleic acids. In some instances, nucleic acids comprise one or more of DNA, RNA, genomic DNA, or cDNA. In some instances, use of soluble adhesive patches provides increased utilization (up to 100%) of all collected skin tissues for human or DNA extraction. In some instances, use of soluble adhesive patches provides at least 50%, 60%, 70%, 80%, 90%, 95%, or at least 99% utilization of all collected skin tissues for human or microbial DNA extraction. In some instances, all skin tissues on soluble patches are released to the lysis solution, compared to the non-soluble patches where some skin tissues may still remain trapped in the non-soluble adhesive layers after lysis incubation. In some instances, use of soluble adhesive patches provides increased utilization (up to 100%) of all collected skin tissues for human or RNA extraction. In some instances, use of soluble adhesive patches provides at least 50%, 60%, 70%, 80%, 90%, 95%, or at least 99% utilization of all collected skin tissues for human or microbial RNA extraction.

Water soluble adhesives may generally include adhesives formed by copolymerization of a hydrophilic monomer with a monomer that is used in an adhesive resin. Monomers used in adhesive resins may include monomers of one or more adhesive matrix materials described herein, for example, one or more of acrylics, silicones, and hydrocarbon rubbers (like butyl rubber, styrene-butadiene rubber, ethyl-vinyl acetate polymers, styrene-isoprene-butadiene rubbers), or combination thereof. Monomers used in adhesive resins may include monomers of one or more adhesive matrix materials such as, for example, polyvinylpyrrolidone, polyacrylamide, polyacrylic acid, polyvinyl ethers, cellulose ethers, natural or synthetic gums, and polyethers (e.g., polyethylene glycol). Formulations of adhesive resins may include various types of water soluble and/or water dispersible salts, plasticizers, tackifiers, and surfactants. Tackifiers and plasticizers may be used to improve adhesion in formulations of adhesive resins. Example tackifiers and plasticizers may include one or more of, for example, ethoxylates, glucosides, rosins, and polyols.

Water soluble adhesives may generally include adhesives formed by conversion of an acrylic adhesive, which may not be sufficiently water soluble, to a more water soluble adhesive. Water solubility may be increased, for example, by neutralization of a carboxylic group in a pendant group of the monomer. The resultant polymer may, optionally, be plasticized with polyethylene glycol or polypropylene glycol. In an example, adhesive monomers such as, for example, butyl acrylate, acrylic acid, di-2-ethylhexyl fumarate, and/or vinyl acetate may be copolymerized, followed by the addition of an ethoxylated tert-N-alkyl diamine (an ethoxylated surfactant) as a plasticizer and/or tackifier and potassium hydroxide (neutralization agent). See for example, U.S. Pat. No. 3,441,430, which is incorporated herein by reference in its entirety.

Water soluble adhesives may generally include adhesives formed from acrylic acid and acrylamide, a polyhydric alcohol surfactant (tackifier/plasticizer), and a caustic (neutralization agent). See for example, U.S. Pat. No. 4,388,432, which is incorporated herein by reference in its entirety.

Water soluble adhesives may generally include adhesives formed from copolymers of acrylic acid and acrylates. These copolymers can be neutralized with aminopropanol followed by the addition of glycol ether. See, for example, JP Patent No. JP-56-7007, which is incorporated herein by reference in its entirety.

Water soluble adhesives may generally include adhesives formed from copolymers of 2-ethylhexyl acrylate, hydroxyethyl methacrylate, and acrylic acid. The copolymer may be neutralized with sodium hydroxide in methanol to make a water soluble adhesive. The formulation may include polyethylene glycol (tackifier/plasticizer) and polypropylene glycol diglycidyl ether (tackifier/plasticizer). See, for example, JP Patent No. JP-57-156456, which is incorporated herein by reference in its entirety.

Water soluble adhesives may generally include adhesives formed from polyethylene glycol, polypropylene glycol, or similar hydrophilic polymers or surfactants with hydroxyl or amine groups grafted to acrylic acid pendant groups on the adhesive polymers.

Water soluble adhesives may generally include adhesives formed from polyvinyl alcohol, cellulose ethers, and blends of such polymers. The adhesive formulation may be blended with water, dispersible/soluble additives, and/or other thermoplastics.

In some instances, the placement area sheet comprises a removable liner, provided that prior to storing the used patch on the placement area sheet, the removable liner is removed. The placement area sheet is configured to hold a plurality of adhesive patches, including, but not limited to, 16, 14, 12, 11, 10, 9, 8, 7, 6, 5, 4, 3, 2, 1 patches, from about 2 to about 8 patches, from about 2 to about 7 patches, from about 2 to about 6 patches, from about 2 to about 4 patches, from about 3 to about 6 patches, from about 3 to about 8 patches, from about 4 to about 10 patches, from about 4 to about 8 patches, from about 4 to about 6 patches, from about 4 to about 5 patches, from about 6 to about 10 patches, from about 6 to about 8 patches, or from about 4 to about 8 patches. In some embodiments, the placement area sheet is configured to hold about 12 adhesive patches. In some embodiments, the placement area sheet is configured to hold about 11 adhesive patches. In some embodiments, the placement area sheet is configured to hold about 10 adhesive patches. In some embodiments, the placement area sheet is configured to hold about 9 adhesive patches. In some embodiments, the placement area sheet is configured to hold about 8 adhesive patches. In some embodiments, the placement area sheet is configured to hold about 7 adhesive patches. In some embodiments, the placement area sheet is configured to hold about 6 adhesive patches. In some embodiments, the placement area sheet is configured to hold about 5 adhesive patches. In some embodiments, the placement area sheet is configured to hold about 4 adhesive patches. In some embodiments, the placement area sheet is configured to hold about 3 adhesive patches. In some embodiments, the placement area sheet is configured to hold about 2 adhesive patches. In some embodiments, the placement area sheet is configured to hold about 1 adhesive patch.

In some embodiments, the used patch is stored so that the matrix containing, skin facing surface of the used patch is in contact with the placement area sheet. In some instances, the placement area sheet is a panel of the tri-fold skin sample collector. In some instances, the tri-fold skin sample collector may further comprise a clear panel. The tri-fold skin sample collector may be labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive patches stored on a peelable release panel. In some instances, the tri-fold skin sample collector further comprises a placement area panel with a removable liner. In some embodiments, the patch stripping method involves removing an adhesive patch from the tri-fold skin sample collector peelable release panel, applying the adhesive patch to a skin sample, removing the used adhesive patch containing a skin sample and placing the used patch on the placement area sheet. In some instances the placement area panel is a single placement area panel sheet. In some embodiments, the identity of the skin sample collected is indexed to the tri-fold skin sample collector or placement area panel sheet by using a barcode or printing patient information on the collector or panel sheet. In some embodiments, the indexed tri-fold skin sample collector or placement sheet is sent to a diagnostic lab for processing. In some embodiments, the used patch is configured to be stored on the placement panel for at least 1 week at temperatures between −80° C. and 30° C., e.g., −70, −60, −25, 0, 5, 10, 25, and 30 degrees. In some embodiments, the used patch is configured to be stored on the placement panel for at least 1 week at about room temperature (−25° C.). In some embodiments, the used patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 30° C. In some embodiments the indexed tri-fold skin sample collector or placement sheet is sent to a diagnostic lab using UPS or FedEx.

In some embodiments, the patch stripping method further comprises preparing the skin sample prior to application of the adhesive patch. Preparation of the skin sample can include, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from a surface including, but not limited to, the face, head, neck, arm, chest, abdomen, back, leg, hand, and/or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms. In some instances, the skin surface comprises a lesion (i.e., lesional). In some instances, the skin surface does not comprise a visible lesion (i.e., non-lesional). In some instances, non-lesional skin is obtained from a subject having a disease or condition but without visible lesions on the skin. In some instances, the skin surface comprises normal skin.

The patch stripping method, devices, and systems described herein are optionally useful for the collection of a skin sample from a non-lesional skin surface. The patch stripping method, devices, and systems described herein are optionally useful for the collection of a skin sample from a normal or healthy skin surface. The patch stripping method, devices, and systems described herein are optionally useful for the collection of a skin sample from a skin lesion or lesional skin surface. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion.

The adhesive skin sample collection kit comprises at least one adhesive patch, a sample collector, and an instructions for use. The instructions for use may be provided in the form of a sheet of paper or papers (e.g, booklet or brochure). The instructions for use may be provided in the form of a link or QR code for the user to access electronically or digitally. In an exemplary embodiment, the sample collector is a tri-fold skin sample collector comprising a peelable release panel comprising at least one adhesive patch, a placement area panel comprising a removable liner, and a clear panel. The tri-fold skin sample collector may further comprise a barcode and/or an area for transcribing patient information. The adhesive skin sample collection kit is configured to include a plurality of adhesive patches, including but not limited to 16, 14, 12, 11, 10, 9, 8, 7, 6, 5, 4, 3, 2, 1 patches, from about 2 to about 8 patches, from about 2 to about 7 patches, from about 2 to about 6 patches, from about 2 to about 4 patches, from about 3 to about 6 patches, from about 3 to about 8 patches, from about 4 to about 10 patches, from about 4 to about 8 patches, from about 4 to about 6 patches, from about 4 to about 5 patches, from about 6 to about 10 patches, from about 6 to about 8 patches, or from about 4 to about 8 patches. In some embodiments, the instructions for use provides the kit operator all of the necessary information for carrying out the patch stripping method. The instructions for use sheet preferably includes diagrams to illustrate the patch placement and/or stripping method.

In some instances, the adhesive skin sample collection kit provides all the necessary components for performing the patch stripping method. In some instances, a kit comprises one or more of at least one adhesive patch (2, 4, 6, 8, 10 or 12 adhesive patches), wherein the least one adhesive patch comprises: a backing layer comprising a collection area; a non-adhesive handling area; an adhesive matrix on a surface of the collection area, wherein the adhesive matrix is configured to adhere to an amount of a skin sample; and a packaging comprising instructions. In some embodiments, the adhesive skin sample collection kit includes a lab requisition form for providing patient information. In some embodiments, the adhesive skin sample collection kit includes a return mailing label. In some instances, the kit further comprises accessory components. Accessory components may include, but are not limited to, a marker, a resealable bag (e.g., a plastic or foil bag), gloves, and a cleansing reagent. The cleansing reagent includes, but is not limited to, an antiseptic such as isopropyl alcohol. In some instances, a skin sample collection kit may be provided in a cardboard box. In some instances, a kit comprises any of the skin collection components described herein. In some instances, a kit further comprises packaging comprising instructions. In some instances, the instructions are provided to perform one or more of the following: placing a patch on a specified area or areas of skin, marking a patch to approximately a size of a lesion on a skin; peeling a patch slowly; and/or peeling at an angle greater than about perpendicular to the skin surface. In some instances, slowly is indicated as less than about 0.5, 0.7, 0.8, 0.9, 1, 1.1, 1.2, 1.5, 2.0, or 2.5 linear inches peeled per about five seconds. In some instances, slowly is indicated as less than about 0.5, 0.7, 0.8, 0.9, 1, 1.1, 1.2, 1.5, 2.0, or 2.5 linear inches peeled per about ten seconds. In some instances, slowly is indicated as less than about 0.5, 0.7, 0.8, 0.9, 1, 1.1, 1.2, 1.5, 2.0, or 2.5 linear inches peeled per about three seconds.

A kit described herein may comprise a preservation or storage system for a collected skin sample. In some instances, a kit for non-invasive collection and analysis of a skin sample comprises at least one adhesive patch, wherein the least one adhesive patch comprises: a backing layer comprising a collection area; a non-adhesive handling area; an adhesive matrix on a surface of the collection area, wherein the adhesive matrix is configured to adhere to an amount of a skin sample; and a return or storage receptacle to receive the at least one adhesive patch. In some instances, the return or storage receptacle comprises a preservative. In some instances the storage/return receptacle comprises a pouch, bag, tube, or other receptacle. In some instances the storage/return receptacle is sealable. In some instances the storage/return receptacle comprises foil or plastic. In some instances, the preservative is a desiccant. In some instances, the preservative is configured to prevent degradation of biological molecules sampled using the collector kit. In some instances, the desiccant is configured to prevent the activity of nucleases in the skin sample. In some instances, the desiccant is configured to prevent degradation of nucleic acids in the sample. In some instances, the desiccant is configured to prevent the activity of RNases, DNases, or both RNases and DNases, and/or proteases in the skin sample and/or to prevent degradation of RNA, DNA, DNA/RNA and/or proteins in the skin sample. In some instances, the amount of the desiccant is from about 0.5 grams to about 5 grams, about 0.1 grams to about 10 grams, about 0.1 grams to about 5 grams, about 0.5 grams to about 5 grams, about 0.1, 0.5, 1, 1.5, 2.0, 2.5, 3, 3.5, 4, 4.5, 5, 5.5, 6, 8, 10, 12, 15 or about 20 grams. In some instances, the kit comprises a return pouch. In some instances, the return pouch is plastic or foil. In some instances, the return pouch is sealable. In some instances, the desiccant is silica gel.

### Tissue Sampling and Cellular Material

In some embodiments, sample collection (e.g., patch stripping) can be performed using an adhesive skin sample collection kit. The patch stripping method, in some instances, comprises applying and removing an adhesive patch to the skin surface of a subject. In some embodiments, adhesive patch comprises an adhesive matrix, wherein during application of the adhesive patch to the skin surface, an effective amount of a skin sample containing cellular material adheres to the adhesive matrix. In some embodiments, the cellular material comprises cells from the patient or subject providing the sample (e.g., human cellular material). In other embodiments, the cellular material comprises cells from a microbiome on the patient's or subject's skin (e.g., microbial cellular material). In some embodiments, the cellular material comprises cells from both the subject and the microbiome existing on the skin of the subject. In some embodiments, adhered skin sample is retained on the adhesive matrix upon removal of the patch from the skin surface. In some embodiments, adhesive patch containing the adhered skin sample is designated as a used adhesive patch. In some embodiments, adhesive patch is configured so that at least a portion of the skin sample cellular material can be harvested from a used patch.

In some embodiments, the method of collection of cellular or other material on the skin comprises using from one, two, three, four, five, six, seven, eight, nine, 10, 11, 12, 13, 14, 15, 16, 17, 18, 19, 20, or more patches in methods described herein. In some embodiments, the method of collection of cellular or other material on the skin comprises using a single patch. In some instances, patches are applied to one or more placement locations on a subject's skin. In some instance, a single patch is applied 1, 2, 3, 4, 5, 6, 8, 10, 12, or more than 12 times to a placement location. In some instance, multiple patches are applied to a placement location. In some instances, a single patch is applied to multiple placement locations. In some instances, patch color is indicative of the placement location. In some instance, 2, 4, 8, or 12 patches are applied to a placement location. In some embodiments, the method of collection of cellular or other material on the skin comprises using multiple patches. In some embodiments, the multiples patches are of the same size, color and/or shape. In some embodiments, the multiples patches are a different size, color and/or shape. In some embodiments, the adhesive skin sample collection kit for use with patch stripping methods is provided as a non-invasive means to collect skin samples with minimal discomfort. In some embodiments, the cellular material is isolated from the skin sample and can be utilized in tests that can determine the stage of disease, the risk of disease progression and a patient's likelihood of responding to a particular treatment. In some embodiments, the treatments include drug therapies and biopsy. In some embodiments, the skin sample cellular materials include proteins, nucleic acids, polypeptides, lipids, carbohydrates and small molecules. In some embodiments, target analytes include proteins, nucleic acids, polypeptides, lipids, carbohydrates and small molecules. Nucleic acids include DNA and RNA. DNA can be genomic DNA or copy DNA (cDNA).

Provided herein are methods of extracting biomolecules from skin samples. In some instances, biomolecules are extracted from patches described herein. In some instances, biomolecules comprise nucleic acids. In some instances, extraction of nucleic acids comprises one or more of lysing, binding, washing and elution of nucleic acids attached to patches. In some instances, samples are first lysed, which involves breaking the cell membrane and freeing the nucleic acid. In some embodiments, ethanol is added to the lysate to provide ideal binding conditions. In some embodiments, the binding solution is then loaded onto the RNeasy silica spin column membrane. In some embodiments, the wash buffers are added to the column and centrifuged three times to force the buffer through the column and wash away any remaining impurities from the membrane, leaving RNA bound to the silica gel. In some embodiments, the elution buffer (water) is added to the column and centrifuged to remove the nucleic acid from the membrane and the nucleic acid is collected from the bottom of the column.

Adhesive patches may be configured to minimize extractables (or leachables), which in some instances may lead to interference with proteomic or nucleic acid experiments. In some instances, an extractable or a leachable comprises a component of the system that is not the skin sample. Interference (volatile residuals, additives, fillers, binders, etc.) with RT-PCR test that could be present in alternative or prototype patches for this skin stripping application in some instances can be analyzed by GC-MS extraction of patch samples using solvents such as ethanol and isopropanol (which are used for RNA isolation) and quantified via the standard curve method with known concentrations of standard solutions. In some instances, methods may reflect the disclosure of WIPO Publication No. WO 2018/191268, the entire disclosure of which is incorporated herein by reference. In some instances, the method comprises one or more steps of: a) co-isolating RNA and genomic DNA from a skin sample; c) amplifying both the RNA and genomic DNA extracted from step (a); d) detecting the expression level of a RNA of interest from the RNA isolated; and/or e) detecting a mutational change, a methylation status, or a combination thereof from a gene of interest from the genomic DNA isolated. In some instances, the method comprises one or more steps of: a) contacting the biological sample obtained from an individual in need thereof with a plurality of beads; b) co-isolating RNA and genomic DNA from the plurality of beads; c) amplifying both the RNA and genomic DNA extracted from step (b); d) detecting the expression level of a RNA of interest from the RNA isolated from the beads; and/or e) detecting a mutational change, a methylation status, or a combination thereof from a gene of interest from the genomic DNA isolated from the beads. In some instances, this classification allows the quality of each patch with respect to the unnecessary extractables released to the analysis solution. In some instances, extractables are measured from a patch comprising an adhesive matrix. In some instances, the amount of extractables from a patch is about 70, 65, 60, 55, 50, 45, 40, 35, 30, 25, 20, 15, 10, or about 5 ppm per 25 cm² (3.875 square inches) area using a 20:80 IPA:H₂O extraction medium. In some instances, the amount of extractables from a patch is no more than 70, 65, 60, 55, 50, 45, 40, 35, 30, 25, 20, 15, 10, or no more than 5 ppm per 25 cm² (3.875 square inches) area using a 20:80 IPA:H₂O extraction medium. In some instances, the amount of extractables from a patch is about 700, 650, 600, 550, 500, 450, 400, 350, 300, 250, 200, 150, 100, or about 50 ppm per 25 cm² (3.875 square inches) area using an 80:20 IPA:H₂O extraction medium. In some instances, the amount of extractables from a patch is no more than 700, 650, 600, 550, 500, 450, 400, 350, 300, 250, 200, 150, 100, or no more than 50 ppm per 25 cm² (3.875 square inches) area using an 80:20 IPA:H₂O extraction medium. In some instances, the patches do not comprise a substantial amount of volatile (e.g. unreacted monomers), semi-volatile (e.g. plasticizers, process aids) or ash (e.g. inorganic fillers) type ingredients as analyzed by TGA (Thermogravimetric Analysis) TA Q50 TGA instrument. In some instances, TGA data is analyzed using TA Universal Analysis Software (no significantly measurable fillers, binders or catalysts). In some instances, unreacted monomers, semi-volatile (e.g. plasticizers, process aids) or ash (e.g. inorganic fillers) levels are below a detection limit of about 50, 40, 30, 25, 20, 18, 15, 13, 10, 8, 6, 5, or 3 ug/L of GC-MS. In some instances, BHT (butylated hydroxytoluene) levels are below a detection limit of about 50, 40, 30, 25, 20, 18, 15, 13, 10, 8, 6, 5, or 3 ug/L of GC-MS. In some instances, an amount of extractables and leachables released from the at least one adhesive patch is no more than 1.0, 1.5, 2.0, 2.5, 3.0, 3.5, 4.0, 4.5, 5.0, 5.5, or no more than 6.0, mg/cm² when at least about 25 cm² of adhesive patch is refluxed for about 3 hours in 80% ethanol.

In some embodiments, isolated RNA from a collected skin sample is reverse transcribed into cDNA for amplification by PCR to enrich for target genes. In some instances, expression levels of one or more target genes are quantified by quantitative PCR in a gene expression test. A gene expression test can provide information on a gene expression signature associated with a disease. A pigmented lesion assay is an exemplary gene expression test which measures the expression levels of target genes from RNA isolated using the adhesive skin sample collection kit.

For example, in some embodiments, the pigmented lesion assay provides objective information on a gene expression signature associated with melanoma. This information can be used to help support a histopathologic diagnosis or to determine the need for a biopsy, thereby reducing unnecessary biopsy procedures. The development of invasive tumor properties is also controlled by gene expression; therefore, the pigmented lesion assay may also differentiate invasive melanoma from melanoma in situ as well as provide staging information. The identification of invasive melanoma with metastatic potential can direct treatments to only those who need it. Another gene expression assay may determine if a melanoma tumor has spread to the lymph nodes. This test can reduce the need for a sentinel lymph node surgery, which can be extensive, cause morbidity and has significant medical costs.

Gene expression analyses can facilitate drug development by identifying drug targets and stratifying patients into groups that will maximize a drug response. In an exemplary embodiment, a skin sample collected from the face of a subject with lupus is isolated and utilized in a gene expression test to assess the expression of target genes indicated in lupus drug effects. This gene expression test can identify responders to therapy and identify new drug targets. The use of the adhesive patch allows for skin sample collection without the scarring that can occur with a biopsy.

In some embodiments, one or more polypeptides isolated from the used adhesive patch are detected and/or quantified. For example, in some embodiments, one or more polypeptides isolated from the used adhesive patch are detected and/or quantified using ELISA, immunohistochemistry, mass spectrometry, and/or absorbance measurement. In some embodiments, the sequence of DNA isolated from the used adhesive patch is determined using gene sequencing methods known to one of skill in the art.

In some instances, the skin sample collected using the patch stripping method is used in combination with other clinical assays including immunohistochemistry, mass spectrometry, immunophenotyping, fluorescent in situ hybridization (FISH), and/or any combination thereof. The skin sample does not necessarily need to be removed from the adhesive patch to prove useful as an assay component. Cellular material from the skin samples can be detected from the surface of the adhesive patch matrix. Detection methods include the use of probes configured to bind to cellular material adhered to the adhesive patch matrix. Probes include, but are not limited to, primers configured to bind to nucleic acids, and antibodies configured to bind to polypeptides, nucleic acids, small molecules, lipids, and/or carbohydrates.

In some embodiments, the patch stripping method is part of the work up for a variety of suspected skin conditions including, but not limited to, lupus, rubeola, acne, hemangioma, psoriasis, actinic keratosis, eczema, candidiasis, impetigo, shingles, leprosy and Chron's disease. Skin conditions also include atopic dermatitis, inflammatory dermatoses, bullous diseases, infections, and cancers. Skin cancers include, but are not limited to, basal cell carcinoma, actinic keratoses, merkel cell carcinoma, sebaceous carcinoma, squamous cell carcinoma, melanoma, and dermatofibrosarcoma protuberans.

In some embodiments, the patch stripping method is performed using a plurality of adhesive patches. Between 1 and 8 adhesive patches can be sequentially applied and removed to collect a skin sample. The number of adhesive patches used per skin sample may include, but is not limited to 16, 14, 12, 11, 10, 9, 8, 7, 6, 5, 4, 3, 2, 1 patches, from about 2 to about 7 patches, from about 3 to about 6 patches, and from about 4 to about 5 patches. In certain instances, an adhesive patch is applied to the skin and removed from the skin about 1 to about 8 times, e.g., sequentially or serially.

In some embodiments, the methods, devices, and systems provided herein involve applying an adhesive or other similar patch to the skin in a manner so that an effective or sufficient amount of a tissue, such as a skin sample, adheres to the adhesive matrix of the adhesive patch. For example, in some embodiments, the effective or sufficient amount of a skin sample is an amount that removably adheres to a material, such as the matrix or adhesive patch. The adhered skin sample, in certain embodiments, comprises cellular material including nucleic acids and proteins (and/or polypeptides). In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effective amount of a skin sample comprises an amount of RNA sufficient to perform a gene expression analysis. Sufficient amounts of RNA include picogram, nanogram, and microgram quantities. In some instances, the amount of cellular material or nucleic acids is measured per kit, per patch (or patches), or as a function of the surface area of the adhesive area of the patch (or patches).

The amount of cellular material collected may be measured per collection kit. In some instances, a collection kit comprises one or more patches. In some embodiments, the adhered skin sample comprises cellular material including nucleic acids such as RNA or DNA, or a polypeptide such as a protein, in an amount that is at least about 1 picogram per collection kit. In some embodiments, the amount of cellular material is no more than about 1 nanogram per collection kit. In further or additional embodiments, the amount of cellular material is no more than about 1 microgram per collection kit. In still further or additional embodiments, the amount of cellular material is no more than about 1 gram per collection kit. In further or additional embodiments, the amount of cellular material, including nucleic acids such as RNA or DNA, or a polypeptide such as a protein, is less than about 1 gram, is less than about 500 milligrams, is less than about 490 milligrams, is less than about 480 milligrams, is less than about 470 milligrams, is less than about 460 milligrams, is less than about 450 milligrams, is less than about 440 milligrams, is less than about 430 milligrams, is less than about 420 milligrams, is less than about 410 milligrams, is less than about 400 milligrams, is less than about 390 milligrams, is less than about 380 milligrams, is less than about 370 milligrams, is less than about 360 milligrams, is less than about 350 milligrams, is less than about 340 milligrams, is less than about 330 milligrams, is less than about 320 milligrams, is less than about 310 milligrams, is less than about 300 milligrams, is less than about 290 milligrams, is less than about 280 milligrams, is less than about 270 milligrams, is less than about 260 milligrams, is less than about 250 milligrams, is less than about 240 milligrams, is less than about 230 milligrams, is less than about 220 milligrams, is less than about 210 milligrams, is less than about 200 milligrams, is less than about 190 milligrams, is less than about 180 milligrams, is less than about 170 milligrams, is less than about 160 milligrams, is less than about 150 milligrams, is less than about 140 milligrams, is less than about 130 milligrams, is less than about 120 milligrams, is less than about 110 milligrams, is less than about 100 milligrams, is less than about 90 milligrams, is less than about 80 milligrams, is less than about 70 milligrams, is less than about 60 milligrams, is less than about 50 milligrams, is less than about 40 milligrams, is less than about 30 milligrams, is less than about 20 milligrams, is less than about 10 milligrams, is less than about 5 milligrams, or is less than about 1 milligram. In further or additional embodiments, the amount of cellular material is from about 1 picogram to about 1 gram per collection kit. In further or additional embodiments, the amount of cellular material is from about 1 picogram to about 1 milligram per collection kit. In further or additional embodiments, the amount of cellular material is from about 1 picogram to about 1 microgram per collection kit. In further or additional embodiments, the amount of cellular material is from about 1 picogram to about 1 nanogram per collection kit. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms per collection kit. In further or additional embodiments, the amount of cellular material, including nucleic acids such as RNA or DNA, or a polypeptide such as a protein, comprises an amount that is from about 50 microgram to 1 milligram, 50 microgram to 50 milligrams, 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms per collection kit. In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram per collection kit. In further or additional embodiments, the amount of cellular material is from about 1 picogram to about 50 microgram per collection kit. In further or additional embodiments, the cellular material comprises an amount that is from about 1 picogram to about 15 micrograms, about 1 picogram to about 10 micrograms, about 1 picogram to about 50 micrograms, about 1 picogram to about 50 micrograms, about 1 picogram to about 100 micrograms, about 1 picogram to about 200 micrograms, about 1 picogram to about 500 micrograms, about 1 picogram to about 750 micrograms, 50 picogram to about 1 microgram, from about 500 picogram to 1 microgram, from about 100 picograms to about 500 microgram, from about 500 picograms to about 100 microgram, from about 750 picograms to about 1 microgram, from about 1 nanogram to about 750 microgram, from about 100 nanogram to about 500 microgram, or from about 1 nanogram to about 500 microgram per collection kit. In further or additional embodiments, the amount of cellular material is from about 1 picogram to about 1 gram per collection kit. In further or additional embodiments, the cellular material comprises an amount that is from about 50 microgram to about 1 milligram, from about 500 microgram to 1 milligram, from about 100 picograms to about 500 milligram, from about 500 picograms to about 100 milligram, from about 750 picograms to about 1 milligram, from about 1 nanogram to about 750 milligram, or from about 1 nanogram to about 500 milligram per collection kit.

The amount of cellular material collected may be measured per area of the adhesive region of a patch. In some embodiments, the adhered skin sample comprises cellular material including nucleic acids such as RNA or DNA, or a polypeptide such as a protein, in an amount that is at least about 1 picogram per square inch. In some embodiments, the amount of cellular material is no more than about 1 nanogram per square inch. In further or additional embodiments, the amount of cellular material is no more than about 1 microgram per square inch. In still further or additional embodiments, the amount of cellular material is no more than about 1 gram per square inch. In further or additional embodiments, the amount of cellular material is from about 1 picogram to about 1 gram per square inch. In further or additional embodiments, the amount of cellular material is from about 1 picogram to about 1 milligram per square inch. In further or additional embodiments, the amount of cellular material is from about 1 picogram to about 1 microgram per square inch. In further or additional embodiments, the amount of cellular material is from about 1 picogram to about 1 nanogram per square inch. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms per square inch. In further or additional embodiments, the amount of cellular material, including nucleic acids such as RNA or DNA, or a polypeptide such as a protein, comprises an amount that is from about 50 microgram to 1 milligram, 50 microgram to 50 milligrams, 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms per square inch. In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram per square inch. In further or additional embodiments, the amount of cellular material is from about 1 picogram to about 50 microgram per square inch. In further or additional embodiments, the cellular material comprises an amount that is from about 1 picogram to about 15 micrograms, about 1 picogram to about 10 micrograms, about 1 picogram to about 50 micrograms, about 1 picogram to about 50 micrograms, about 1 picogram to about 100 micrograms, about 1 picogram to about 200 micrograms, about 1 picogram to about 500 micrograms, about 1 picogram to about 750 micrograms, 50 picogram to about 1 microgram, from about 500 picogram to 1 microgram, from about 100 picograms to about 500 microgram, from about 500 picograms to about 100 microgram, from about 750 picograms to about 1 microgram, from about 1 nanogram to about 750 microgram, from about 100 nanogram to about 500 microgram, or from about 1 nanogram to about 500 microgram per square inch. In further or additional embodiments, the amount of cellular material is from about 1 picogram to about 1 gram per square inch. In further or additional embodiments, the cellular material comprises an amount that is from about 50 microgram to about 1 milligram, from about 500 microgram to 1 milligram, from about 100 picograms to about 500 milligram, from about 500 picograms to about 100 milligram, from about 750 picograms to about 1 milligram, from about 1 nanogram to about 750 milligram, or from about 1 nanogram to about 500 milligram per square inch.

The amount of cellular material collected may be measured per patch (or patches). In some instances, a kit comprises one or more patches. In some embodiments, the adhered skin sample comprises cellular material including nucleic acids such as RNA or DNA, or a polypeptide such as a protein, in an amount that is at least about 1 picogram per patch. In some embodiments, the amount of cellular material is no more than about 1 nanogram per patch. In further or additional embodiments, the amount of cellular material is no more than about 1 microgram per patch. In still further or additional embodiments, the amount of cellular material is no more than about 1 gram per patch. In further or additional embodiments, the amount of cellular material is from about 1 picogram to about 1 gram per patch. In further or additional embodiments, the amount of cellular material is from about 1 picogram to about 1 milligram per patch. In further or additional embodiments, the amount of cellular material is from about 1 picogram to about 1 microgram per patch. In further or additional embodiments, the amount of cellular material is from about 1 picogram to about 1 nanogram per patch. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms per patch. In further or additional embodiments, the amount of cellular material, including nucleic acids such as RNA or DNA, or a polypeptide such as a protein, comprises an amount that is from about 50 microgram to 1 milligram, 50 microgram to 50 milligrams, 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms per patch. In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram per patch. In further or additional embodiments, the amount of cellular material is from about 1 picogram to about 50 microgram per patch. In further or additional embodiments, the cellular material comprises an amount that is from about 1 picogram to about 15 micrograms, about 1 picogram to about 10 micrograms, about 1 picogram to about 50 micrograms, 50 picogram to about 1 microgram, about 1 picogram to about 50 micrograms, about 1 picogram to about 100 micrograms, about 1 picogram to about 200 micrograms, about 1 picogram to about 500 micrograms, about 1 picogram to about 750 micrograms, 500 picogram to 1 microgram, from about 100 picograms to about 500 microgram, from about 500 picograms to about 100 microgram, from about 750 picograms to about 1 microgram, from about 1 nanogram to about 750 microgram, from about 100 nanogram to about 500 microgram, or from about 1 nanogram to about 500 microgram per patch. In further or additional embodiments, the amount of cellular material is from about 1 picogram to about 1 gram per patch. In further or additional embodiments, the cellular material comprises an amount that is from about 50 microgram to about 1 milligram, from about 500 microgram to 1 milligram, from about 100 picograms to about 500 milligram, from about 500 picograms to about 100 milligram, from about 750 picograms to about 1 milligram, from about 1 nanogram to about 750 milligram, or from about 1 nanogram to about 500 milligram per patch.

### Analysis of Cellular Material and Communication of Results

Cellular material may be further processed for analysis of target analytes therein. In some instances, target analytes comprise nucleic acids and proteins. In some instances, nucleic acids comprise DNA and/or RNA. In some instances, nucleic acids comprise genomic DNA. In some instances, nucleic acids comprise cDNA. In some instances, nucleic acids are of human origin. In some instances, nucleic acids are of microbial origin.

In some embodiments, isolated RNA from a collected skin sample is reverse transcribed into cDNA, for example for amplification by PCR to enrich for target genes. The expression levels of these target genes may be quantified by quantitative PCR in a gene expression test. In some instances, in combination with quantitative PCR, a software program performed on a computer is utilized to quantify RNA isolated from the collected skin sample. In some instances, a software program or module is utilized to relate a quantity of RNA from a skin sample to a gene expression signature, wherein the gene expression signature is associated with a disease such as melanoma. In some embodiments, a software program or module scores a sample based on gene expression levels. In some embodiments, the sample score is compared with a reference sample score to determine if there is a statistical significance between the gene expression signature and a disease.

In some embodiments, one or more target genes from the isolated RNA obtained from a collected skin sample are analyzed. In some instances, from about 1 to about 100, from about 1 to about 90, from about 1 to about 80, from about 1 to about 70, from about 1 to about 60, from about 1 to about 50, from about 1 to about 40, from about 1 to about 30, from about 1 to about 20, from about 5 to about 100, from about 5 to about 80, from about 5 to about 60, from about 5 to about 40, from about 5 to about 20, from about 10 to about 100, from about 10 to about 80, from about 10 to about 60, from about 10 to about 40, from about 20 to about 80, from about 20 to about 60, from about 20 to about 40, from about 30 to about 80, from about 30 to about 60, from about 40 to about 60, from about 2 to about 10, from about 2 to about 8, or from about 2 to about 6 target genes from the isolated RNA obtained from a collected skin sample are analyzed.

In some cases, about 1, 2, 3, 4, 5, 6, 7, 8, 9, 10, 11, 12, 13, 14, 15, 20, 25, 30, 40, 50 or more target genes from the isolated RNA obtained from a collected skin sample are analyzed. In some cases, about 1 or more target genes from the isolated RNA obtained from a collected skin sample are analyzed. In some cases, about 2 or more target genes from the isolated RNA obtained from a collected skin sample are analyzed. In some cases, about 3 or more target genes from the isolated RNA obtained from a collected skin sample are analyzed. In some cases, about 4 or more target genes from the isolated RNA obtained from a collected skin sample are analyzed. In some cases, about 5 or more target genes from the isolated RNA obtained from a collected skin sample are analyzed. In some cases, about 6 or more target genes from the isolated RNA obtained from a collected skin sample are analyzed. In some cases, about 7 or more target genes from the isolated RNA obtained from a collected skin sample are analyzed. In some cases, about 8 or more target genes from the isolated RNA obtained from a collected skin sample are analyzed. In some cases, about 9 or more target genes from the isolated RNA obtained from a collected skin sample are analyzed. In some cases, about 10 or more target genes from the isolated RNA obtained from a collected skin sample are analyzed. In some cases, about 11 or more target genes from the isolated RNA obtained from a collected skin sample are analyzed. In some cases, about 12 or more target genes from the isolated RNA obtained from a collected skin sample are analyzed. In some cases, about 13 or more target genes from the isolated RNA obtained from a collected skin sample are analyzed. In some cases, about 14 or more target genes from the isolated RNA obtained from a collected skin sample are analyzed. In some cases, about 15 or more target genes from the isolated RNA obtained from a collected skin sample are analyzed. In some cases, about 20 or more target genes from the isolated RNA obtained from a collected skin sample are analyzed. In some cases, about 25 or more target genes from the isolated RNA obtained from a collected skin sample are analyzed. In some cases, about 30 or more target genes from the isolated RNA obtained from a collected skin sample are analyzed. In some cases, about 40 or more target genes from the isolated RNA obtained from a collected skin sample are analyzed. In some cases, about 50 or more target genes from the isolated RNA obtained from a collected skin sample are analyzed.

In some embodiments, one or more target genes from the isolated DNA obtained from a collected skin sample are analyzed (e.g., for genomic mutations). In some instances, from about 1 to about 100, from about 1 to about 90, from about 1 to about 80, from about 1 to about 70, from about 1 to about 60, from about 1 to about 50, from about 1 to about 40, from about 1 to about 30, from about 1 to about 20, from about 5 to about 100, from about 5 to about 80, from about 5 to about 60, from about 5 to about 40, from about 5 to about 20, from about 10 to about 100, from about 10 to about 80, from about 10 to about 60, from about 10 to about 40, from about 20 to about 80, from about 20 to about 60, from about 20 to about 40, from about 30 to about 80, from about 30 to about 60, from about 40 to about 60, from about 2 to about 10, from about 2 to about 8, or from about 2 to about 6 target genes from the isolated DNA obtained from a collected skin sample are analyzed.

In some cases, about 1, 2, 3, 4, 5, 6, 7, 8, 9, 10, 11, 12, 13, 14, 15, 20, 25, 30, 40, 50 or more target genes from the isolated DNA obtained from a collected skin sample are analyzed (e.g., for genomic mutations). In some cases, about 1 or more target genes from the isolated DNA obtained from a collected skin sample are analyzed. In some cases, about 2 or more target genes from the isolated DNA obtained from a collected skin sample are analyzed. In some cases, about 3 or more target genes from the isolated DNA obtained from a collected skin sample are analyzed. In some cases, about 4 or more target genes from the isolated DNA obtained from a collected skin sample are analyzed. In some cases, about 5 or more target genes from the isolated DNA obtained from a collected skin sample are analyzed. In some cases, about 6 or more target genes from the isolated DNA obtained from a collected skin sample are analyzed. In some cases, about 7 or more target genes from the isolated DNA obtained from a collected skin sample are analyzed. In some cases, about 8 or more target genes from the isolated DNA obtained from a collected skin sample are analyzed. In some cases, about 9 or more target genes from the isolated DNA obtained from a collected skin sample are analyzed. In some cases, about 10 or more target genes from the isolated DNA obtained from a collected skin sample are analyzed. In some cases, about 11 or more target genes from the isolated DNA obtained from a collected skin sample are analyzed. In some cases, about 12 or more target genes from the isolated DNA obtained from a collected skin sample are analyzed. In some cases, about 13 or more target genes from the isolated DNA obtained from a collected skin sample are analyzed. In some cases, about 14 or more target genes from the isolated DNA obtained from a collected skin sample are analyzed. In some cases, about 15 or more target genes from the isolated DNA obtained from a collected skin sample are analyzed. In some cases, about 20 or more target genes from the isolated DNA obtained from a collected skin sample are analyzed. In some cases, about 25 or more target genes from the isolated DNA obtained from a collected skin sample are analyzed. In some cases, about 30 or more target genes from the isolated DNA obtained from a collected skin sample are analyzed. In some cases, about 40 or more target genes from the isolated DNA obtained from a collected skin sample are analyzed. In some cases, about 50 or more target genes from the isolated DNA obtained from a collected skin sample are analyzed.

Provided herein are target genes and gene classifiers for non-invasively diagnosing or detecting melanoma that may be used in combination with the methods and systems of skin or tissue sample collection disclosed. Systems and methods may reflect the disclosure of WIPO Publication No. WO 2009/140550, the entire disclosure of which is incorporated herein by reference. In some embodiments, the one or more target genes comprise C6orf218, preferentially expressed antigen in melanoma (PRAME), or a combination thereof. In some cases, the target genes comprise C6orf218. In other cases, the one or more target genes comprise preferentially expressed antigen in melanoma (PRAME). In some cases, about 1, 2, 3, 4, 5, 6, 7, 8, 9, 10, 11, 12, 13, 14, 15, 20, 25, 30, 40, 50 or more target genes from the isolated RNA, DNA or RNA/DNA obtained from a collected skin sample are analyzed, in which the one or more target genes comprise at least C6orf218, preferentially expressed antigen in melanoma (PRAME), IL-6, IL-8, IL-17A, IL-17C, IL-17F, IL-17RA, IL-17RC, IL-21, IL-22, IL-23A, IL-24, IL-26, TNF-α, TNF RSF1A, S100A7, S100A9, CCL20, CXCL1, CXCL5, LCN2, DEFB4A, or a combination thereof.

Provided herein are target genes and gene classifiers for non-invasively diagnosing or detecting non-melanoma skin cancers that may be used in combination with the methods and systems of skin or tissue sample collection disclosed. Systems and methods may reflect the disclosure of WIPO Publication No. WO 2019/161126, the entire disclosure of which is incorporated herein by reference. In some instances, the target genes comprise IGFL1, MMP1, COL5A2, IL24, AADACL2, PTCH1, CD68, PRKACA, and SPP1. In some instances, the target genes comprise MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, the non-melanoma skin cancer comprises BCC, SCC, actinic keratosis (AK), or seborrheic keratosis (SK).

Provided herein are target genes and gene classifiers for non-invasively diagnosing or detecting autoimmune or inflammatory disorders that may be used in combination with the methods and systems of skin or tissue sample collection disclosed. Systems and methods may reflect the disclosure of WIPO Publication No. WO 2019/217478, the entire disclosure of which is incorporated herein by reference. In some instances, the disorder comprises psoriasis, atopic dermatitis, or lupus. In some instances, the one or more target genes comprise one or more of IL-17A, IL-17F, IL-8, CXCL5, S100A9, DEFB4A, or a combination thereof. In some instances, the one or more target genes comprises IL-17C, S100A7, IL-17RA, IL-17RC, IL-23A, IL-22, IL-26, IL-24, IL-6, CXCL1, TNFa, LCN2, CCL20, INFRSF1A, or a combination thereof. In some instances, the one or more target genes comprises L-17C, S100A7, IL-17RA, IL-17RC, IL-23A, IL-22, IL-26, 11. -24, IL-6, CXCU, IFN-gamma, 11-3/, IL-33, TNFa, LCN2, CCL20, TNFRSF1A, or a combination thereof. In some instances, the one or more target genes comprises a gene in the Th1, Th2, Th17, or Th22 pathway. In some instances, the target genes comprise IL-13, IL-31, TSLP, IL-13R, IL-4R, IL-17, IL-22, CXCL9, CXCL10, CXCLH, S100A7, S100A8, S100A9, CCL17, CCL18, CCL19, CCL26, CCL27, NOS2, IL-31RA, CCL17, IL-23A, IL-4R, IL-22, IL-13, or IL-13RA1, IL-13 pathway constituents or receptors, or a combination thereof.

Provided herein are target genes and gene classifiers for non-invasively diagnosing or detecting skin cancers that may be used in combination with the methods and systems of skin or tissue sample collection disclosed. Systems and methods may reflect the disclosure of WIPO Publication No. WO 2020/198229, the entire disclosure of which is incorporated herein by reference. In some instances, the skin cancer comprises cutaneous T cell lymphoma (CTCL). In some cases, the skin cancer is mycosis fungoides (MF) or Sézary syndrome (SS). In some instances, the at least one target gene comprises a gene encoding a saposin-like protein, a gene encoding a FYN-binding protein family member, a gene encoding a TEC kinase family member, a gene encoding a STAT, a gene encoding a TRAF3 interacting protein, a gene encoding a CXC chemokine family member, or a combination thereof. In some instances, the target genes comprise FYB, PK, IL26, STAT5A, TRAF3IP3, ONLY, DNM3, TNFSF11, TOX, LEF1, CCR4, POU2AF1, GTSF1, PLS3, MMP12, LCK, NEDD4L, or a combination thereof. In some embodiments, the at least one target gene comprises FYN binding protein (FYB), IL2 inducible T-cell kinase (ITK), interleukin 26 (IL26), signal transducer and activator of transcription 5 A (STATSA), TRAF3 interacting protein 3 (RAF3IP3), granulysin (GNLY), dynamin 3 (DNM3), or tumor necrosis factor superfamily member 11 (TNFSF11), or a combination thereof. In some embodiments, the at least one target gene comprises TOX, LEF1, CCR4, POU2AF1, GTSF1, PLS3, MMP12, ZCX, or NEDD4L, or a combination thereof. In some embodiments, the at least one target gene comprises FYB, GNLY, PK, STAT5, TRAF3IP3, CXCL10, CXCL8, or 77VF, or a combination thereof. In some embodiments, the at least one target gene comprises a gene encoding a microRNA. In some embodiments, the microRNA comprises miR-21, miR-29b, miR-155, miR-186, miR-214, or miR-221. Some embodiments include detecting the presence of at least one genotype of target genes known to be mutated in subjects with CTCL, in the nucleic acids or in a separate set of nucleic acids isolated from the skin sample. In some embodiments, determining whether the subject has CTCL further comprises determining whether the subject has CTCL based on the presence of the at least one genotype. In some embodiments, the target genes comprise TP53, ZEB1, ARID A, DNMT3A, CDKN2A, FAS, STATSB, PRKCQ, RHOA, DNMT3A, PLCG1, or NFKB2.

Provided herein are target genes and gene classifiers related to UV skin damage that may be used in combination with the methods and systems of skin or tissue sample collection disclosed. Systems and methods may reflect the disclosure of WIPO Publication No. WO 2020/206085, the entire disclosure of which is incorporated herein by reference. In some instances, target genes comprise ADAMTSL4, CDKN1A, CDKN2A, CST6, KIF18B, MKI67, SLAMF7, TRIP13, UHRF1, CRABP2, ILIRN, IL22RA1, IL36B, IL36G, KLK10, KRT17, MUCL1, PDCD4, SPRR1A, or a combination thereof.

Provided herein are systems and methods of assessing mutation burden in skin that may be used in combination with the methods and systems of skin or tissue sample collection disclosed. In some instances, mutation burden is measured by genomic analysis. In some instances, the mutation burden indicates an extent of UV damage, aging, or exposure to environmental mutagens. Systems and methods may reflect the disclosure of U.S. Patent No. 63/117,946, the entire disclosure of which is incorporated herein by reference. In some instances, a sample comprises a majority of skin sampled from a layer of skin exposed to an environmental factor. In some instances, the environmental factor is ultraviolet (UV) light. In some instances, the number of nucleic acid mutations per mm² of skin collected comprises at least 1, 2, 5, 10, 15, 20, 25, 30, 4, or at least 50 mutations. In some instances, the at least one nucleic acid mutation is indicative of UV damage. In some instances, target genes comprise TP53, NOTCH1, NOTCH2, NOTCH3, RBM10, PPP2R1A, GNAS, CTNNB1, PIK3CA, PPP6C, HRAS, KRAS, MTOR, SMAD3, LMNA, FGFR3, ZNF750, EPAS1, RPL22, ALDH2, CBFA2T3, CCND1, FAT1, FH, KLF4, CIC, RAC1, PTCH1, TPM4, or a combination thereof.

Skin samples obtained from the non-invasive methods and systems described herein may comprise non-human cellular material and/or nucleic acids. In some instances, samples comprise microorganisms. In some instances, samples comprise microbial cells or cellular material, proteins or protein subunits, nucleic acids, or nucleic acid fragments from fungi, protozoa, bacteria (Gram positive or Gram negative), yeast, virus, parasite, or other non-human microorganisms. In some instances, methods and systems described herein are used to characterize a skin microbiome. In some instances, the skin microbiome is analyzed to determine the presence of infection. In some instances, the skin microbiome is analyzed to determine general skin health. In one embodiment, a skin microbiome indicative of increased likelihood to develop a metabolic syndrome or a condition associated therewith comprises reduced bacterial community diversity, e.g., reduced number of different bacterial species, strains, or both. In one embodiment, determining that a skin microbiome comprises determining abundance of a species belonging to any family selected from: Streptococcaceae, Corynebacteriaceae, Staphylococcaceae, Micrococcaceae, Neisseriaceae, Pasteurellaceae, Prevotellaceae, Brevibacterium, Dermabacter, Malasezzia, and Moraxellaceae, ratio of two or more species belonging to any one of the aforementioned families, or both. In some embodiments, a skin microbiome is indicative of increased likelihood to develop a disease or a condition. In some instances, the disease or condition is a metabolic disease or condition. In some instances, the microorganism comprises one or more of Streptococcaceae, Staphylococcaceae, Micrococcaceae, Neisseriaceae, Pasteurellaceae, Brevibacterium, Dermabacter, Malasezzia, and Moraxellaceae. In some instances, the microorganism comprises one or more of Corynebacterium (e.g., C. kroppenstedtii) colonization, Staphylococcus, (e.g., S. aureus, S. epidermidis colonization, S. hominis colonization), or any combination thereof. In another embodiment, a skin microbiome indicative of increased likelihood to develop the metabolic syndrome or a condition associated therewith comprises colonization of one or more bacteria belonging to any family selected from: Streptococcaceae, Corynebacteriaceae, Staphylococcaceae, Micrococcaceae, Neisseriaceae, Pasteurellaceae, Prevotellaceae, Brevibacterium, Dermabacter, Malasezzia, and Moraxellaceae. In another embodiment, a skin microbiome indicative of increased likelihood to develop the metabolic syndrome or a condition associated therewith comprises Corynebacterium colonization. In another embodiment, a skin microbiome indicative of increased likelihood to develop the metabolic syndrome or a condition associated therewith comprises Staphylococcus aureus colonization. In another embodiment, a skin microbiome indicative of increased likelihood to develop the metabolic syndrome or a condition associated therewith comprises high Corynebacterium kroppenstedtii colonization. In another embodiment, a skin microbiome indicative of increased likelihood to develop the metabolic syndrome or a condition associated therewith comprises high Staphylococcus aureus colonization. In another embodiment, a skin microbiome indicative of increased likelihood to develop the metabolic syndrome or a condition associated therewith comprises increased Corynebacterium, (e.g., C. kroppenstedtii, colonization), increased Staphylococcus, (e.g., S. aureus colonization, reduced S. epidermidis colonization, reduced S. hominis colonization), or any combination thereof. In another embodiment, a skin microbiome indicative of increased likelihood to develop the metabolic syndrome or a condition associated therewith comprises colonization of one or more bacteria belonging to any family selected from: Streptococcaceae, Corynebacteriaceae, Staphylococcaceae, Micrococcaceae, Neisseriaceae, Pasteurellaceae, Prevotellaceae, Brevibacterium, Dermabacter, Malasezzia, and Moraxellaceae. In another embodiment, a skin microbiome indicative of increased likelihood to develop the metabolic syndrome or a condition associated therewith comprises Corynebacterium colonization. In another embodiment, a skin microbiome indicative of increased likelihood to develop the metabolic syndrome or a condition associated therewith comprises Staphylococcus aureus colonization. In another embodiment, a skin microbiome indicative of increased likelihood to develop the metabolic syndrome or a condition associated therewith comprises high Corynebacterium kroppenstedtii colonization. In another embodiment, a skin microbiome indicative of increased likelihood to develop the metabolic syndrome or a condition associated therewith comprises high Staphylococcus aureus colonization. In another embodiment, a skin microbiome indicative of increased likelihood to develop the metabolic syndrome or a condition associated therewith comprises increased Corynebacterium, e.g., (C. kroppenstedtii) colonization, increased Staphylococcus, (e.g., S. aureus colonization, reduced S. epidermidis colonization, reduced S. hominis colonization), or any combination thereof. In some instances, a microorganism detected using the non-invasive sampling systems and methods described herein comprises one or more of Staphylococcus epidermidis, Staphylococcus aureus, Staphylococcus warneri, Streptococcus pyogenes, Streptococcus mitis, Cutibacterium acnes, Corynebacterium spp., Acinetobacter johnsonii, and Pseudomonas aeruginosa. In some instances, a microorganism detected using the non-invasive sampling systems and methods described herein comprises one or more of Candida albicans, Rhodotorula rubra, Torulopsis and Trichosporon cutaneum, dermatophytes (skin living fungi) such as Microsporum gypseum, and Trichophyton rubrum and nondermatophyte fungi (opportunistic fungi that can live in skin) such as Rhizopus stolonifer, Trichosporon cutaneum, Fusarium, Scopulariopsis brevicaulis, Curvularia, Alternaria alternata, Paecilomyces, Aspergillus flavus and Penicillium.

The subject matter described herein, including the gene expression tests and corresponding transmission of data, in certain aspects, are configured to be performed in one or more facilities at one or more locations. Facility locations are not limited by country and include any country or territory. Facility locations are not limited by country and include any country or territory. In some instances, one or more steps of the gene expression test are performed in a different country than another step of the gene expression test. In some instances, one or more steps of the gene expression test are performed in a different country than one or more steps of the patch stripping aspect. In some embodiments, one or more articles are transferred from one or more of the facilities to one or more different facilities for analysis or further analysis. An article includes, but is not limited to, one or more components of the skin sample collection kit, a used adhesive patch, isolated cellular material obtained from a used adhesive patch, processed cellular material, and/or data. Processed cellular material includes, but is not limited to, cDNA reverse transcribed from RNA, amplified RNA, and amplified cDNA. Data includes, but is not limited to, information regarding the expression level of one or more target genes, information regarding a gene expression signature, and information regarding a disease, such as melanoma. In some embodiments of the methods, devices, and systems described herein, the analysis is performed, and a subsequent data transmission step will convey or transmit the results of the analysis. Information regarding a disease, includes, but is not limited to, identification of a disease state, likelihood of treatment success for a given disease state, type of treatment, identification of progression of a disease state (e.g., invasiveness of melanoma), and identification of a disease stage (e.g., melanoma stages 0, 1, 2, 3, or 4).

In certain examples, the application of the adhesive patch to a skin sample comprises holding the skin taut and pressing the adhesive patch firmly on the skin surface while making circular motions on the patch. Between about 1 and about 20, between about 1 and about 15, between about 1 and about 10, between about 1 and about 5, between about 5 and about 20, between about 10 and about 20, and between about 10 and 15 circular motions are made on the patch. In one embodiment, about 15 circular motions are made on the patch. In some embodiments, the patch is configured to remain on the skin surface for up to 6, 5, 4, 3, 2, and 1 minutes. After firm application to the skin, the patch is slowly removed in one direction. In certain aspects, the patch stripping method further comprises demarcating the sampled skin region on a second surface of a transparent adhesive patch, wherein the first surface is the skin facing surface comprising the adhesive matrix. The demarcation indicates the sample region to be processed. The demarcation may be the outline of a skin lesion. The marker used for demarcation may be provided in the skin sample collection kit.

In some embodiments of the subject matter described herein, the adhesive skin sample collection kit comprises a self-addressed package for delivery of one or more used adhesive patches to a facility. In some instances, the package includes a prepaid shipping label. In some embodiments, the facility is a facility which will perform one or more diagnostic steps or procedures involving the cellular material adhered to the one or more used adhesive patches. In some embodiments, the one or more diagnostic procedures includes, but is not limited to, any step performed in a gene expression test (e.g., a pigmented lesion assay), immunohistochemistry assay, immunophenotyping, ELISA, fluorescent in situ hybridization (FISH), and/or gene sequencing. The facility where any diagnostic procedure or patch stripping method described herein is performed is not limited to one country. In some instances, one or more diagnostic procedures or patch stripping methods are performed in one or more different countries. In some embodiments, a diagnostic procedure includes data analysis for any step of any diagnostic procedure described herein. In some embodiments, any step of any diagnostic procedure described herein is performed by a software program or module on a computer. In additional or further embodiments, data from any step of any procedure described herein is transferred to and from facilities located within the same or different countries, including analysis performed in one facility in a particular location and the data shipped to another location or directly to an individual in the same or a different country. In additional or further embodiments, data from any step of any procedure described herein (including analysis of cellular material such as DNA, RNA, and protein as well as transformed data from cellular material) is transferred to and/or received from a facility located within the same or different countries, including analysis of a data input, such as cellular material, performed in one facility in a particular location and corresponding data transmitted to another location, or directly to an individual, such as data related to the diagnosis, prognosis, responsiveness to therapy, or the like, in the same or different location or country.

The adhesive skin sample collection kit may be configured so that the patch stripping method is performed by a variety of operators in a variety of locations. In some embodiments, the method is performed in a clinician's office, an outpatient facility or at a home. The method is not limited to use in a facility and is configured to be utilized in a variety of locales. The method may be performed by a practitioner, nurse or any individual who has read and understood the instructions for use and is capable of performing the method according to the instructions for use sheet, including the patient or subject themselves.

Provided herein are laser scanning and sampling methods and systems that may be used in combination with the methods and systems of skin or tissue sample collection disclosed. Systems and methods may reflect the disclosure of PCT Application No. PCT/US2021/028415, the entire disclosure of which is incorporated herein by reference. In some instances, a method comprises isolating cells of interest from a tissue sample collection kit. some instances, the method comprises one or more of receiving one or more sample collectors comprising cells of interest; positioning the one or more sample collectors on a substrate; imaging the one or more sample collectors to generate at least one first image; applying a software algorithm to the at least one first image to identify a delineation between the cells of interest and a surrounding portion of each sample collector; and/or cutting the cells of interest from a remaining portion of each sample collector with a cutting system based on the identified delineation.

In some instances, the skin sample collection kit is used in combination with skin condition monitoring. For example, images of the skin sample tested are captured and stored on a mobile photoinformatic platform that maintains the images with the associated clinical information and data relating to the skin lesion sampled.

Provided herein are teledermatology methods and systems methods and systems that may be used in combination with the methods and systems of skin or tissue sample collection disclosed. Systems and methods may reflect the disclosure of PCT Publication No. PCT/US2021/028415, the entire disclosure of which is incorporated herein by reference. In some instances, systems are configured for assessing a location on skin of an individual. In some instances the system comprises one or more of a first device comprising at least one processor and instructions executable by the at least one processor to provide a first application configured to perform operations comprising: accessing a camera to capture at least one photo of the individual's skin; and submitting a request for a virtual visit for a skin condition of the individual; and a second device comprising at least one processor and instructions executable by the at least one processor to provide a second application configured to perform operations comprising: receiving a notification that the virtual visit is completed by the individual; providing access to an interface for reviewing a record of the virtual visit; providing access to an interface for identifying at least one location on the individual's skin that requires further assessment; and submitting a request to send a non-invasive skin tissue sample kit to the individual.

### Computer Program

The methods, software, media, and systems disclosed herein comprise at least one computer processor, or use of the same. The computer processor may comprise a computer program. A computer program may include a sequence of instructions, executable in the digital processing device's CPU, written to perform a specified task. Computer readable instructions may be implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. A computer program may comprise one sequence of instructions. A computer program may comprise a plurality of sequences of instructions. A computer program may be provided from one location. A computer program may be provided from a plurality of locations. A computer program may include one or more software modules. A computer program may include, in part or in whole, one or more web applications, one or more mobile applications, one or more standalone applications, one or more web browser plug-ins, extensions, add-ins, or add-ons, or combinations thereof.

### Web Application

A computer program may include a web application. In light of the disclosure provided herein, those of skill in the art will recognize that a web application may utilize one or more software frameworks and one or more database systems. A web application may be created upon a software framework such as Microsoft® .NET or Ruby on Rails (RoR). A web application may utilize one or more database systems including, by way of non-limiting examples, relational, non-relational, feature oriented, associative, and XML database systems. Suitable relational database systems may include, by way of non-limiting examples, Microsoft® SQL Server, mySQL™, and Oracle®. Those of skill in the art will also recognize that a web application may be written in one or more versions of one or more languages. A web application may be written in one or more markup languages, presentation definition languages, client-side scripting languages, server-side coding languages, database query languages, or combinations thereof. A web application may be written to some extent in a markup language such as Hypertext Markup Language (HTML), Extensible Hypertext Markup Language (XHTML), or eXtensible Markup Language (XML). A web application may be written to some extent in a presentation definition language such as Cascading Style Sheets (CSS). A web application may be written to some extent in a client-side scripting language such as Asynchronous Javascript and XML (AJAX), Flash® Actionscript, Javascript, or Silverlight®. A web application may be written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. A web application may be written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash® HTML 5, Apple® QuickTime®, Microsoft® Silverlight®, Java™, and Unity®.

### Mobile Application

A computer program may include a mobile application provided to a mobile digital processing device. The mobile application may be provided to a mobile digital processing device at the time it is manufactured. The mobile application may be provided to a mobile digital processing device via the computer network described herein.

A mobile application may be created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments may be available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums may be available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

A computer program may include a standalone application, which may be a program that may be run as an independent computer process, not an add-on to an existing process, e.g., not a plug-in. Those of skill in the art will recognize that standalone applications may be often compiled. A compiler may be a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. A computer program may include one or more executable complied applications.

### Web Browser Plug-In

A computer program may include a web browser plug-in. In computing, a plug-in may be one or more software components that add specific functionality to a larger software application. Makers of software applications may support plug-ins to enable third-party developers to create abilities which extend an application, to support easily adding new features, and to reduce the size of an application. When supported, plug-ins may enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks may be available that enable development of plug-ins in various programming languages, including, by way of non-limiting examples, C++, Delphi, Java™, PHP, Python™, and VB .NET, or combinations thereof.

Web browsers (also called Internet browsers) may be software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser may be a mobile web browser. Mobile web browsers (also called microbrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. A software module may comprise a file, a section of code, a programming feature, a programming structure, or combinations thereof. A software module may comprise a plurality of files, a plurality of sections of code, a plurality of programming features, a plurality of programming structures, or combinations thereof. The one or more software modules may comprise, by way of non-limiting examples, a web application, a mobile application, and a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, or use of the same. In view of the disclosure provided herein, those of skill in the art will recognize that many databases may be suitable for storage and retrieval of geologic profile, operator activities, division of interest, and/or contact information of royalty owners. Suitable databases may include, by way of non-limiting examples, relational databases, non-relational databases, feature oriented databases, feature databases, entity-relationship model databases, associative databases, and XML databases. A database may be internet-based. A database may be web-based. A database may be cloud computing-based. A database may be based on one or more local computer storage devices.

### Embodiments

Provided herein are numbered embodiments 1-75. Embodiment 1. A system for non-invasive collection and analysis of a skin sample, the system comprising: an adhesive skin sample collection kit comprising at least one adhesive patch, wherein the least one adhesive patch comprises: a backing layer comprising a collection area; a non-adhesive handling area; and an adhesive matrix on a surface of the collection area, wherein the adhesive matrix is configured to adhere an amount of a skin sample. Embodiment 2. The system of embodiment 1, wherein one or more of the following: (a) the backing layer comprises a flexibility to conform to a morphology of a portion of skin comprising a lesion, and wherein the backing layer comprises a thickness such the at least one adhesive patch resists wrinkling when the at least one adhesive patch is released from the skin; (b) the at least one patch comprises a thickness such that it does not self-adhere when supported by a portion of the non-adhesive handling layer with a draft and in multiple orientations; (c) an amount of extractables and leachables released from the at least one adhesive patch is less about than 3.0 mg/cm² when at least about 25 cm² patch is refluxed for about 3 hours in 80% ethanol; (d) the at least one adhesive patch comprises a longest dimension of about a wrinkling wavelength of the at least one adhesive patch; and (e) the adhesive matrix comprises a pressure sensitive adhesive, wherein the pressure sensitive adhesive exhibits a glass transition temperatures lower than 5° C. Embodiment 3. The system of embodiment 2, wherein 2 or more, 3 or more, 4 or more, or 5 or more of (a), (b), (c), (d), and (e). Embodiment 4. The system of embodiment 2 or 3, wherein at least (a). Embodiment 5. The system of embodiment 4, wherein the backing layer has an elastic modulus from about 200 to about 2,000 Psi as measured by ASTM D-882. Embodiment 6. The system of embodiment 5, wherein the backing layer has an elastic modulus of from about 1000 to about 2000 Psi. 7. The system of embodiments 5 or 6, wherein the backing layer has an elastic modulus of from about 500 to about 1500 Psi. Embodiment 8. The system of any one of embodiments 4-7, wherein the backing layer has a tensile strength of from about 7 to about 60 MPa. Embodiment 9. The system of embodiment 8, wherein the backing layer has a tensile strength of from about 30 to about 60 MPa. Embodiment 10. The system of embodiments 8 or 9, wherein the backing layer has a tensile strength of from about 7 to about 15 MPa. Embodiment 11. The system of any one of embodiments 1-10, wherein at least (b). Embodiment 12. The system of embodiment 11, wherein a thickness of the backing layer is greater than about 2 mil as measured by ASTM D6988. Embodiment 13. The system of embodiment 12, wherein a thickness of the backing layer is from about 3 to about 5 mil. Embodiment 14. The system of any one of embodiments 1-13, wherein at least (c). Embodiment 15. The system of embodiment 14, wherein the amount of extractables and leachables released from the at least one adhesive patch is less about than 1.0 mg/cm2. Embodiment 16. The system of embodiment 15, wherein the amount of extractables and leachables is characterized by GC-MS. Embodiment 17. The system of embodiments 15 or 16, wherein the amount of extractables and leachables is characterized by thermogravimetric analysis. Embodiment 18. The system of any one of embodiments 14-17, wherein an extractable or a leachable comprises a component of the system that is not the skin sample. Embodiment 19. The system of embodiment 18, wherein the extractable or the leachable comprises a non-volatile material, a semi-volatile material, or ash. Embodiment 20. The system of embodiment 19, wherein the adhesive matrix comprises a polymer and wherein the non-volatile material comprises on or more monomers of the polymer. Embodiment 21. The system of embodiments 19 or 20, wherein the semi-volatile material comprises a plasticizer or a process aid. Embodiment 22. The system of any one of embodiments 14-21, wherein an extractable or a leachable comprises BHT and wherein the BHT is less than about 10 ug/L measured by GC-MS. Embodiment 23. The system of any one of embodiments 1-22, wherein at least (d). Embodiment 24. The system of embodiment 23, wherein the longest dimension is as less than about 10, about 8, about 6, about 5, about 4, or about 3 cm. Embodiment 25. The system of any one of embodiments 1-24, wherein at least (e). Embodiment 26. The system of embodiment 25, wherein the glass transition temperatures is from about −10 to about −70° C. as measured by ASTM D3418. Embodiment 27. The system of any one of embodiments 1-26, further comprising a release panel. Embodiment 28. The system of any one of embodiments 1-27, further comprising at least one placement area panels. Embodiment 29. The system of any one of embodiments 1-28, wherein the amount of the skin sample is less than about 20 milligrams, less than about 4 milligrams, or from about 1 picogram to about 2000 micrograms of cellular material. Embodiment 30. The system of embodiment 29 wherein an amount of the skin sample on each of the at least one adhesive patch is from about 1 picogram to about 500 micrograms per patch. Embodiment 31. The system of embodiments 29 or 30, wherein the system comprises a plurality of adhesive patches comprising a total amount of the skin sample, wherein the total amount is less than about 20 milligrams, about 10 milligrams, or about 5 milligrams. Embodiment 32. The system of any one of embodiments 1-31, wherein the adhesive matrix comprises a peel adhesion strength from about 1 to about 30N/inch, as measured by ASTM D3330 at a 180° peel adhesion at a pull rates from about 1.0 inch/min to about 12.0 inch/min. Embodiment 33. The system of embodiment 32, wherein the peel adhesion is from about 10 to about 20 N/inch. Embodiment 34. The system of any one of embodiments 1-33, wherein the adhesive matrix comprises one or more of an acrylic, a silicone, and a hydrocarbon rubber. Embodiment 35. The system of any one of embodiments 1-33, wherein the adhesive matrix comprises an acrylic and a hydrocarbon rubber. Embodiment 36. The system of embodiment 34 or 35, wherein the hydrocarbon rubber comprises one or more of butyl rubber, styrene-butadiene rubber, ethyl-vinyl acetate polymers, styrene-isoprene-butadiene rubbers, or combinations thereof. Embodiment 37. The system of any one of embodiments 34-36, wherein the acrylic comprises one or more of styrene, α-methyl styrene, vinyl naphthalene, vinyl toluene, chloromethyl styrene, methyl acrylate, acrylic acid, methacrylic acid, methyl methacrylate, ethyl acrylate, ethyl methacrylate, butyl acrylate, butyl methacrylate, isobutyl acrylate, isobutyl methacrylate, ethylhexyl acrylate, ethylhexyl methacrylate, lauryl methacrylate, lauryl acrylate, octyl acrylate, octyl methacrylate, glycidyl methacrylate, allyl methacrylate, vinyl methacrylate, acetoacetoxyethyl acrylate, acetoacetoxyethyl methacrylate, acetoacetoxypropyl acrylate, acetoacetoxypropyl methacrylate, hydroxybutenyl methacrylate, the allyl ester of maleic acid, the diallyl ester of maleic acid, poly(allyl glycidyl ether), alkyl crotonates, vinyl cetate, di-n-butyl maleate, di-octylmaleate, acrylonitrile, diacetone acrylamide, acrylamide, methacrylamide, hydroxyethyl methacrylate, hydroxyethyl acrylate, acrylonitrile, t-butylaminoethyl methacrylate, dimethylaminoethyl methacrylate, diethylaminoethyl methacrylate, N, N-dimethylaminopropyl methacrylamide, 2-t-butylaminoethyl methacrylate, N, N-dimethylaminoethyl acrylate, N-(2-methacryloyloxy-ethyl)ethylene urea, and methacrylamidoethylethylene urea, or combinations thereof. Embodiment 38. The system of any one of embodiments 1-37, wherein the backing layer comprises a soft, clear, and pliable synthetic polymer. Embodiment 39. The system of embodiment 38, wherein the soft, clear, and pliable synthetic polymer comprises a thermoplastic polyurethane (TPU) or low density polyethylene (LDPE). Embodiment 40. The system of embodiments 38 or 39, wherein the soft, clear, and pliable synthetic polymer comprises polyethylene terephthalate (PET), Teflon, polyimide, polyethylene naphthalate (PEN), or acetate. Embodiment 41. The system of any one of embodiments 38-40, wherein the soft, clear, and pliable synthetic polymer comprises an elastomer of olefin. Embodiment 42. The system of embodiment 41, wherein the elastomer of olefin comprises copolymers or compounds of polymers comprising one or more of ethylene, propylene, isobutylene, vinyl acetate, vinyl alcohol, ethylene oxide, and propylene oxide. Embodiment 43. The system of any one of embodiments 38-42, wherein the soft clear, and pliable synthetic polymer comprises a thermoplastic elastomer. Embodiment 44. The system of embodiment 43, wherein the thermoplastic elastomer comprises a polyester based elastomer. Embodiment 45. The system of embodiments 43 or 44, wherein the thermoplastic elastomer comprises a copolymer or compound of an ether or an amide. Embodiment 46. The system of any one of embodiments 1-45, wherein the at least one adhesive patch has a haze values less than about 30% as measured by ASTM D1003. Embodiment 47. The system of embodiment 46, wherein the haze value is less than about 15%. Embodiment 48. The system of any one of embodiments 1-45, wherein at least one of the backing layer and adhesive matrix is water soluble. Embodiment 49. The system of any one of embodiments 1-45, wherein the at least one adhesive patch is water soluble. Embodiment 50. The system of embodiment 48 or 49, wherein at least one of the backing layer and adhesive matrix is configured to dissolve during skin sample lysis. Embodiment 51. The system of any one of embodiments 48-50, wherein the adhesive matrix comprises at least 12 oz/in² loop tackiness. Embodiment 52. The system of any one of embodiments 48-51, wherein the adhesive matrix comprises a working temperature range from −40 to 176° F. Embodiment 53. The system of any one of embodiments 48-52 wherein backing layer comprises at least 20 lb/inch tensile force. Embodiment 54. The system of any one of embodiments 48-53 wherein backing layer comprises at least 200 mN tear strength. Embodiment 55. The system of any one of embodiments 48-54 wherein the adhesive patch is dissolvable in no more than 30 seconds. Embodiment 56. The system of any one of embodiments 48-55 wherein the adhesive patch has an shelf life of at least 12 months. Embodiment 57. A kit comprising the system of any one of the preceding embodiments and further comprising a packaging comprising instructions to perform one or more of the following: peel the patch slowly; and peel at an angle greater than about perpendicular to the skin surface. Embodiment 58. The kit of embodiment 57, wherein slowly is indicated as less than about 1 linear inch peeled per about five seconds. Embodiment 59. A kit comprising: at least one adhesive patch, wherein the least one adhesive patch comprises: a backing layer comprising a collection area; a non-adhesive handling area; an adhesive matrix on a surface of the collection area, wherein the adhesive matrix is configured to adhere to an amount of a skin sample; and a packaging comprising instructions to perform one or more of the following: peel the patch slowly; and peel at an angle greater than about perpendicular to the skin surface. Embodiment 60. The kit of embodiment 59, wherein slowly is indicated as less than about 1 linear inch peeled per about five seconds. Embodiment 61. The kit of embodiment 59 or 60, wherein one or more of the following: (a) the backing layer comprises a flexibility to conform to a morphology of a portion of skin comprising a lesion, and wherein the backing layer comprises a thickness such the at least one adhesive patch resists wrinkling when the at least one adhesive patch is released from the skin; (b) the at least one patch comprises a thickness such that it does not self-adhere when supported by a portion of the non-adhesive handling layer with a draft and in multiple orientations; (c) an amount of extractables and leachables released from the at least one adhesive patch is less about than 3.0 mg/cm² when at least about 25 cm² patch is refluxed for about 3 hours in 80% ethanol; (d) the at least one adhesive patch comprises a longest dimension of about a wrinkling wavelength of the at least one adhesive patch; and (e) the adhesive matrix comprises a pressure sensitive adhesive, wherein the pressure sensitive adhesive exhibits a glass transition temperatures lower than 5° C. Embodiment 62. The kit of embodiment 61, wherein 2 or more, 3 or more, 4 or more, or 5 or more of (a), (b), (c), (d), and (e). Embodiment 63. A kit for non-invasive collection and analysis of a skin sample, the kit comprising: at least one adhesive patch, wherein the least one adhesive patch comprises: a backing layer comprising a collection area; a non-adhesive handling area; an adhesive matrix on a surface of the collection area, wherein the adhesive matrix is configured to adhere to an amount of a skin sample; and a return pouch sized and shaped to receive the at least one adhesive patch, the return pouch comprising a desiccant. Embodiment 64. The kit of embodiment 63, wherein the desiccant is configured to prevent the activity of RNases in the skin sample. Embodiment 65. The kit of embodiment 63, wherein an amount of the desiccant is from about 0.5 grams to about 5 grams. Embodiment 66. The kit of embodiment 65, wherein the amount of the desiccant is about 2 grams. Embodiment 67. The kit of embodiment 63, wherein the return pouch is plastic or foil. Embodiment 68. The kit of embodiment 63, wherein the return pouch is sealable. Embodiment 69. The kit of embodiment 63, wherein the desiccant is silica gel. Embodiment 70. The kit of embodiment 63, further comprising a packaging comprising instructions to perform one or more of the following: (a) peel the patch slowly; and (b) peel at an angle greater than about perpendicular to the skin surface. Embodiment 71. The kit of embodiment 70, wherein slowly is indicated as less than about 1 linear inch peeled per about five seconds. Embodiment 72. A method for analyzing a skin sample comprising: receiving at least one adhesive patch from the system or kit of any one of embodiments 57-71; and quantifying expression levels of one or more target genes in the skin sample. Embodiment 73. The method of embodiment 72, wherein the method further comprises extracting nucleic acids from at least a portion of the skin sample. Embodiment 73. The system, method, or kit of any one of embodiments 1-72 wherein each adhesive patch collects 500-20,000 pg of nucleic acids. Embodiment 74. The system, method, or kit of any one of embodiments 1-73 wherein each adhesive patch collects 500-2000 pg of DNA. Embodiment 75. The system, method, or kit of any one of embodiments 1-74 wherein each adhesive patch collects 1000-15,000 pg of RNA.

### EXAMPLES

### Example 1: Point of Care Skin Sample Collection

A pigmented lesion located on the hand of a subject is selected for skin sampling. The skin sampling area contains a minimal amount of hair, is not irritated and has not been previously biopsied. The lesion is about 8 mm in size. As exemplified in FIG. 1, the skin sampling area (101) comprising the skin lesion (102) is cleansed with an alcohol pad (103) by a practitioner (104) wearing gloves, and the skin is allowed to air dry for 5 minutes.

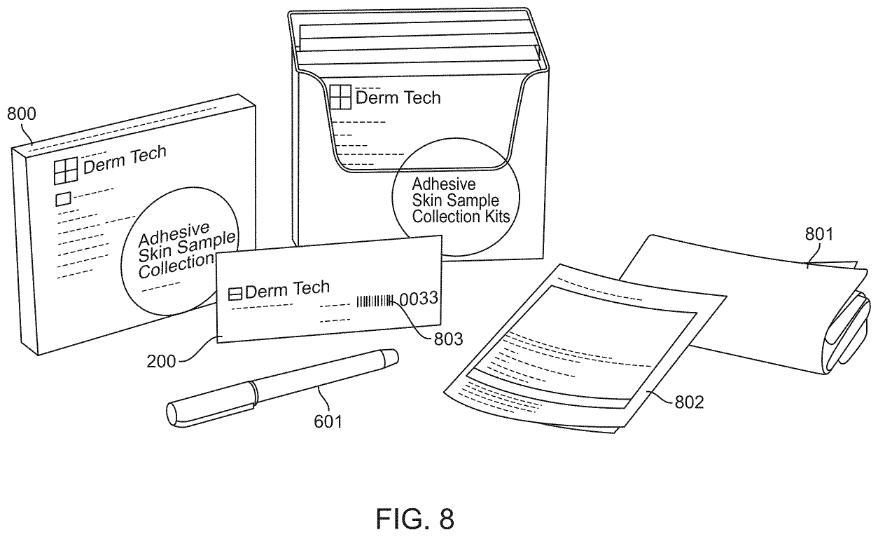

A tri-fold skin sample collector is removed from an adhesive skin sample collection kit exemplified by FIG. 8. FIG. 2 exemplifies the tri-fold skin sample collector (200) comprising a peelable release panel (201) comprising four adhesive patches (202), a placement area panel (203) comprising a removable liner (204), and a clear panel (205). The tri-fold skin sample collector has a barcode specific for the subject. The removable liner is removed from the placement area panel (203), exposing four regions (206) designated for the placement of up to four used adhesive patches. The four regions of the placement area panel are not exposed to any skin prior to application of a used patch.

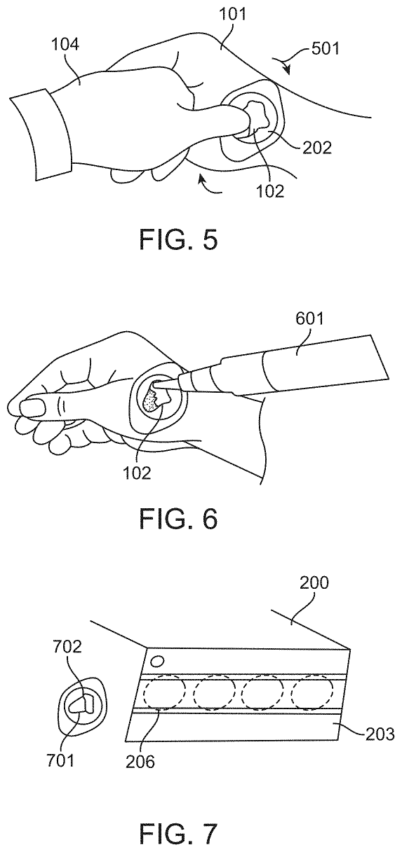

An adhesive patch is removed from the top left side of the peelable release panel as exemplified in FIG. 3. The practitioner (104) handles the adhesive patch (202) by the tab region (301) so that the matrix material of the central collection area (302) does not come in contact with a surface prior to skin application. The skin sampling area is held taut while the adhesive patch is applied onto the skin sampling area. An adhesive patch (202) positioned on the cleansed skin sampling area (101) comprising a skin lesion (102) is exemplified in FIG. 4. The adhesive patch is pressed firmly on the skin while making 15 circular motions. FIG. 5 exemplifies the practitioner (104) pressing on the skin comprising a skin lesion (102) while making a circular motion (501). As exemplified in FIG. 6, the lesion area (102) is demarcated on the adhesive patch (202) using a marker (601) provided in the skin sample collection kit exemplified in Example 2. The practitioner slowly removes the used adhesive patch from the skin sampling area by holding the tab and pulling in one direction. The used patch (701) comprising a skin sample (702) is placed on the first unoccupied skin collection region (206) of the placement area panel (203) on the tri-fold skin sample collector (200) as exemplified in FIG. 7. The procedure is repeated with three additional patches on the same lesion.

The tri-fold skin sample collector is folded and placed in a package provided with the skin sample collection kit. The package contains pre-paid postage and is self-addressed to a processing facility.

### Example 2: Skin Sample Collection

A pigmented lesion located on the upper back of a subject is selected for skin sampling. The skin sampling area contains a minimal amount of hair, is not irritated and has not been previously biopsied. The lesion is about 15 mm in size. The lesion is sampled utilizing an adhesive skin sample collection kit. The skin sample collection kit includes an instructions for use sheet (or an instruction manual). The lesion is sampled by a capable person who has read and understood the skin sample collection kit instructions for use sheet.

A pair of gloves is removed from the skin sample collection kit and the fitted onto the person performing the skin sampling procedure. The skin sampling area comprising the pigmented lesion is cleansed with an alcohol pad provided in the adhesive skin sample collection kit and the skin is allowed to air dry.

A tri-fold skin sample collector is removed from the adhesive skin sample collection kit. The tri-fold skin sample collector comprises a peelable release panel comprising four adhesive patches, a placement area panel comprising a removable liner, and a clear panel. The tri-fold skin sample collector has a barcode specific for the subject. The tri-fold skin sample collector further comprises an area configured for providing patient information. The tri-fold skin sample collector is labeled with the subject's name and identifying information. The removable liner is removed from the placement area panel, exposing four regions designated for the placement of up to four used adhesive patches. The four regions of the placement area panel are not exposed to any skin prior to application of a used patch.

An adhesive patch is removed from the top left side of the peelable release panel. The adhesive patch is handled by the tab region so that the matrix material does not come in contact with a surface prior to skin application. The skin is held taut while the adhesive patch is applied onto the skin sampling area. The adhesive patch is pressed firmly on the skin while making 10 circular motions. The lesion area is demarcated on the adhesive patch using a marker provided in the adhesive skin sample collection kit. The used patch is slowly removed in one direction by pulling the tab away from the skin. The used patch is placed on the first unoccupied skin collection region of the tri-fold skin sample collector. The skin sample procedure is repeated with three additional patches on the same skin lesion.

The tri-fold skin sample collector comprising 4 used adhesive patches is folded and placed in the package provided with the adhesive skin sample collection kit. The package contains pre-paid postage and is self-addressed to a diagnostics facility.

### Example 3: Collection System

The adhesive skin sample collection kit components are stored in a cardboard box (800) as exemplified in FIG. 8. The kit contains a tri-fold skin sample collector (200) comprising four adhesive patches, instructions for use sheet, a marking pen, a pre-paid, self-addressed shipping package (801), and a shipping label (802). The tri-fold skin sample collector comprises three panels including a peelable release panel comprising the four adhesive patches, a placement area panel comprising a removable liner and a clear panel. The tri-fold skin sample collector further comprises a unique barcode (803) configured to identify a subject. The adhesive patches stored on the peelable release panel have an expiry date of 2 years from the date of manufacture. The skin sample collection kit is stored between 10° C. and 30° C. The instructions for use sheet (or instruction manual) include all information necessary to enable a person to understand and perform the method. The instructions for use sheet (or instruction manual) include diagrams describing steps of the skin sample collection method.

### Example 4: Biological Sample Storage with Desiccant

Background Quality of biological samples with living cells may decline when exposed to moisture at room temperature. Previous studies indicated nucleic acids in skin samples collected on adhesive patches are stable at room temperature for at least 10 days if not exposed to moisture or a broken cold-chain (Yao et al., J Drugs Dermatology (2017) October 1; 16(10):979-986). Dried skin tissues with inactive nuclease on patches has allowed transport of patch-collected skin samples from collection sites (clinics) to analytical laboratory at room temperature by overnight Fedex or UPS. This greatly increases the convenience of the sample handling and reduces the cost on sample transportation. However slight changes in nucleic acid yields isolated from these samples was observed in a cyclic (or seasonal) pattern, e.g., a reduction of yield in samples shipped (at room temperature) in summer months (data not shown). As the decrease in nucleic acid yield appears to match to the seasonal changes in air humidity and temperature, summer decline in sample nucleic acid yields may relate to the summer increase in air humidity, which causes moisture to build up (condensation) in these previously dried skin samples on patch and activate the nucleases in the skin samples on patches. This may eventually lead to the breakdown of nucleic acids and the decline of nucleic acid yields in these samples. Without being bound by theory, the accompanying increase of air temperature in summer months might have accelerated this nucleic acid breakdown caused by increased humidity. Procedures were evaluated (e.g., use of desiccant) to maintain the quality of samples (preventing or minimizing nucleic acid loss) in skin samples shipped on patches at room temperature in summer months.

Test Design and Procedure. The procedure included 2 major groups of samples, one in resealable plastic or foil bags with desiccant and one in the same bags without desiccant, and all bags incubated (stored) in a humidity chamber with high air humidity (70%). Following a period of incubation (storage), nucleic acids are isolated from all samples (from bags with or without desiccant) to compare the nucleic acid yields, determined by RT-qPCR on RNA using a human housekeeping gene (beta actin). RNA yields from the 2 groups of samples are compared to determine if and how desiccant helps preserving the nucleic acids in samples stored under this condition.

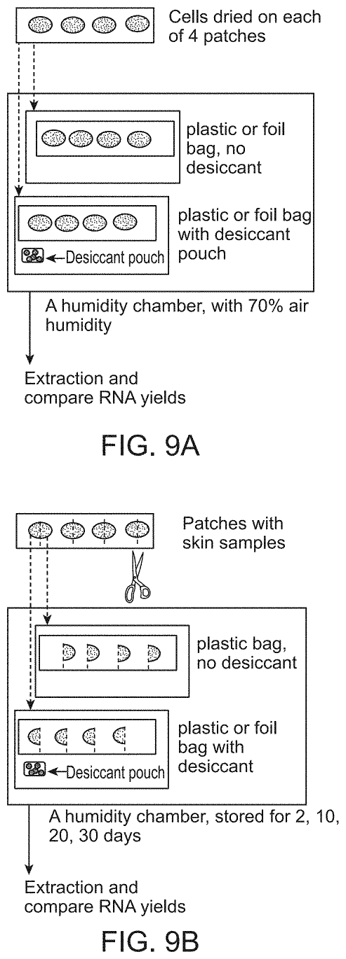

Desiccant effect and sample transportation bag. The initial measurement of desiccant effect was conducted using cells from a cultured cell line to create a comparable equal input of starting material (cells) on each adhesive patch. To do that, 5 uL of a well-mixed cell solution was spot on to the sticky side of each adhesive patch (5 uL per patch), a large number of the cell-spot patches were prepared, and cell solution allowed to dry overnight on each patch. With these dried cell-loaded patches, following test was carried out: Group1: 8 patches stored in −80° C. freezer for 2 days as control (‘T0 Frozen’); Group2: 8 patches placed in a resealable plastic bag (without desiccant); Group3: 8 patches placed in a resealable plastic bag with 1 desiccant pouch (0.5 gram silica gel desiccant); Group4: 8 patches placed in a resealable plastic bag with 4 desiccant pouches (2 g); and Group5: 8 patches placed in a resealable plastic bag with 10 desiccant pouches (5 g). All resealable bags from Groups 2-5 were then stored in an enclosed plastic box with 70% of air humidity (a humidity chamber). Placed the humidity chamber in a cardboard box and left the box at outdoor in the balcony outside the R&D lab (mimicking a sample transportation condition by Fedex or UPS). (FIG. 9A). After 2 full days (48 hours) incubation in this humidity chamber, all resealable bags were removed from the box and nucleic acids extracted from all patches (one patch per extraction), from bags with or without desiccant. Samples stored at −80° C. freezer (from Group1) were also extracted together with the samples from other groups. To examine the effect of sample transportation bags on desiccant effect, a parallel test with the same test design to the above resealable bag was conducted using foil bags. To measure the time course effect of desiccant on preserving the samples on patch, the dried cell-loaded patches were placed in bags without desiccant (control) and with 4 desiccant pouches (2 gram silica gel desiccant) and left in the humidity chamber for 2, 10 and 20 days, respectively, before proceeding to sample extraction and nucleic acid yield comparison.

More tests were conducted to validate the desiccant effect observed from the above dried cells tests in actual skin samples collected on adhesive patches. As the biomass of skim sample collected on each patch varies greatly from one patch to the other, it is in some instances difficult to create an equal input of skin samples for the desiccant test between groups. Instead of comparing samples between patches, a test was designed to skin samples from the same patch. Each sample-loaded patch was cut in half, one half placed in bags without desiccant (control) and one half placed in bags with desiccant (test), as shown in FIG. 9B. This creates an equal (or mostly equal) biomass of skin samples on patches for the desiccant test. These bags were incubated in the humidity chamber for 2, 10, 20 and 30 days, respectively, to allow examination of desiccant effect on real skin samples in patch as well as the time course effect of desiccant on nucleic acids in skin samples on patches stored in humid environment. Nucleic acid extraction from dried cells or skin samples on adhesive patches and quantification of the isolated nucleic acids from these samples followed standard operation procedures.

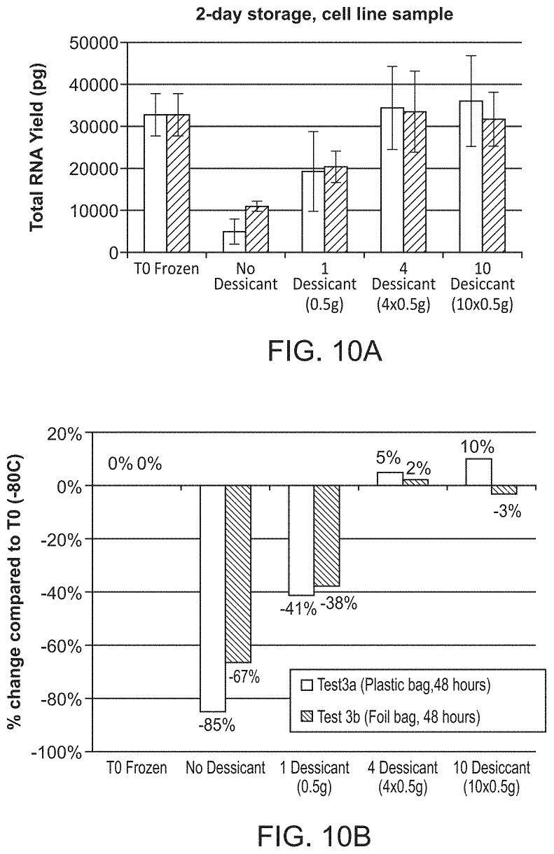

Desiccant effect on samples exposed to high air humidity. FIG. 10A depicts total RNA yields isolated from the dried cells on adhesive patches stored for 2 days in different conditions, including that stored at −80° C. (To Frozen), in humidity chamber without desiccant, and with 1, 4, and 10 desiccant pouches, and from patches stored in resealable plastic bags (no hatching) or in foil bags (hatched bars), all after 2 day (48 hours) storage. Bar heights represent the averaged total RNA yield per patch calculated from 8 repeats with standard deviation from each test condition. As the storage at −80° C. freezer is the standard storage method for most biological samples, RNA yields from this group (To Frozen) demonstrate the total RNA yield (>30,000 pg) that was present in samples on each patch. Samples from patches exposed to a humid environment for the same period of time (2 days) without desiccant protection (No desiccant) yielded a significantly less amount of total RNA, an average of than 65% less of total RNA (67% to 85%) compared to that from Group1 (stored in −80° C. freezer, FIG. 10B) These data confirm the impact of a high air humidity environment on the yield (and quality, reflected through the yield loss) of nucleic acids in samples stored on patches at room temperature. Desiccant appears to have counter acted the high air humidity and helped protect the nucleic acids in the samples stored in the same humid environment as including just 1 desiccant pouch (0.5 g) in the storage bags had enabled us to recover about 20,000 pg of total RNA, which is twice of that from the bags without desiccant, or to reduce RNA loss by ˜50% (cutting RNA loss from 85% to 40%, FIG. 10B). The desiccant effect appears to be dose dependent (FIG. 10A and FIG. 10B), and with 4 desiccant pouches in the storage bags, the desiccant was able to significantly reduce or eliminate the RNA yield loss in samples caused by a humid environment (compared to that stored at −80° C.).

This result clearly demonstrates the effect of desiccant on preserving the nucleic acids in samples stored in humid environment, likely through absorbing moisture from the air around the samples to keep the samples on patches dry, which prevents the nuclease activation and nucleic acids degradation in the samples. A storage condition with 4 desiccant pouches (2 g silica gel desiccant) in the bags appears to be able to remove all or nearly moisture from the air and preserve (maintain) the samples in good quality. This test was repeated 3 times, and all showed a similar trend of desiccant effect, confirming the observations shown in this report (FIG. 10A and FIG. 10B). In addition, resealable plastic bag or foil bag have worked similarly or equally well. Moving forward, we adopted a condition with a combination of foil bag and 2 grams of desiccant (4 desiccant pouches) for storage of samples at room temperature for the remaining tests.

Time course of desiccant effect. FIG. 11 shows the percentage (fold) of RNA yield change from samples stored in foil bags with 4 desiccant pouches (dotted line) and without desiccant pouch (solid line, control) in a humid chamber (70% humidity) for 2, 10 and 20 days, compared to the RNA yields from samples extracted fresh (on day 0). Without desiccant protection, RNA in samples lost quickly (total yield reduced 67% by day 2 and nearly 100% after 10 days). In contrast, with 4 desiccant pouches in the storage bags, no or minimal RNA loss was seen in the first 2 days and only ˜7% of loss incurred in the first 10 days. These data again demonstrate that desiccant maintains the quality of samples on patches and protects the nucleic acids in samples exposed to humid environment from degradation. With a 6×8″ bag, 4 desiccant pouches (2 g) may remain effective for up to 10 days. Desiccant in these bags may eventually be saturated by the moisture with extended time and lose protection on samples (RNA yield dropped 64% by day 20 in bags with 4 desiccant pouches).

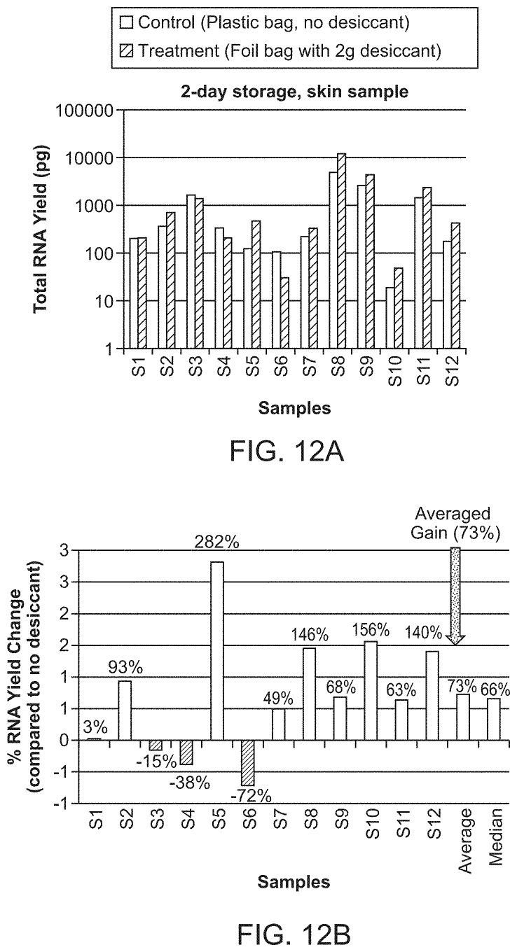

Validation of desiccant effect on real skin samples on patches. FIG. 12A shows total RNA yields isolated from skin patches collected from the skin of 12 subjects (human volunteers). Each skim patch was cut in half, one half stored in bags without desiccant and one half stored in bags with 4 desiccant pouches, and all bags stored in a humidity chamber (70% humidity) for 2 days before RNA extraction (FIG. 9B).

FIG. 12B shows % of RNA yield change between the 2 conditions (stored with and without desiccant), calculated as %=(RNA Yield-with desiccant−RNA Yield Without desiccant)/RNA Yield without desiccant, for each subject. Of these 12 subjects, 9 demonstrated increased RNA yield from patches stored with desiccant in humid chamber, ranging from 5% to >280% gain with an average gain of 73% (or a median gain of 66%). This test confirms the desiccant works on the actual skin samples similar to the effect on dried cells on patches stored in humid environment.

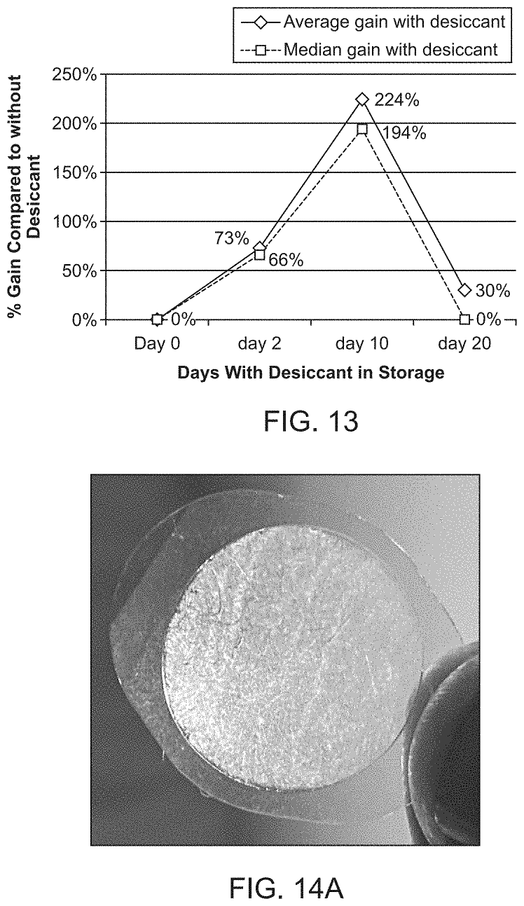

A time course study to evaluate desiccant humid environment was also conducted. FIG. 13 shows that the % of RNA yield gain from patches stored in foil bags with 4 desiccant pouches (per bag), compared to their counterpart stored in bags without desiccant, in humidity chamber for 2, 10 and 20 days. The % of RNA yield gain is calculated with the same formula shown above (FIG. 12B), with solid line for the averaged gain (of the 12 subjects) and dotted line for the median gains of the same sample sets. Both calculations (average or median) give the same trend of RNA yield gain change, or ˜70% gain by day 2 and ˜200% gain by day 10 from samples stored with desiccant. The high gain rate seen on day 10 (>200%) was likely due to a significant loss of (or lower) RNA yields in the group stored without desiccant, causing a much smaller denominator in the above % gain calculation equation. By day 20, the RNA gain from the group of skin samples stored with desiccant has returned to ˜0%, likely as a result of the desiccant being saturated by moisture and unable to protect the nucleic acid in samples, consistent with what was seen from the dried cells test (FIG. 11).

Summary. This study confirms that a high air humidity during room temperature storage of samples (dried cells or skin) on patch may cause RNA yield to decrease. Including desiccant to the sample storage bags may help reduce or prevent the negative impact of air humidity on samples during the room temperature storage. Storing the patch-collected skin samples in 6×8″ foil bag with 2 grams of silica gel desiccant (4 desiccant pouches, 0.5 g each, per bag) may help eliminate the negative impact of high air humidity and maintain the RNA yields unchanged at room temperature for up to 10 days. A new sample storage procedure has been developed based on this study and transferred to clinical sample transportation test in CLIA laboratory.

### Example 5: Properties and Design of Patches

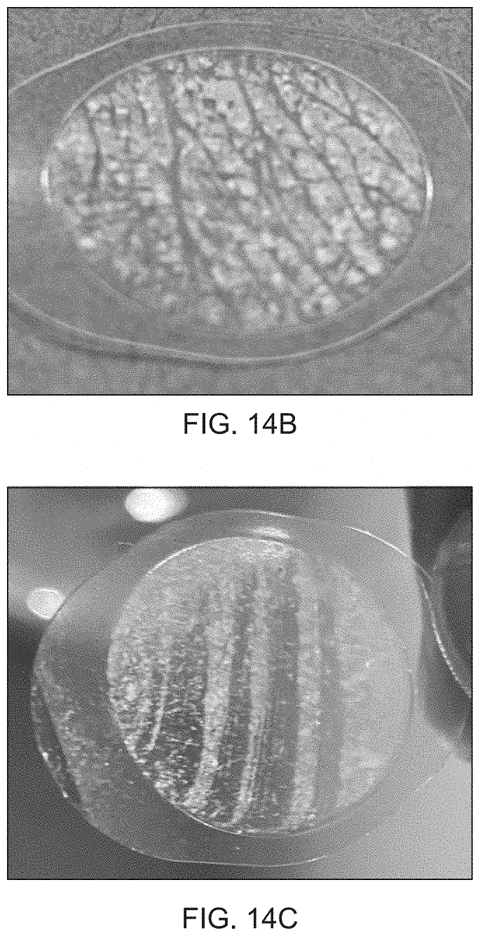

Various properties of patches used in Examples 1-3 were measured. “Wrinkling” (depicted in FIG. 14B and FIG. 14C), without being bound by theory, is related to the combination of backing thickness+modulus of elasticity+overall dimensions of the patch. In general, resistance to wrinkling and folding should increase exponentially as the thickness of the backing increases (assuming the same modulus and dimensions). For comparison, a patch without wrinkling (depicted in FIG. 14A) may result in higher collection. Wrinkling is also believed to be related to the amount of tension on the skin during application of the patch. The issue of the patch folding over or sticking to itself during handling is related to these same properties, so improvements in one should result in improvements in the other. “Skipping” (also sticking and slipping herein) also creates distortions, but they are more microscopic in nature than those distortions caused by the wrinkling effect. In some instances, skipping may impact or correlation to performance. In some instances, skipping is reduced with controlled peel methodology, e.g., by peeling at a 90 degree angle or even greater (folding the patch backing on itself during peeling up to an angle of 180 degrees from where it started on the skin).

Glass transition temperature is another physical property of the adhesive polymer structure (regardless of chemistry) related to how the polymer chains interact with each other and certain properties resulting therefrom (e.g., viscosity). Tensile Strength is in some instances measured using the same ASTM standard as modulus of elasticity.

Patches for use in the general procedures of Examples 1-3 are designed according to the parameters of Table 1.

TABLE 1

| Adhesive | | | Anticipated |
|---|---|---|---|
| Name | Thickness | Composition | Strength |
| H-72 | 1.0 mil | Rubber | ~10 N/in |
| H-72 | 2.0 mil | Rubber | ~13 N/in |
| H-52 | 0.5 mil | Rubber | ~16 N/in |
| H-52 | 1.0 mil | Rubber | ~20 N/in |
| ARcare 90068 | 2.0 mil | Rubber | 17.5 N/in |
| 160-49 | 0.5 mil | Acrylic | ~10 N/in |
| 160-49 | 1.0 mil | Acrylic | ~13 N/in |
| 160-49 | 1.5 mil | Acrylic | ~16 N/in |
| 160-49 | 2.0 mil | Acrylic | ~20 N/in |
| 160-49 | thicker than 2.0 mil | Acrylic | more than 20 N/in |
| ARcare 90068 | 2.0 mil | Rubber | 17.5 N/in |

1 mil = 0.001 inches.

### Example 6: Water-Soluble Patches and/or Adhesives

Patches of Examples 1-3 are designed with modifications of patch properties and adhesives: combinations of (a) water soluble patches with non-water soluble adhesives, (b) water soluble patches with water soluble adhesives, or (c) non-water soluble patches with water soluble adhesives.

Water soluble adhesive patches with water-soluble adhesive are designed to provide >12 oz/in² loop tackiness and a working temperature range from −40 to 176° F. on a water-soluble paper backing that gives >20 lb/inch tensile force and >200 mN tear strength. The entire soluble patch is dissolvable in any water temperature easily and quickly (within 30 seconds), leaving no adhesive residue, and has an expected shelf life of 12 months. The water soluble adhesive patch is used for non-invasive skin sample collection. Collected (skin sample-loaded) patches are subjected to lysis incubation for nucleic acid extraction following the general procedures of Examples 1-3. As the water soluble patch will dissolve during lysis incubation, there is no need to remove them during and after the sample lysis incubation.

### Example 7: Hybrid Adhesive Patches

Patches of Examples 1-3 are designed with modification: the adhesive is replaced with a hybrid adhesive comprising one or more components, and the backing layer is modified as shown in Table 2. Various thicknesses (0.5-3.0 mil) patches are tested with the skin sampling methods described herein.

TABLE 2

| | | | Adhesive | Adhesive |
|---|---|---|---|---|
| | Thick- | Backing | component | component |
| Number | ness | Layer | 1 | 2 |
| 1 | 0.5 mil | polyethylene | Rubber | Silicone |
| 2 | 1.0 mil | polyethylene | Rubber | Silicone |
| 3 | 1.5 mil | polyethylene | Rubber | Silicone |
| 4 | 2.0 mil | polyethylene | Rubber | Silicone |
| 5 | 3.0 mil | polyethylene | Rubber | Silicone |
| 6 | 0.5 mil | polyethylene | Rubber | Acrylic |
| 7 | 1.0 mil | polyethylene | Rubber | Acrylic |
| 8 | 1.5 mil | polyethylene | Rubber | Acrylic |
| 9 | 2.0 mil | polyethylene | Rubber | Acrylic |
| 10 | 3.0 mil | polyethylene | Rubber | Acrylic |
| 11 | 0.5 mil | polyester | Rubber | Silicone |
| 12 | 1.0 mil | polyester | Rubber | Silicone |
| 13 | 1.5 mil | polyester | Rubber | Silicone |
| 14 | 2.0 mil | polyester | Rubber | Silicone |
| 15 | 3.0 mil | polyester | Rubber | Silicone |
| 16 | 0.5 mil | polyester | Rubber | Acrylic |
| 17 | 1.0 mil | polyester | Rubber | Acrylic |
| 18 | 1.5 mil | polyester | Rubber | Acrylic |
| 19 | 2.0 mil | polyester | Rubber | Acrylic |
| 20 | 3.0 mil | polyester | Rubber | Acrylic |
| 21 | 0.5 mil | polyethylene | Silicone | Acrylic |
| 22 | 1.0 mil | polyethylene | Silicone | Acrylic |
| 23 | 1.5 mil | polyethylene | Silicone | Acrylic |
| 24 | 2.0 mil | polyethylene | Silicone | Acrylic |
| 25 | 3.0 mil | polyethylene | Silicone | Acrylic |
| 26 | 0.5 mil | polyester | Silicone | Acrylic |
| 27 | 1.0 mil | polyester | Silicone | Acrylic |
| 28 | 1.5 mil | polyester | Silicone | Acrylic |
| 29 | 2.0 mil | polyester | Silicone | Acrylic |
| 30 | 3.0 mil | polyester | Silicone | Acrylic |

1 mil = 0.001 inches.

### Example 8: Comparative Study

The following example describes results from a study designed to compare the performances of skin sample collectors, such as those described in commonly owned WIPO Publication No. WO2016/179043, which is incorporated by reference herein in its entirety, with thirteen different collections systems (e.g., tapes) in sampling epidermal tissue and yielding extracts of epidermal nucleic acids suitable for biomarker analyses. Twelve of the thirteen tapes comprise embodiments, variations, or examples of the skin sample collectors and collection systems described herein. The D-Squame skin sampling disc (CuDerm Corp) was also used as a comparator device. An improved tape would consistently improve the yields and quality of nucleic acids, while reducing skin injury to maintain non-invasive properties.

Background—Use of adhesive tapes for the collection of stratum corneum (tape stripping) is a versatile and minimally invasive procedure applied in several different fields, including assessments of the skin barrier function, microbial content, and disease biomarkers. Tape stripping is referenced in various published studies; however, the number of tapes vary widely, and the lack of standardized sampling and normalization protocols complicate data comparison and interpretation. Moreover, tapes differ in their formulations, which may be relevant for several aspects of their performance, including the extent of tissue sensitization, yields of nucleic acids and downstream assay applications. There is a need for improved sampling tapes, which would enable non-invasive epidermal tissue collection while consistently obtaining high nucleic acid yield and quality to support the identification of translatable epidermal biomarkers.

Further, Skin sampling with sample collections systems may result in a skip pattern on the adhesive surface and a “jerky” feel of the peel. The skip pattern may be related to the energetic instability of the peel front resulting from a combination of the mechanical properties of the sticker and the skin substrate; this may decrease the sampling comfort and efficiency.

Summary—Thirteen different collection systems were profiled. Twelve of the thirteen tapes comprise embodiments, variations, or examples of the skin sample collectors and collection systems described herein. D-Squame skin sampling disc (CuDerm Corp, “DSQ” herein) was also used as a comparator device. A skin sample collector, such an example, variation, or embodiment as those described in commonly owned International Patent Publication No. WO2016/179043, which is incorporated by reference herein in its entirety, was also used as a comparator device (“CC” herein). Composition and thickness of the adhesives and the backing layers were determined for each tape via Fourier Transform Infrared Spectroscopy (FTIR) and any leachables/extractables (volatile residuals, additives, fillers, binders, etc.) were analyzed and identified by GC-MS extraction and gravimetric analysis using Ethanol and Isopropanol (which are commonly present in buffers used for DNA and RNA isolation). Peel adhesion force of the tapes was determined by the ASTM D3330 180° peel adhesion standard method using XLW (EC) Auto Tensile Tester (Labthink Instrument, Inc.).

It was shown that increasing the thickness of the backing sheets is likely to lower the probability of the skip patterns forming on the adhesive surface while sampling.

### Materials and Methods

Twelve example collection systems of the present disclosure were selected for study. Each collection system comprised the adhesive 160-49 (medical grade 2-ethylhexyl acrylate polymer) and EVA (22% Vinyl acetate, 78% Ethylene) backing sheet, manufactured by the Lamart Corp. and Wiman Corp., respectively. The twelve were further customized to have different thicknesses, but otherwise identical formulations of the adhesives and the backing sheets. A summary of the example devices is as shown in Table 3:

TABLE 3
Tape types/Sample Summary

| | Tape | Adhesive | Thickness (mil) | |
|---|---|---|---|---|
| Sample | Backing ID | ID | Adhesive | Backing |
| T1 | .004 × 12″ | Lamart | 1 | 4 |
| | PEO3000 Clear | 160-49 | | |
| | Matte mPE film | | | |
| T2 | .005 × 12″ | Lamart | 1 | 5 |
| | PEO3000 Clear | 160-49 | | |
| | Matte mPE film | | | |
| T3 | .006 × 12″ | Lamart | 1 | 6 |
| | PEO3000 Clear | 160-49 | | |
| | Matte mPE film | | | |
| T4 | .004 × 12″ | Lamart | 1.5 | 4 |
| | PEO3000 Clear | 160-49 | | |
| | Matte mPE film | | | |
| T5 | .005 × 12″ | Lamart | 1.5 | 5 |
| | PEO3000 Clear | 160-49 | | |
| | Matte mPE film | | | |
| T6 | .006 × 12″ | Lamart | 1.5 | 6 |
| | PEO3000 Clear | 160-49 | | |
| | Matte mPE film | | | |
| T7 | .004 × 12″ | Lamart | 2 | 4 |
| | PEO3000 Clear | 160-49 | | |
| | Matte mPE film | | | |
| T8 | .005 × 12″ | Lamart | 2 | 5 |
| | PEO3000 Clear | 160-49 | | |
| | Matte mPE film | | | |
| T9 | .006 × 12″ | Lamart | 2 | 6 |
| | PEO3000 Clear | 160-49 | | |
| | Matte mPE film | | | |
| T10 | .004 × 12″ | Lamart | 2.5 | 4 |
| | PEO3000 Clear | 160-49 | | |
| | Matte mPE film | | | |
| T11 | .005 × 12″ | Lamart | 2.5 | 5 |
| | PEO3000 Clear | 160-49 | | |
| | Matte mPE film | | | |
| T12 | .006 × 12″ | Lamart | 2.5 | 6 |
| | PEO3000 Clear | 160-49 | | |
| | Matte mPE film | | | |

The comparator skin sample collector example (CC), such as described in commonly owned International Patent Publication No. WO2016/179043, which is incorporated by reference in its entirety, has the following properties: Medical grade MA-70 adhesive thickness 3 mil, polyurethane backing thickness 3 mil, and adhesive peel force 18.1 N/25 cm.

As described further herein, tests of the mechanical properties of the twelve tapes were performed using instrumental techniques such as ASTM D3121 tack and ASTM D3330 180° peel adhesion tests, as well as testing for comfort and wrinkles on skin (see also e.g., Example 10 herein). A summary of results is presented in Table 4.

TABLE 4
Summary of tape properties tested

| | | | | Tack | | |
|---|---|---|---|---|---|---|
| | | | | (avg. | | |
| | | | | Distance | | |
| | Thickness | | Peel | ball | | Dis- |
| Tape | Adhesive | Backing | force | travelled | Avg. | comfort |
| ID | (mil) | (mil) | (N/25 cm) | (cm)) | Wrinkles | (1-5) |
| T1 | 1 | 4 | 7.66 | 8.6 | 9 | 1 |
| T2 | 1 | 5 | 10.67 | 7.3 | 2.3 | 1.7 |
| T3 | 1 | 6 | 10.42 | 7.1 | 2.3 | 1.3 |
| T4 | 1.5 | 4 | 15.9 | 4.4 | 5 | 2 |
| T5 | 1.5 | 5 | 15.95 | 4.2 | 4 | 2.3 |
| T6 | 1.5 | 6 | 17.75 | 4.1 | 3.3 | 2.3 |
| T7 | 2 | 4 | 15.62 | 3.8 | 6.9 | 3.3 |
| T8 | 2 | 5 | 17.79 | 3.7 | 7 | 3.3 |
| T9 | 2 | 6 | 18.58 | 3.7 | 4.6 | 3 |
| T10 | 2.5 | 4 | 17.21 | 2 | 7 | 2.7 |
| T11 | 2.5 | 5 | 18.58 | 1.6 | 5.7 | 3.3 |
| T12 | 2.5 | 6 | 20.33 | 1.4 | 4.7 | 3.7 |

Recruitment of Subjects—A total of twenty-one healthy volunteers were recruited for the study (71% female). All subjects were employees of Applicant that have consented to the use of their samples in the study. Subject IDs and gender are listed in the Table 5. Two R&D scientists performed the sampling, each covering approximately 50% of the subjects. The scientists were trained on the sampling procedure for two days prior to the study.

TABLE 5
Subjects recruited for the study

| Subject | Subject | |
|---|---|---|
| Number | ID | Gender |
| Subject 1 | 01-211 | F |
| Subject 2 | 01-210 | F |
| Subject 3 | 01-214 | F |
| Subject 4 | 01-213 | F |
| Subject 5 | 01-215 | M |
| Subject 6 | 01-174 | F |
| Subject 7 | 01-212 | F |
| Subject 8 | 01-216 | M |
| Subject 9 | 01-217 | F |
| Subject 10 | 01-219 | M |
| Subject 11 | 01-051 | F |
| Subject 12 | 01-105 | M |
| Subject 13 | 01-177 | M |
| Subject 14 | 01-218 | F |
| Subject 15 | 01-221 | F |
| Subject 16 | 01-079 | M |
| Subject 17 | 01-088 | F |
| Subject 18 | 01-220 | F |
| Subject 19 | 01-153 | F |
| Subject 20 | 01-173 | F |
| Subject 21 | 01-181 | F |

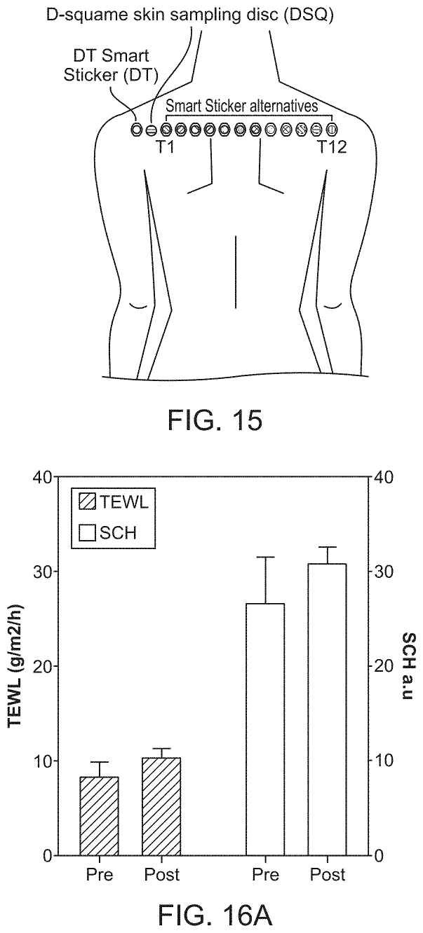

Study Design Summary—A side-by-side comparison was performed by sampling the same 20 healthy volunteers on adjacent non-overlapping sites on the upper back with four tapes per prototype. FIG. 15 illustrates example positions of 14 sampling tapes on selected upper back sites. Prior to sampling, all subjects were required to review and understand the study scope and formally consent to having their skin sampled. The exact position of the different tapes along the upper back was randomized for each subject. The location of the first tape in each set was marked with skin-safe pen, to guide placement of subsequent tapes and ensure stripping from the same spot. The D-squame pressure instrument was applied to all tapes for 5 seconds before stripping. All samples collected were frozen at −80° C. until the extraction of nucleic acids that was started the day following the sampling.

Performance of the tape prototypes was determined by evaluating three main testing categories: skin barrier function, subject discomfort, and quantity and quality of extracted nucleic acids. A point-scoring system was assigned to every sub-category to facilitate direct comparison. Sum of points from each test yielded a final score for every tape, which was used to rank the tapes from the most to the least performing in this study cohort.

### Results

Results from the following testing categories are presented, each consisting of several sub-categories:

### 1. Skin Barrier Function

i. Trans epidermal water loss (TEWL)
ii. Hydration of stratum corneum (SCH)

### 2. Subject Discomfort

i. Self-reporting of discomfort by subjects during sampling
ii. Visually observable erythema post-stripping
   3. Yields and quality of nucleic acids (DNA/RNA)
i. Total yields
ii. RNA integrity and fragment size distribution
iii. Consistency of yields across subjects
iv. Percentage of samples of sufficient quantity to run melanoma assay
   4. Scores considering the above sub-categories

### 1. Skin Barrier Function

Background—The epidermis provides a barrier between the organism and the outside environment by protecting against physical and chemical insults, preventing microbial infection, and limiting passive water loss. In skin, deeper dermal layers are highly moisturized and there is a passive water diffusion gradient from the deeper dermal layer toward the stratum corneum; most of the diffused water is evaporated from the skin surface, however a fraction is retained by the protein-rich corneocyte within the stratum corneum. Adequate hydration of the stratum corneum is important for the maintenance of chemical and mechanical properties of the epidermis and intact stratum corneum directly regulates these properties by determining the amounts of retained and lost water. These two properties, the extent of passive water loss (transepidermal water loss—TEWL) and the moisture content in the stratum corneum (stratum corneum hydration—SCH) are commonly used as proxies for determining the skin barrier function.

Without being limited by theory, higher TEWL is associated with barrier impairments while lower TEWL is indicative of intact barrier function; conversely, higher steady state SCH indicates a healthy barrier, while lower SCH levels may suggest barrier impairments, especially when paired with an increased TEWL. Assuming sampling in ambient temperature (22-26° C.) and 45-60% humidity, baseline TEWL and SCH values for intact barrier mostly vary depending on the body site. According to the literature, healthy median TEWL at dry body sites is expected to measure less than 12-15 g/m2/h and healthy SCH is expected to be between 20-40 (measured in arbitrary units), while disrupted barrier shows TEWL higher than 30 g/m2/h7-12. Immediately after tape stripping, both TEWL and SCH are expected to temporarily increase due to perturbations of the outermost layers and the consequent drawing of water toward the stratum corneum. Significant barrier disruption would eventually lead to lower levels of SCH as the skin reaches the new steady state. The difference between pre- and post-stripping values can be used as one of the indicators in assessing the extent of skin barrier disruption by repeated tape-stripping.

Methods—Skin barrier function was assessed by measuring the trans epidermal water loss (TEWL) and hydration of the stratum corneum (SCH) with the gpskin Barrier Light device and associated software, before and after tape stripping. To ensure that the measurements were not influenced by perspiration, the subjects were sampled at their workstations and asked to limit physical activity for 30 minutes prior to sampling. These measurements informed on the skin barrier function, before and after tape stripping.

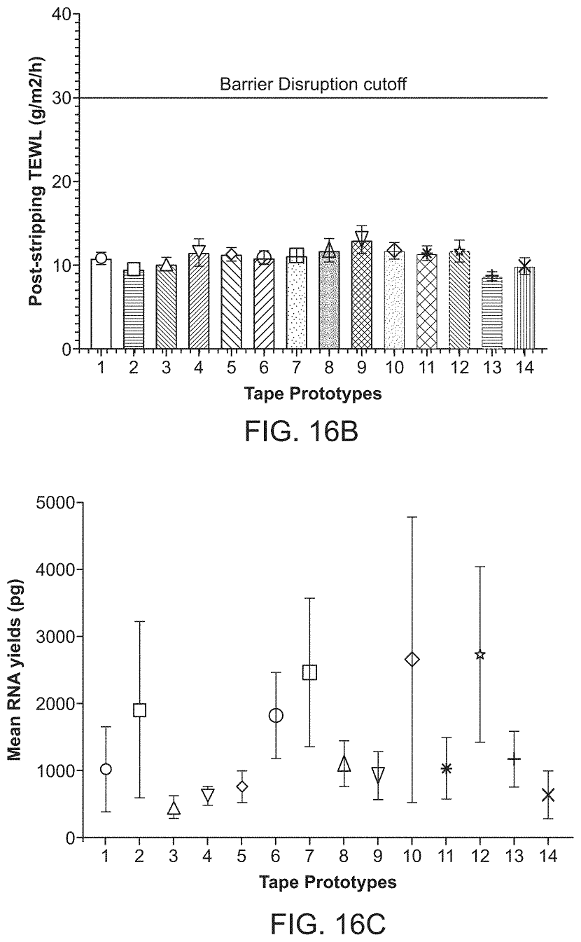

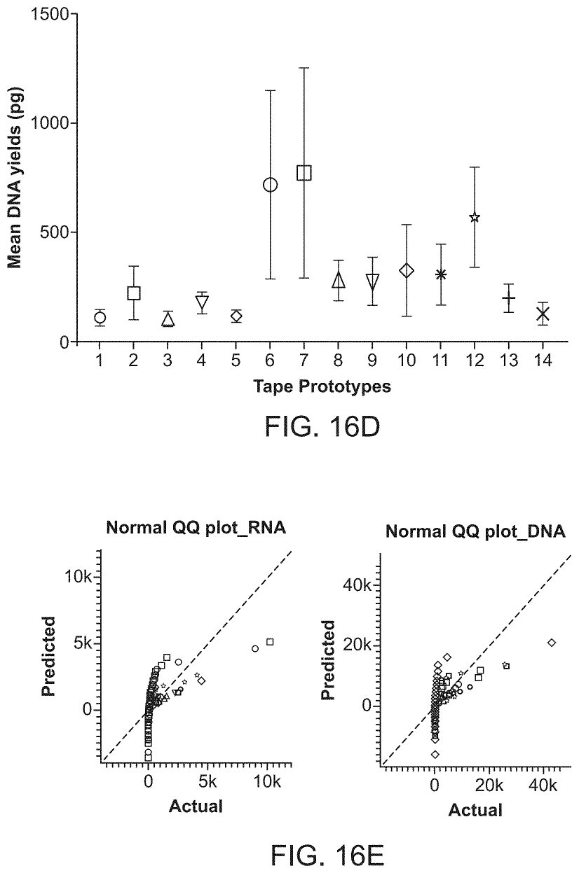

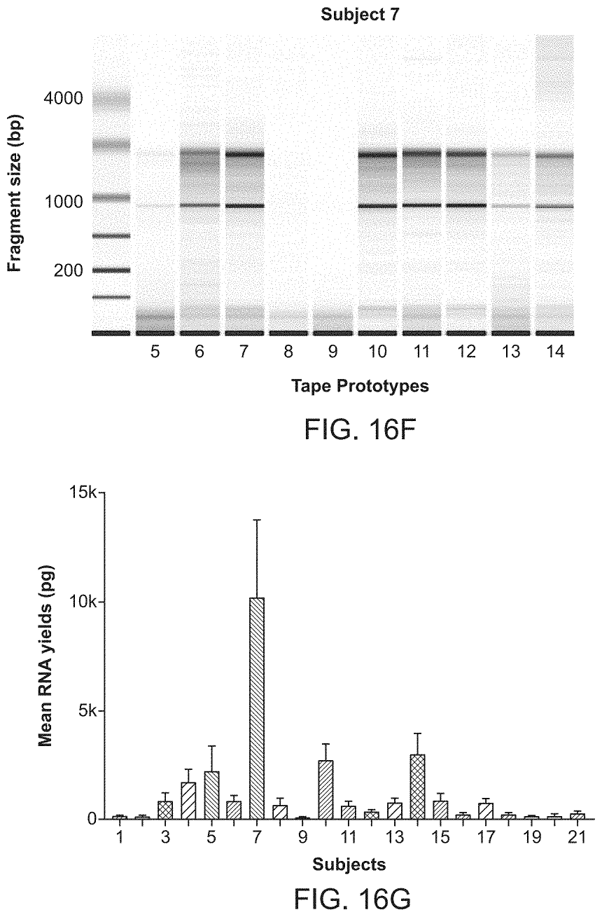

Results—FIG. 16A illustrates a graph of levels of TEWL and SCH before and after stripping with four consecutive tapes. Bar graphs represent average values for all tapes (N=14). FIG. 16B illustrates post-stripping TEWL values for individual tapes (N=21). Barrier disruption cutoff of 30 g/m²/h is indicated with a horizontal line. In the study cohort, median TEWL and SCH values were 9.35±1.8 g/m²/h and 22±1.94 au, respectively, consistent with previously published data for healthy skin (FIG. 16A). After tape-stripping, the levels of TEWL and SCH increased only modestly, to 11.2±1.03 g/m²/h and 31±1.6 au, respectively (FIG. 16A). Since TEWL values recorded post-stripping were below the 30 g/m²/h cutoff for damaged barrier for all tape prototypes, no points were assigned to this sub-category (FIG. 16B).

### 2. Subject Discomfort and Erythema

Methods—During tape stripping, the subjects were asked to rank their level of discomfort with each tape from 1 to 5, with 1 being equivalent to pulling off the softest band-aid and 5 being equivalent to pulling off a very sticky band-aid. Concurrently, the R&D scientists noted any wrinkling or skip patterns on tapes during the sampling.

Results—All subjects provided a discomfort score for each tape immediately after stripping on a scale from 1 to 5, with 1 being equivalent to pulling off the softest band-aid and 5 being equivalent to pulling off a very sticky band-aid. In this cohort, all tapes had a discomfort score equal or lower than 2 (Table 6). Given the general tolerability of the tapes by all subjects, no points were assigned to this sub-category. The R&D scientists performing the sampling recorded the formation of skip patterns or wrinkling on tapes during the procedure. No wrinkles were observed on any of the tape prototypes.

By contrast, in the internal comparator skin sample collector example, skip patterns were present (all subjects, starting from tape number three). R&D scientists performing the sampling procedure provided a binary assessment (present/absent) for the presence of visible erythema within 5 minutes post-stripping with each tape. The median number of subjects with erythema per tape was 2 (range 0-5). Points were assigned to each tape as follows: if the number of subjects with erythema for a given tape was below the median value of 2, the tape was assigned 2 points. Median values were given 1 point and all other values carried zero points (Table 6).

TABLE 6
Erythema points assigned per tape

| | N of | |
|---|---|---|
| | subjects | |
| Tape | with | |
| prototype | erythema | Points |
| T1 | 2 | 1 |
| T2 | 2 | 1 |
| T3 | 0 | 2 |
| T4 | 1 | 2 |
| T5 | 2 | 1 |
| T6 | 2 | 1 |
| T7 | 3 | 0 |
| T8 | 3 | 0 |
| T9 | 5 | 0 |
| T10 | 3 | 0 |
| T11 | 2 | 1 |
| T12 | 4 | 0 |
| T13 (CC) | 0 | 2 |
| T14 (DSQ) | 0 | 2 |

### 3. Quantity and Quality of Nucleic Acids

Methods—Samples were lysed in a modified Norgen lysis buffer (Thorold, ON, Canada) and nucleic acids from the sample lysates extracted with a magnetic bead-based extraction procedure automated on KingFisher Flex (ThermoFisher Scientific, Waltham, Mass.). Extracted nucleic acids were quantified separately by target-specific qPCR analysis.

The quantity of total human RNA was determined by RT-qPCR with a gene expression analysis assay that uses human (3-actin (ACTB) mRNA as a quantified marker. ACTB is a housekeeping gene found in the human genome and demonstrates a stable expression of mRNA product in all cells. This assay contains a detection probe that spans across exons, thus detecting just the expression product (mRNA) of ACTB and not human gDNA. RNA quality was determined after capillary electrophoresis and detection of RNA fragments on Bioanalyzer2100 instrument (Agilent Technologies, Santa Clara, Calif.). The quantity of human genomic DNA (gDNA) was determined by qPCR with a human gene copy number analysis assay (Hs03023880_g1) that uses human ACTB gene in gDNA as a marker.

i. Total Yields

FIG. 16C illustrates a graph of average values for total RNA yields (pg) per tape (N=21). FIG. 16D illustrates a graph of average values for total DNA yields (pg) per tape (N=21). FIG. 16E illustrates QQ plots showing the distribution of RNA (left) and DNA (right) yield values in the 21 subject cohort, compared to a normally distributed population (dotted diagonal line).

Total DNA and RNA were extracted and quantified by qPCR, as detailed in the study design section (FIG. 16C and FIG. 16D), with both average and median yields considered for point assignment. Normal distribution of the RNA and DNA quantities in this subject cohort was assessed by the Shapiro-Wilks test, as well as visually, in a QQ plot (FIG. 16E). It was determined that the quantities of extracted nucleic acids were not normally distributed, therefore more weight was given to median yields as a better indicator of central tendency in skewed datasets. Average yields were determined for each tape and then the median value was determined in that dataset (RNA: median value was 1018.1 pg, range 438-2601 pg; DNA: median value was 251 pg, range 106-774 pg). One point was given to tapes that presented an average value greater than 1018.1 pg. For median yield values, 50% and 75% cutoffs were determined within the whole group (259 pg and 361.5 pg, respectively, for RNA; 86.8 pg and 107.2 pg, respectively, for DNA). Tapes received 2 points if their median yield was above the 75% cutoff and 1 point if it was above 50% cutoff. The tape with the highest value received an additional point. All points were cumulative. Table 7 and Table 8 show quantity point assignment for RNA and DNA, respectively.

TABLE 7
RNA quantity points assigned per tape

| | | Average | | | | | Median |
|---|---|---|---|---|---|---|---|
| Tape | Average | yield | Median | Top | Above | Above | yield |
| prototype | yield | points | yield | score | 75% | 50% | points |
| T1 | 970.64 | 0 | 224.00 | 0 | 0 | 0 | 0 |
| T2 | 1813.62 | 1 | 264.25 | 0 | 0 | 1 | 1 |
| T3 | 437.78 | 0 | 188.07 | 0 | 0 | 0 | 0 |
| T4 | 597.08 | 0 | 361.08 | 0 | 0 | 1 | 1 |
| T5 | 726.84 | 0 | 369.25 | 0 | 2 | 1 | 3 |
| T6 | 1732.84 | 1 | 254.33 | 0 | 0 | 0 | 0 |
| T7 | 2339.48 | 1 | 232.75 | 0 | 0 | 0 | 0 |
| T8 | 1056.20 | 1 | 205.98 | 0 | 0 | 0 | 0 |
| T9 | 878.08 | 0 | 404.83 | 0 | 2 | 1 | 3 |
| T10 | 2529.99 | 1 | 142.28 | 0 | 0 | 0 | 0 |
| T11 | 980.15 | 0 | 361.67 | 0 | 2 | 1 | 3 |
| T12 | 2600.63 | 1 | 470.75 | 1 | 2 | 1 | 4 |
| T13 | 1116.49 | 1 | 280.58 | 0 | 0 | 1 | 1 |
| (CC) | | | | | | | |
| T14 | 610.55 | 0 | 110.83 | 0 | 0 | 0 | 0 |
| (DSQ) | | | | | | | |

TABLE 8
DNA quantity points assigned per tape

| | | Average | | | | | Median |
|---|---|---|---|---|---|---|---|
| Tape | Average | yield | Median | Top | Above | Above | yield |
| prototype | yield | points | yield | score | 75% | 50% | points |
| T1 | 112.40 | 0 | 52.15 | 0 | 0 | 0 | 0 |
| T2 | 224.34 | 1 | 48.825 | 0 | 0 | 0 | 0 |
| T3 | 106.39 | 0 | 42.7 | 0 | 0 | 0 | 0 |
| T4 | 179.14 | 1 | 106.05 | 0 | 0 | 1 | 1 |
| T5 | 119.50 | 0 | 88.375 | 0 | 0 | 1 | 1 |
| T6 | 718.47 | 1 | 99.225 | 0 | 0 | 1 | 1 |
| T7 | 774.47 | 1 | 139.475 | 0 | 2 | 1 | 3 |
| T8 | 281.27 | 1 | 85.225 | 0 | 0 | 0 | 0 |
| T9 | 277.29 | 1 | 121.975 | 0 | 2 | 1 | 3 |
| T10 | 327.64 | 1 | 58.275 | 0 | 0 | 0 | 0 |
| T11 | 307.79 | 1 | 107.625 | 0 | 2 | 1 | 3 |
| T12 | 572.32 | 1 | 164.5 | 1 | 2 | 1 | 4 |
| T13 | 201.64 | 1 | 81.55 | 0 | 0 | 0 | 0 |
| (CC) | | | | | | | |
| T14 | 131.47 | 0 | 36.575 | 0 | 0 | 0 | 0 |
| (DSQ) | | | | | | | |

ii. RNA Integrity and Fragment Size Distribution

RNA integrity was determined after the capillary electrophoresis and detection of RNA fragments on the Bioanalyzer2100 instrument (Agilent Technologies, Santa Clara, Calif.). RNA integrity number (RIN) and the percentage of fragments larger than 200 bp were used to assign points to each tape. RIN is presented on a scale of 1 to 10, with 1 being completely degraded RNA and 10 being completely intact RNA.

FIG. 16F illustrates a visual representation of the RNA electropherogram of Subject 7, showing results for tapes 5-14. Intensity of the bands corelates with yield. Subject 7 displayed higher than average RNA integrity. The prominent RNA species on the electropherogram are the ribosomal RNA 18S and 28S; the intensity of the bands is correlated to the quantity of RNA (FIG. 16F). The Bioanalyzer2100 software evaluates multiple aspects of the RNA electropherogram to determine the RIN number, including the ratio between 28S:18S14. The percentage of fragments larger than 200 bp (DV200) is used to inform on the handling of RNA during the cDNA library construction step in the preparation of samples for next generation sequencing. A size of approximately 300 bp is targeted during the library construction, thus a high percentage of fragments above 200 bp is necessary to insure capturing most of the representative RNA species. For each tape, numbers of subjects that had RIN above 4 and those that had DV200 above 50% were summed; highest value received 2 points, second highest 1 point and third highest 0.5 points (Table 9).

TABLE 9
RNA quality points assigned per tape

| | N of subjects | N of subjects | | RNA |
|---|---|---|---|---|
| Tape | with | with | | quality |
| prototype | RIN >4 | DV200 >50% | Sum | points |
| T1 | 4 | 10 | 14 | 0.5 |
| T2 | 3 | 5 | 8 | 0 |
| T3 | 2 | 3 | 5 | 0 |
| T4 | 5 | 5 | 10 | 0 |
| T5 | 5 | 6 | 11 | 0 |
| T6 | 6 | 9 | 15 | 1 |
| T7 | 7 | 9 | 16 | 2 |
| T8 | 5 | 7 | 12 | 0 |
| T9 | 4 | 4 | 8 | 0 |
| T10 | 4 | 6 | 10 | 0 |
| T11 | 6 | 9 | 15 | 1 |
| T12 | 7 | 8 | 15 | 1 |
| T13 (CC) | 5 | 6 | 11 | 0 |
| T14 (DSQ) | 2 | 9 | 11 | 0 |

iii. Consistency of Yield Across Subjects

FIG. 16G illustrates a bar graph showing differences in average yields between different subjects. Group one-way ANOVA (non-parametric Kruskall-Wallis test) yields a p-value of <0.0001. Nucleic acid yields from adhesive tapes collected from different individuals are a significant and known source of variability in a given subject cohort (FIG. 16G). For each subject, the total nucleic acid amounts were sorted in a descending order from the highest to the lowest and the top three tapes were recorded. Each tape was scored for consistency of performance by determining how frequently it featured as one of the top three highest yielding tapes across all subjects. Points were assigned as follows: the tape appearing in most of the subjects was assigned 3 points, the second and third best were assigned 2 and 1 points, respectively (Table 10).

TABLE 10
Points for performance consistency assigned per tape

| RNA | | | DNA | | |
|---|---|---|---|---|---|
| Tape | Top 3 | | Tape | Top 3 | |
| proto- | in N of | | proto- | in N of | |
| type | subjects | Points | type | subjects | Points |
| T1 | 3 | 0 | T1 | 0 | 0 |
| T2 | 4 | 0 | T2 | 3 | 0 |
| T3 | 3 | 0 | T3 | 1 | 0 |
| T4 | 7 | 2 | T4 | 7 | 2 |
| T5 | 5 | 0 | T5 | 1 | 0 |
| T6 | 5 | 0 | T6 | 7 | 2 |
| T7 | 8 | 3 | T7 | 7 | 2 |
| T8 | 6 | 1 | T8 | 7 | 2 |
| T9 | 3 | 0 | T9 | 4 | 0 |
| T10 | 3 | 0 | T10 | 6 | 1 |
| T11 | 4 | 0 | T11 | 6 | 1 |
| T12 | 8 | 3 | T12 | 9 | 3 |
| T13 (CC) | 3 | 0 | T13 | 4 | 0 |
| T14 (DSQ) | 1 | 0 | T14 | 1 | 0 |

iv. Percentage of Samples of Sufficient Quantity to Run a Melanoma Assay

All extracted samples were evaluated for sufficient quantity to run a melanoma assay by setting the cutoff of the total yield at 75 pg. Samples with a total yield of 75 pg or higher were considered acceptable for the assay; samples with a yield lower than 75 pg were labelled as QNS (quantity not sufficient). Median QNS rate was 17% (range 0-33%). Points were assigned as follows: Samples with a QNS rate lower than 10% were given 2 points; those with no QNS were assigned an additional point (Table 11).

Points assigned for RNA average quantity, median quantity, quality, consistency across subjects and melanoma assay QNS rates were summed to give a final RNA score for each tape. DNA points assigned for average quantity, median quantity and consistency across subjects were also summed to give a final DNA score for each tape.

TABLE 11
QNS rates and assigned points

| Tape | % | |
|---|---|---|
| prototype | QNS | Points |
| T1 | 29% | 0 |
| T2 | 24% | 0 |
| T3 | 29% | 0 |
| T4 | 5% | 2 |
| T5 | 10% | 2 |
| T6 | 0% | 3 |
| T7 | 14% | 0 |
| T8 | 14% | 0 |
| T9 | 19% | 0 |
| T10 | 29% | 0 |
| T11 | 14% | 0 |
| T12 | 14% | 0 |
| T13 (CC) | 19% | 0 |
| T14 | 33% | 0 |
| (DSQ) | | |

### 4. Scores

All assigned points were summed to give a final score for each tape. Table 12A shows the ranking of the tapes from the most to the least performing. Median threshold is shown by the bold line. Top performers are in bold.

TABLE 12A
Final scores and ranks of all 14 tape prototypes

| | | Barrier | | | | Final |
|---|---|---|---|---|---|---|
| Tape | Erythema | function | Discomfort | DNA | RNA | score |
| T12 | 0 | 0 | 0 | 8 | 9 | 17 |
| T7 | 0 | 0 | 0 | 6 | 6 | 12 |
| T4 | 2 | 0 | 0 | 4 | 5 | 11 |
| T6 | 1 | 0 | 0 | 4 | 5 | 10 |
| T11 | 1 | 0 | 0 | 5 | 4 | 10 |
| T5 | 1 | 0 | 0 | 1 | 5 | 7 |
| T9 | 0 | 0 | 0 | 4 | 3 | 7 |
| T8 | 0 | 0 | 0 | 3 | 2 | 5 |
| T13 | 2 | 0 | 0 | 1 | 2 | 5 |
| (CC) | | | | | | |
| T2 | 1 | 0 | 0 | 1 | 2 | 4 |
| T10 | 0 | 0 | 0 | 2 | 1 | 3 |
| T3 | 2 | 0 | 0 | 0 | 0 | 2 |
| T14 | 2 | 0 | 0 | 0 | 0 | 2 |
| (DSQ) | | | | | | |
| T1 | 1 | 0 | 0 | 0 | 0.5 | 1.5 |

### Conclusions

Results of the study described above suggest good tolerability of sample collectors and collection systems disclosed herein by the subjects and maintenance of non-invasive properties when sampling the upper back skin with four consecutive tapes. Sample collectors and collection systems disclosed herein have consistently shown higher nucleic acid yields than prior devices. Sample collectors and collection systems disclosed herein did not generate any skip pattens during the sampling procedure.

Melanoma Cell Line Testing T12 and T7 v. T14 and T13

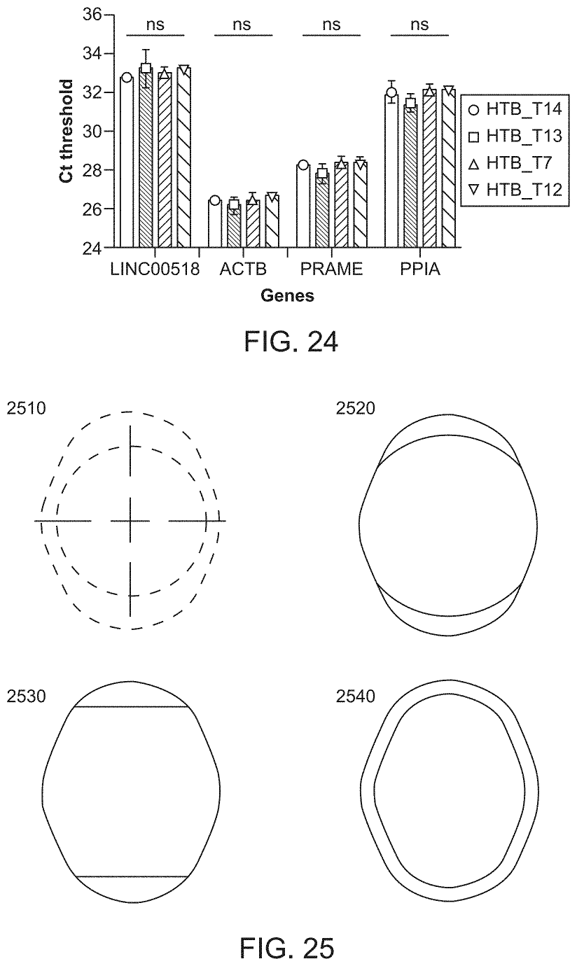

Extracted samples from T12, T7, T14, and T13 were evaluated to determine tape material interference with detection of gene expression in Applicant's human melanoma cell line assay (HTB). FIG. 24 shows a comparison of each sample for four different genes LINC00518, ACTB, PRAME, and PPIA as a function of the cycle threshold (Ct). Cycle threshold levels are inversely proportional to the amount of the nucleic acid in the sample (i.e., the lower the Ct level the greater the amount of target nucleic acid in the sample). As shown, the cycle threshold for each of the tapes is comparable suggesting that, while the total nucleic acid content extracted is higher for T7 and T12, all samples tested are sufficient for detection of various genes. Table 12B below shows upper and lower bounds for the comparison of the thresholds for each of the comparison groups.

TABLE 12B
DMT Assay HTB Melanoma Cells

| Comparison Groups | T14 v. T13 | T13 v. T7 | T13 v. T12 |
|---|---|---|---|
| Lower bound (TOST) | −1.500 | −1.500 | −1.500 |
| Lower bound (90%) | −0.859 | −1.158 | −1.033 |
| Upper bound (90%) | 1.012 | 0.685 | 0.806 |
| Upper bound (TOST) | 1.500 | 1.500 | 1.500 |
| p-value | 0.007 | 0.012 | 0.007 |

### Example 9: Extractables/Leachables Study

### Summary

Medical grade adhesives were investigated using instrumental and analytical techniques including FT-IR, GC-MS, Extractables and Leachables (E&L) screening study, 180° peel adhesion test and appropriate metrology.

According to the E&L analysis, all tape samples from FLEXcon and Lamart generally surpass the performance of ARcare 90068. 3M adhesives show very poor E&L performance.

GC-MS analysis and gravimetric analysis reveal that some tapes are more inert to ethanol/water co-solvent systems, which are used during DNA extraction. Flexcon H-778, Flexcon H-566, Lamart 160-49, and Lamart H-52 were shown to demonstrate excellent E&L profile while having moderate to high adhesion strengths that are advantageous for the application (i.e. ˜10 N/inch and above). These adhesives can be applied or transferred onto medical grade TPU, LDPE or non-woven type of materials of different colors and transparency/opacity.

The selection of the backing layer is shown to be an important variable for performance. The role of substrate on formation of discontinuities along the peel line can be predicted through evaluation of mechanisms that govern the buckling instabilities occurring on thin structures/layers. In that regard, 4-6 mil thick TPU or LDPE were found to suit the application better than the 3 mil TPU used at ARcare 90068 product. Thicker tapes will reduce the frequency of interruptions and line formation at least by 25-50% chance.

Overall, the different versions of the tapes can be customized using the aforementioned materials and the tape manufacturer as listed above. Preliminary results suggest use of 1 mil thick FLEXcon H-778 and H-566 adhesives on 3 mil and 5 mil (or 4 mil vs. 6 mil) thick medical grade TPU films, and slit the coated films capped with a release liner to 1″ wide rolls of samples. However, different variations of the films may be used.

### FTIR Compositional Analysis

Each layer of the tape, after properly separated, was placed on the ATR crystal to attain accurate signal for verification of the resin systems.

Instrument Parameters—Instrument: Perkin Elmer Spectrum 65 FT-IR with Universal ATR Sampling Arm. Spectral Range: 3700-600 cm⁻¹. Number of Scans: 8 scans. Resolution: 4 cm⁻¹.

The composition and thickness of adhesive and backing layer from each sample was accurately identified by FT-IR (ATR crystal) and calibrated absolute digital caliper (Mitutoyo American Corp.) using appropriate separation method as shown in Table 13.

TABLE 13
Description of Tape Samples

| | | | Backing | | |
|---|---|---|---|---|---|
| | | Product | Com- | Adhesive | |
| # | Vendor | Name | position | Composition | Features |
| 01 | 3M | 1527 | 1 mil Acrylic | 5 mil | Medical/ |
| | | | | EVA Polymer | Surgical |
| | | | | | use |
| 02 | 3M | 9830 | 1 mil Acrylic | 2 mil | Medical/ |
| | | | | Polyethylene | Surgical |
| | | | | | use |
| 03 | 3M | 9832F | 1 mil Acrylic | 1 mil | Medical/ |
| | | | | Polyurethane | Surgical |
| | | | | | use |
| 04 | Flexcon | H-778 | 1 mil Acrylic | 18 mil Aperture | Approved for |
| | | | | Polyester Fabric | skin contact |
| 05 | Flexcon | H-566 | 1 mil Acrylic | 18 mil Aperture | Approved for |
| | | | | Polyester Fabric | skin contact |
| 06 | Flexcon | H-520 | 2 mil Acrylic | 18 mil Aperture | Approved for |
| | | | | Polyester Fabric | skin contact |
| 07 | Lamart | 160-49 | 1 mil Acrylic | 4 mil | Medical use |
| | | | | Polyester PET | |
| 08 | Lamart | 216-47 | 1 mil Acrylic | 4 mil | Medical use |
| | | | | Polyester PET | |
| 09 | Lamart | 290-51 | 1 mil Acrylic | 4 mil | Medical use |
| | | | | Polyester PET | |
| 10 | Lamart | A64 | 1 mil Acrylic | 4 mil | Medical use |
| | | | | Polyester PET | |
| 11 | Lamart | H-72 | 1 mil Rubber | 4 mil | Medical use |
| | | | | Polyester PET | |
| 12 | Lamart | H-52 | 1 mil Rubber | 5 mil Silicone | Medical use |
| | | | | Release Liner | |
| 13 | Applicant | Arcare | 2 mil Rubber | 3 mil | Medical use |
| | Com- | 90068 | | Polyester TPU | |
| | parator | | | | |

Extractables and Leachables (E&L) Analysis with GC-MS and Gravimetric Analysis

Preparation of Tape Samples and Extractions: The tape samples as a whole were prepared for GC-MS analysis by first cutting a piece of the tape (5 cm×5 cm) with a clean razor blade and placing it into a separate glass scintillation vial. 40 mL of 20% ethanol and 80% ethanol (which are used for RNA isolation) was added to each vial separately using a Class A graduated cylinder. The vials were allowed to stir for about 3 hours and 1 ml aliquots of each extraction were collected and diluted with 9 ml of Methanol. The diluted solution was then transferred into GC-MS vials.

Instrument Parameters: Instrument: Agilent 6890N GC with Agilent 5973 Mass Selective Detector (MSD). Column: DB-5MS, 30 m×0.25 mm×0.25 μm and 30 m×0.25 mm×1.0 μm. Temperature Program: Initial at 35° C., ramp to 300° C. at 10° C./min.

Results: Interference (volatile residuals, additives, fillers, binders, etc.) with RT-PCR test that could be present in sample tapes for this skin stripping application was analyzed by GC-MS extraction and gravimetric analysis using Ethanol and Isopropanol (which are used for RNA isolation).

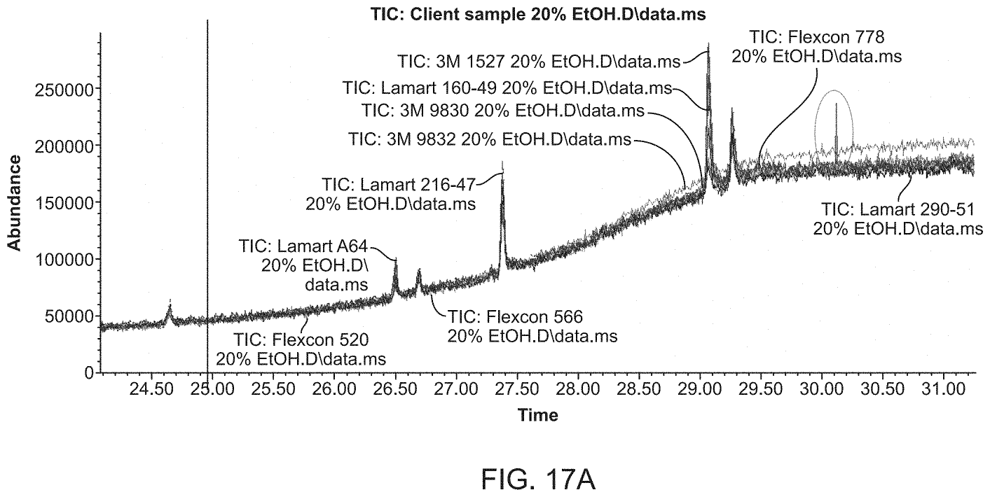

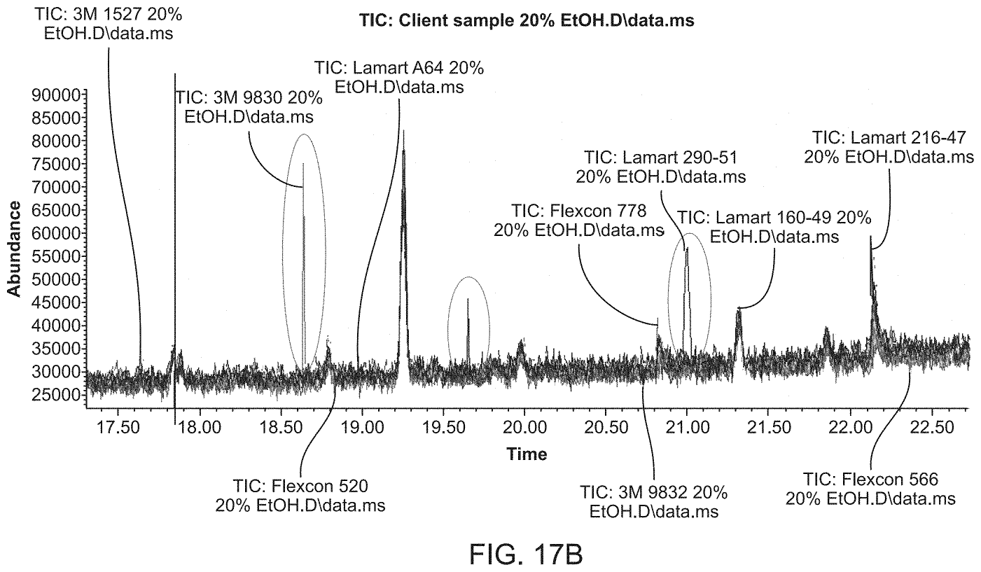

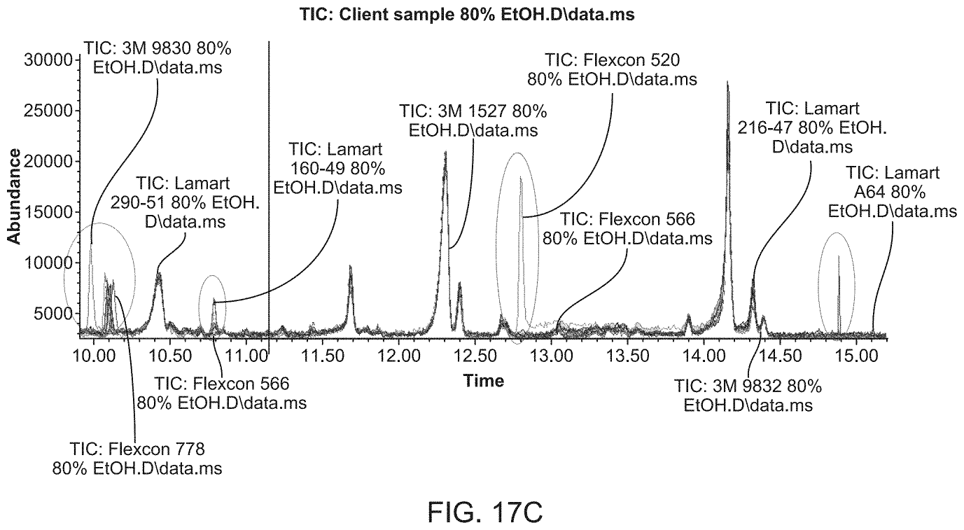

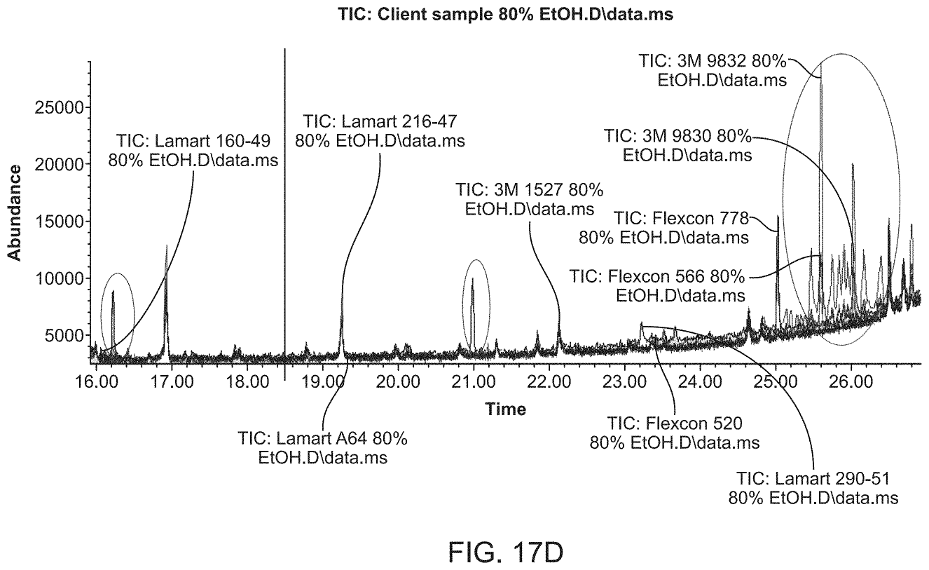

The GC-MS chromatogram of 20% and 80% Ethanol extract from each tape sample was overlaid with the chromatogram of the sample (ARcare 90068 from Adhesive research) for benchmarking purpose. FIG. 17A illustrates an overlaid GC-MS chromatogram of 20% ethanol extractions from samples (Circled: Sample 2 distinct peak at around 31 min). The x-axis is labeled 24.50 to 31.00 at 0.5 minute intervals. FIG. 17B illustrates an overlaid GC-MS chromatogram of 20% ethanol extractions from samples (Circled: Sample 3 at 18.6 min, Sample 10 at 19.6 min and Sample 1 at 21 min). The x-axis is labeled 17.50 to 22.50 at 0.5 minute intervals. FIG. 17C illustrates an overlaid GC-MS chromatogram of 80% ethanol extractions from samples. Circled: All samples at 10.1 min except for sample 1 (confirmed by individual overlay with Sample 1). Sample 3, Sample 7, Sample 8, and Sample 9 at 10.8 min (confirmed by individual overlay with Sample 1), Sample 5 at 12.8 min and Sample 8 at 14.9 min. The x-axis is labeled 10.00 to 15.00 at 0.5 minute intervals. FIG. 17D illustrates an overlaid GC-MS chromatogram of 80% ethanol extractions from samples. (Circled: Sample 1 at 16.2 min and 21 min, Sample 2, Sample 3, Sample 4 and Sample 6 at 25.5-26.5 min (confirmed by individual overlay with Sample 1). The x-axis is labeled 16.00 to 26.00 at 0.5 minute intervals.

E&L ingredient's ID and concentration of each sample were carefully identified and quantified by GC-MS reference library and peak area along with gravimetric study results as provided in the following Table 14.

TABLE 14
Results of GC-MS and Gravimetric E&L Analysis

| | | 20% Ethanol Extraction | | | | 80% Ethanol Extraction | | | |
|---|---|---|---|---|---|---|---|---|---|
| | | Peak | | Peak | Total | Peak | | Peak | Total |
| | | Positions | | Area | E&L | Positions | | Area | E&L |
| Vendor | Product | (min) | Peak ID | (counts) | (ppm) | (min) | Peak ID | (counts) | (ppm) |
| 3M | 1527 | Singlet at | Siloxane | 290,035 | 0 | Singlet | Hexylene | 780,353 | 599 |
| | | 31 | | | | at 10.1 | glycol | | |
| | | | | | | Multiplet | Unidentified oil | 4,139,655 | |
| | | | | | | at | | | |
| | | | | | | 25.5-26.1 | | | |
| 3M | 9830 | Singlet at | Carbohydrazide | 239,507 | 0 | Singlet | Hexylene | 1,471,684 | 793 |
| | | 18.6 | | | | at 10.1 | glycol | | |
| | | | | | | Multiplet | Unidentified oil | 3,652,280 | |
| | | | | | | at | | | |
| | | | | | | 25.5-26.1 | | | |
| 3M | 9832F | N/A | N/A | N/A | 0 | Singlet | Hexylene | 736,098 | 1396 |
| | | | | | | at 10.1 | glycol | | |
| | | | | | | Multiplet | Unidentified oil | 15,240,927 | |
| | | | | | | at | | | |
| | | | | | | 25.5-26.1 | | | |
| Flexcon | H-778 | N/A | N/A | N/A | 0 | Singlet | Hexylene | 922,902 | 202 |
| | | | | | | at 10.1 | glycol | | |
| Flexcon | H-566 | N/A | N/A | N/A | 0 | Singlet | Hexylene | 956,875 | 198 |
| | | | | | | at 10.1 | glycol | | |
| | | | | | | Singlet | Unidentified oil | 621,378 | |
| | | | | | | at 25.5 | | | |
| Flexcon | H-520 | N/A | N/A | N/A | 0 | Singlet | Hexylene | 950,207 | 197 |
| | | | | | | at 10.1 | glycol | | |
| | | | | | | Singlet | Ethyl hexanol | 2,418,063 | |
| | | | | | | at 12.8 | | | |
| Lamart | 160-49 | N/A | N/A | N/A | 0 | Singlet | Hexylene | 812,391 | 199 |
| | | | | | | at 10.1 | glycol | | |
| | | | | | | Singlet | Carbohydrazide | 404,029 | |
| | | | | | | at 14.9 | | | |
| Lamart | 216-47 | N/A | N/A | N/A | 0 | Singlet | Hexylene | 661,976 | 200 |
| | | | | | | at 10.1 | glycol | | |
| Lamart | 290-51 | Singlet | Carbohydrazide | 92,742 | 0 | Singlet | Hexylene | 845,344 | 200 |
| | | at 19.6 | | | | at 10.1 | glycol | | |
| Lamart | A64 | N/A | N/A | N/A | 0 | Singlet | Hexylene | 823,761 | 198 |
| | | | | | | at 10.1 | glycol | | |
| Lamart | H-52 | N/A | N/A | N/A | 0 | N/A | N/A | N/A | 219 |
| Lamart | H-72 | N/A | N/A | N/A | 0 | N/A | N/A | N/A | 199 |
| A.R. | ARcare | Singlet | Unidentified | 397,527 | 0 | Singlet | Low MW | 1,055,007 | 599 |
| | 90068 | at 21 | Acid | | | at 16.2 | Diester | | |
| | | | Carboxylic | | | Singlet | Low MW | 1,245,290 | |
| | | | | | | at 21 | Diester | | |

This classification shows adhesive samples which are considered as promising alternatives compared to other samples with respect to the unnecessary extractables released to the analysis solution.

### Physical and Mechanical Properties of Adhesive and Backing Film

Tensile Testing—Instrument Parameters: Instrument: Labthink XLW Auto tensile tester with load cell capacity of 500N. Test standard: ASTM D3330 (Peel Adhesion), ASTM D882 (Elastic Properties)

Peel Adhesion of adhesive: Peel adhesion strength of different sample tapes were benchmarked and measured in triplicate per the ASTM D3330 180° peel adhesion standard method using an XLW (EC) Auto Tensile Tester (Labthink Instrument Inc). The results are summarized in Table 15 below:

TABLE 15
Results of ASTM D3330 1800 Peel Adhesion Test

| | | | | Peel Adhesion |
|---|---|---|---|---|
| | Adhesive/ | | | Strength |
| | Tape | Product | Adhesive | (Newton/25 mm |
| # | Vendor | Name | Specs | Width) |
| 01 | 3M | 1527 | 1 mil Acrylic | 2.8 |
| 02 | 3M | 9830 | 1 mil Acrylic | 3.6 |
| 03 | 3M | 9832F | 1 mil Acrylic | 6.8 |
| 04 | Flexcon | H-778 | 1 mil Acrylic | 15.1 |
| 05 | Flexcon | H-566 | 1 mil Acrylic | 11.9 |
| 06 | Flexcon | H-520 | 2 mil Acrylic | 6.1 |
| 07 | Lamart | 160-49 | 1 mil Acrylic | 13.2 |
| 08 | Lamart | 216-47 | 1 mil Acrylic | 1.5 |
| 09 | Lamart | 290-51 | 1 mil Acrylic | 7.6 |
| 10 | Lamart | A 64 | 1 mil Acrylic | 3.3 |
| 11 | Lamart | H-72 | 1 mil Rubber | 9.8 |
| 12 | Lamart | H-52 | 1 mil Rubber | 19.9 |
| 13 | Adhesives | Arcare 90068 | 2 mil Rubber | 17.4 |
| (CC) | Research | | | |

Flexcon H-778, Flexcon H-566, Lamart H-52 and Lamart 160-49 adhesives provided enough peel force capacity to qualify for the application.

Elastic modulus of Backing Film: flexibility of different sample tapes were benchmarked and quantified in triplicate with ASTM D882 tensile test and elongation rate standard methods using XLW (EC) Auto Tensile Tester (Labthink Instrument Inc). A summary of elastic moduli measurements for the backing layer is presented in Table 16.

TABLE 16
An Illustrative ASTM D882 Analysis of Backing Layers

| | | Com- | | Thick- | Elastic |
|---|---|---|---|---|---|
| Vendor | Product | position | Color | ness | Modulus |
| SWM | ST-1522 | TPU Ether | Clear | 3 mil | 1,601 psi |
| PFC | 78000450M | TPU Ether | Light Matte | 4 mil | 1,207 psi |
| | | | Clear | | |
| SWM | 18405 | TPU Ether | Frosted | 5 mil | 1,140 psi |
| McMaster- | Flexible | Polyethylene | Semi clear | 2 mil | 1,417 psi |
| Carr | LDPE | | white | | |
| McMaster- | Flexible | Polyethylene | Semi clear | 4 mil | 1,288 psi |
| Carr | LDPE | | white | | |
| McMaster- | Flexible | Polyethylene | Semi clear | 6 mil | 939 psi |
| Carr | LDPE | | white | | |

In general, medical grade TPU and LDPE is offered at different ranges clarity/opacity, colors, and thicknesses. The elastic modulus of TPU and LDPE imparts flexibility and softness for the end user. However, other stiffer resins can be also use as these materials would be more robust against deformation during the pull and retention on skin. A stiffer material, however, may not feel as soft on the skin.

Wrinkling Phenomena Vs. Smooth Peel

Background—Micro stick-slips are believed to be controlled by a release of bending energy in the tape near the separation front. The periodicity of the slip structures is governed by the bending stiffness of the backing layer and the peel angle. The amplitude and the periodicity of the structures are proportional to each other. Moreover, the amplitude A scales as the following equation, where A is the Amplitude of the microscopic stick-slip, λ is the periodicity of the microscopic stick-slip, B is the bending stiffness, and θ is the peel angle.

A˜[2B/(1−cos θ)]^{1/6} and A˜λ

In the equation above, the bending stiffness may be equivalent to B=Eₜt³/[12(1−ν²)], where Eₜ is the elastic modulus of the tape/backing layer, ν is Poisson's ration of tape/backing layer, and t is the thickness of tape/backing layer.

Results—It was found that in a typical monotonic-rate pull test, the longer the duration of the “stick,” the more distance was covered during the following slip. The relationship between these two quantities was not a direct proportionality: the slip distance was proportional to the cubic root of the time between slips. At the moment of slip, stored up potential energy is converted to kinetic energy. This description of the energy balance leads to an equation showing the cube-root relationship between the microslip distance and duration. In that regard, slower peels were also found to facilitate less frequent stick-slips.

It was found that the thickness of the backing sheet is an important design parameter to decrease the frequency of the slip lines. According to the equation above, the frequency of the stick-slip patterns should decrease with the square root of the tape thickness. The modulus of elasticity of the backing sheet weakly governs the features by the square root of the cubic root, which provides an exponent of ⅙, (i.e. λ˜Eₜ^{1/6}).

Further, both stick-slip and wrinkling phenomena may govern the periodicity and the shape of the wrinkles formed in the sample collectors and collection systems of the present disclosure. The wave-like periodic texture of wrinkles that is generated by an adhesive tape on skin is governed by elasticity of the skin, elasticity of the backing layer, strength of the adhesive (tension exerted onto skin) and geometric parameters such as width and thickness of the tape.

The wavelength (λ) or periodicity/frequency of the lines are dictated by the below equation, where λ is the periodicity of the macroscopic wrinkles, Eₜ is the elastic modulus of tape/backing layer, Eₛ is the elastic modulus of skin, and t is the thickness of tape/backing layer.

$$λ = 2 π * {( \frac{{E}_{t}}{3 {E}_{3}} )}^{\frac{1}{3}} * t$$

The elastic behavior of skin should vary with age, physical condition of patient, and location of the skin on body. However, the tape properties can be customized.

It was found that if one chooses Polyurethane or Low density polyethylene backing film, higher thicknesses tend to mitigate wavy structures forming on the skin. Based on the above, the frequency of the features may be linearly proportional to the thickness of the backing sheet.

The amplitude (i.e., out of plane deformation of the skin) may be governed by the following equation, and just like stick-slip patterns, it may be proportional to the wavelength of the patterns, where A is the amplitude, Fₜ is the adhesive force per width of the tape, Lₜ is the length of tape, and λ is the wavelength of features formed.

$$A ∝ {( \frac{{F}_{t}}{{L}_{t}} )}^{\frac{1}{2}} * λ$$

It was found that thicker backing layers are likely to reduce the amplitude and wavelength of features formed on skin (e.g. wrinkles, and stick-slip structures). These phenomena are further examined in Example 10.

Description of Instrumentation Used—Fourier Transform Infrared Spectrometer (FTIR): FT-IR technology was used to obtain molecular fingerprints of unknown compounds, using transmittance measurements captured after firing an infrared beam at the sample compounds. Unknown fingerprints are then searched against a database of over 230,000 known FT-IR spectra in order to develop a conclusive identification. A Perkin Elmer Spectrum 65 FT-IR is equipped with an Attenuated Total Reflectance (ATR) accessory to facilitate use of diamond cell technology to test solid, liquid, or gas state samples in their natural state at much lower sample sizes. Gas Chromatography/Mass Spectrometry (GC-MS): GC/MS testing allows for the analysis of samples along multiple dimensions of chemical properties, providing specific identification of the different compounds separated during the GC analysis. The gas chromatograph separates a complex mixture into its individual components and delivers each one to the mass spectrometer. This analysis generates a chromatogram consisting of different peaks, one for each component of a mixture. The area of each peak is used to measure quantity. GC/MS analysis can be used both for qualitative and quantitative determinations of chemical composition. Labthink© XLW (EC) Auto Tensile Tester was used determine the tensile, peeling, tearing, heat sealing, adhesive, piercing, opening, low speed unwrapping and pulling force of the plastic film, composite material, flexible packaging material, plastic tube, adhesives, adhesive tapes, medical plasters, release paper, protective films, combination caps, aluminum foils, diaphragm, back sheets, non-woven fabrics, rubber, and paper etc.

### Example 10: Mechanical Property Study

### Summary

The mechanical properties of skin sample collectors and collection systems of the present disclosure were investigated using instrumental techniques including ASTM D3121 tack and ASTM D3330 180° peel adhesion test, as well as testing for comfort and wrinkles on skin. According to the tack and 180° peel adhesion test, all films showed moderate to high tack and adhesion force sufficient for the application (i.e. ˜10 N/inch and above). The study also showed that thicker backing films provided slightly higher tack and peel strength values. The test for skip pattern and wrinkling analysis showed that 6 mil thick backing sheets were found to suit the application better than the 4 mil films which created more wrinkles, and 5 mil films which tended to create more skip patterns. Thicker tapes were shown to reduce the frequency of interruptions and line formation. Overall data indicated that Tape 05 and 06 could be desirable options for the application, provided that they qualify during diagnosis process.

### Materials

Medical grade (2-ethylhexyl acrylate polymer) adhesives with EVA backing sheets were customized and manufactured as in Table 17:

TABLE 17
Tape/Sample Summary

| | Backing Tape | Adhesive ID | | |
|---|---|---|---|---|
| Sample | Product ID from | from Lamart | Thickness (mil) | |
| # | Wiman Corporation | Corporation | Adhesive | Backing |
| 01 | .004 × 12″ PEO3000 | Lamart 160-49 | 1.0 | 4 |
| | Clear Matte mPE film | | | |
| 02 | .005 × 12″ PEO3000 | Lamart 160-49 | 1.0 | 5 |
| | Clear Matte mPE film | | | |
| 03 | .006 × 12″ PEO3000 | Lamart 160-49 | 1.0 | 6 |
| | Clear Matte mPE film | | | |
| 04 | .004 × 12″ PEO3000 | Lamart 160-49 | 1.5 | 4 |
| | Clear Matte mPE film | | | |
| 05 | .005 × 12″ PEO3000 | Lamart 160-49 | 1.5 | 5 |
| | Clear Matte mPE film | | | |
| 06 | .006 × 12″ PEO3000 | Lamart 160-49 | 1.5 | 6 |
| | Clear Matte mPE film | | | |
| 07 | .004 × 12″ PEO3000 | Lamart 160-49 | 2.0 | 4 |
| | Clear Matte mPE film | | | |
| 08 | .005 × 12″ PEO3000 | Lamart 160-49 | 2.0 | 5 |
| | Clear Matte mPE film | | | |
| 09 | .006 × 12″ PEO3000 | Lamart 160-49 | 2.0 | 6 |
| | Clear Matte mPE film | | | |
| 10 | .004 × 12″ PEO3000 | Lamart 160-49 | 2.5 | 4 |
| | Clear Matte mPE film | | | |
| 11 | .005 × 12″ PEO3000 | Lamart 160-49 | 2.5 | 5 |
| | Clear Matte mPE film | | | |
| 12 | .006 × 12″ PEO3000 | Lamart 160-49 | 2.5 | 6 |
| | Clear Matte mPE film | | | |

Backing sheets of 4, 5 and 6 mils thick films without anti-block additives were tested for the trials. Versions with anti-block additives may be used and may even be smoother that the films tested herein.

### Peel Adhesion

180° peel adhesion Testing Instrument Parameters—Instrument: Labthink XLW Auto tensile tester with load cell capacity of 500 N. Test standard: ASTM D3330 (180° Peel Adhesion). Pull rates: 3.94 in/min and 7.87 in/min

Adhesion strength of the tape samples were measured in duplicate at 3.94 in/min and 7.87 in/min pull rates per the 180° peel adhesion standard method described in ASTM D3330 using XLW (EC) Auto Tensile Tester (Labthink Instrument Inc). The results are summarized in Table 18 below:

TABLE 18
Results of ASTM D3330 180° Peel Adhesion Test

| | | | | | | | Avg. |
|---|---|---|---|---|---|---|---|
| | | | Peel | Peel | Avg. | Avg. | Peel |
| | | | Strength | Strength | Peel | Peel | Strength |
| | Thickness (mil) | | #1 | #2 | Strength | Strength | (N/25 |
| ID | Adhesive | Backing | (Ibf/in) | (Ibf/in) | (Ibf/in) | (N/cm) | mm) |
| 01 | 1.0 | 4 | 1.77 | 1.62 | 1.69 | 3.01 | 7.66 |
| 02 | 1.0 | 5 | 2.29 | 2.43 | 2.36 | 4.20 | 10.67 |
| 03 | 1.0 | 6 | 3.06 | 1.55 | 2.30 | 4.10 | 10.42 |
| 04 | 1.5 | 4 | 3.27 | 3.76 | 3.52 | 6.26 | 15.90 |
| 05 | 1.5 | 5 | 3.19 | 3.87 | 3.53 | 6.28 | 15.95 |
| 06 | 1.5 | 6 | 3.32 | 4.53 | 3.92 | 6.99 | 17.75 |
| 07 | 2.0 | 4 | 3.09 | 3.82 | 3.45 | 6.15 | 15.62 |
| 08 | 2.0 | 5 | 3.81 | 4.05 | 3.93 | 7.00 | 17.79 |
| 09 | 2.0 | 6 | 3.86 | 4.35 | 4.11 | 7.31 | 18.58 |
| 10 | 2.5 | 4 | 3.48 | 4.14 | 3.81 | 6.78 | 17.21 |
| 11 | 2.5 | 5 | 4.41 | 3.81 | 4.11 | 7.32 | 18.58 |
| 12 | 2.5 | 6 | 3.94 | 5.05 | 4.49 | 8.00 | 20.33 |

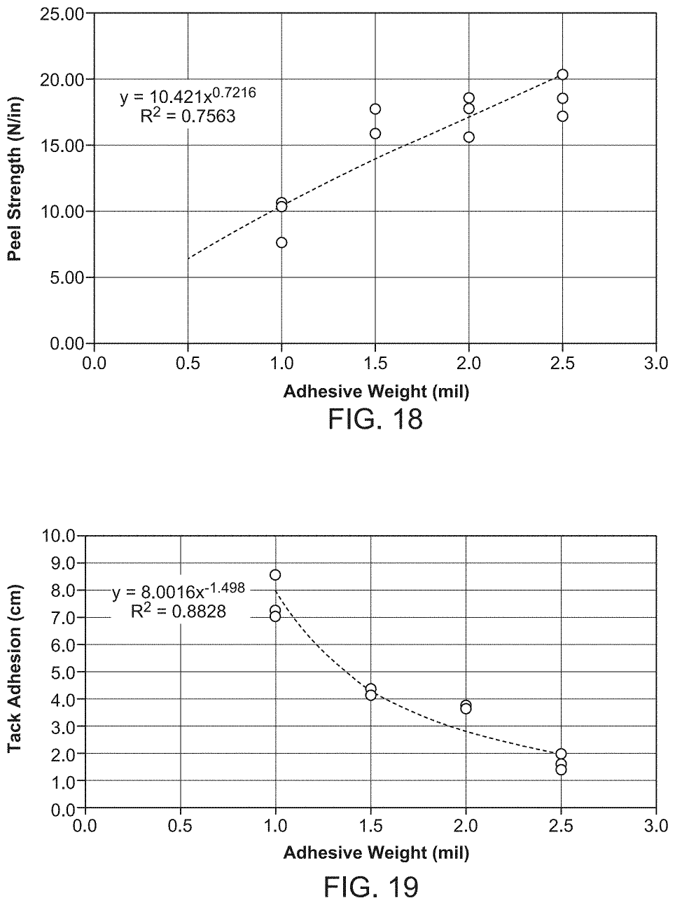

FIG. 18 illustrates a graph of peel strengths (N/in) as a function of adhesive thickness (mil). The trendline is labeled as y=10.421x^{0.7216}, R²=0.7563. In general, thicker adhesive coatings were measured to provide stronger adhesion to substrates.

2.0 mil and 2.5 mil adhesives on EVA films were observed to provide peel force comparable to the original tape. The thickness of the backing sheet was observed to slightly increase the peel force.

### Tack

Tack Testing Instrument Parameters—Instrument: ASTM.D3121.10—Rolling Ball Tack Tester Material Testing Technology wheeling, Ill. Test standard: ASTM D3121 (Tack of pressure-sensitive adhesives by rolling ball)

The American Society of Testing Materials describes tack as the ability of a material to adhere to a solid surface when brought into contact under very light pressure. Various testing procedures are described in ASTM D1878-61T. For example, rolling ball and related tests take into account the bond making and bond breaking processes. Rotating drum, toothed wheel, and other tests also fall into this category.

Tack of different sample tapes was evaluated in triplicate per the tack of pressure-sensitive adhesives standard method described in ASTM D3121 by rolling ball using a rolling ball tack tester (available from MTT, Material Testing Technology Wheeling, Ill.).

The tack results were measured by the distance the rolling ball travelled onto the tape and the results are summarized in Table 19 below:

TABLE 19
Results of ASTM D3121 tack test by rolling ball

| | | | Distance | Distance | Distance | Avg. |
|---|---|---|---|---|---|---|
| | | | ball | ball | ball | Distance |
| | | | travelled | travelled | travelled | ball |
| | Thickness (mil) | | Trial #1 | Trial #2 | Trial #3 | travelled |
| ID | Adhesive | Backing | (cm) | (cm) | (cm) | (cm) |
| 01 | 1.0 | 4 | 8.7 | 8.1 | 8.9 | 8.6 |
| 02 | 1.0 | 5 | 7.7 | 7.1 | 7.0 | 7.3 |
| 03 | 1.0 | 6 | 6.7 | 7.2 | 7.3 | 7.1 |
| 04 | 1.5 | 4 | 4.5 | 4.2 | 4.4 | 4.4 |
| 05 | 1.5 | 5 | 4.2 | 4.5 | 4.0 | 4.2 |
| 06 | 1.5 | 6 | 4.1 | 4.3 | 4.0 | 4.1 |
| 07 | 2.0 | 4 | 3.7 | 4.0 | 3.6 | 3.8 |
| 08 | 2.0 | 5 | 3.8 | 3.3 | 3.9 | 3.7 |
| 09 | 2.0 | 6 | 3.8 | 3.6 | 3.7 | 3.7 |
| 10 | 2.5 | 4 | 2.1 | 1.9 | 2.0 | 2.0 |
| 11 | 2.5 | 5 | 1.4 | 1.5 | 2.0 | 1.6 |
| 12 | 2.5 | 6 | 1.3 | 1.6 | 1.4 | 1.4 |

FIG. 19 illustrates a graph of tack adhesion (cm) as a function of adhesive thickness (mil). The trendline is labeled as y=8.0016x^{-1.498}, R²=0.8828. Similar to the peel strength results, tack gets stronger with the thickness of the adhesive layer increasing. The thickness of the backing sheet, again, was not observed to play a major role in tack behavior.

### Wrinkling and Comfort on Skin

Wrinkles are often observed when a harder layer gets into contact with a softer foundation. Without being bound by theory, wrinkles were observed in the results below because the relative stiffness of the tape is harder than the soft dermal layer. On flat surfaces, the tapes do not typically form wrinkles, but as soon as they are applied onto curved contours on body (such as joints) wrinkling was observed in some tapes.

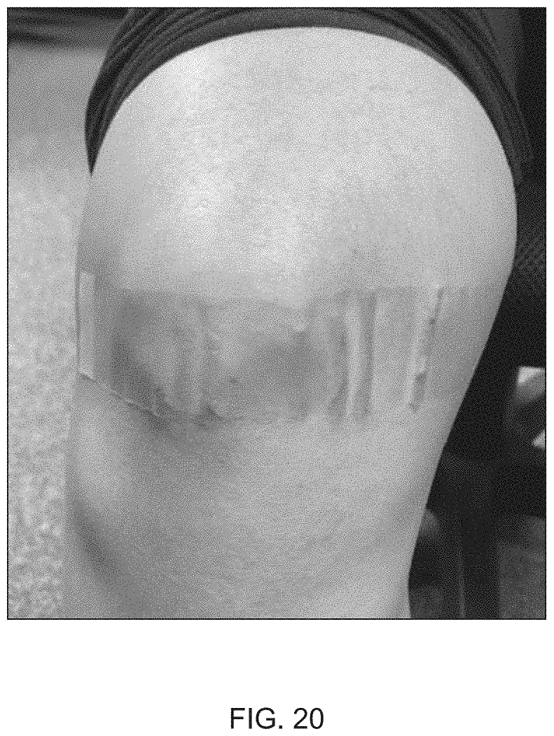

FIG. 20 illustrates an example of severe wrinkle formation on Tape 01 placed on the upper arm of Panelist #2. To test the wrinkling phenomenon and the number of wrinkles that may form on the skin, each tape was cut to 1″×5″ pieces. All these samples were gently placed on the knees of three different panelists. The knees are bended 90 degrees while the panelist was sifting on the chair. The number of wrinkles formed on the surface of the tapes were counted. The results are summarized in Table 20.

TABLE 20
Number of Wrinkles Counted on Each Tape

| | Thickness (mil) | | Panelist | Panelist | Panelist | Avg. # of |
|---|---|---|---|---|---|---|
| ID | Adhesive | Backing | 1 | 2 | 3 | Wrinkles |
| 01 | 1.0 | 4 | 5 | 14 | 8 | 9.0 |
| 02 | 1.0 | 5 | 0 | 4 | 3 | 2.3 |
| 03 | 1.0 | 6 | 0 | 4 | 3 | 2.3 |
| 04 | 1.5 | 4 | 1 | 7 | 7 | 5.0 |
| 05 | 1.5 | 5 | 3 | 5 | 4 | 4.0 |
| 06 | 1.5 | 6 | 3 | 7 | 0 | 3.3 |
| 07 | 2.0 | 4 | 4 | 11 | 6 | 6.9 |
| 08 | 2.0 | 5 | 8 | 7 | 6 | 7.0 |
| 09 | 2.0 | 6 | 3 | 7 | 4 | 4.6 |
| 10 | 2.5 | 4 | 0 | 11 | 10 | 7.0 |
| 11 | 2.5 | 5 | 5 | 9 | 3 | 5.7 |
| 12 | 2.5 | 6 | 4 | 7 | 3 | 4.7 |

As Tape 07 and 08 were peeled off, it was noted that there were macroscopic skip patterns formed on the adhesive layer. These patterns generally were not advantageous for consistent peeling, as it is a manifestation of lost mechanical energy due to peeling. A skip pattern analysis was not performed for all the samples, as this behavior was only noted for samples 07, 08 and 10.

After counting wrinkles, the panelists were asked to remove the tapes at a certain rate that they desire while their discomfort level with respect to removing a typical bad aid were asked to be rated (5: Hardest Band-Aid; 1: Softest Band-Aid they may consider). Table 21 shows a summary of the panelist ratings.

TABLE 21
Level of Discomfort during Peel-off Attempts

| | Thickness | Thickness | | | | Hardest (5); |
|---|---|---|---|---|---|---|
| | (mil) | (mil) | Panelist | Panelist | Panelist | Softest (1) |
| ID | Adhesive | Backing | 1 | 2 | 3 | Feel |
| 01 | 1.0 | 4 | 1 | 1 | 1 | 1.0 |
| 02 | 1.0 | 5 | 3 | 1 | 1 | 1.7 |
| 03 | 1.0 | 6 | 2 | 1 | 1 | 1.3 |
| 04 | 1.5 | 4 | 2 | 3 | 1 | 2.0 |
| 05 | 1.5 | 5 | 2 | 3 | 2 | 2.3 |
| 06 | 1.5 | 6 | 3 | 2 | 2 | 2.3 |
| 07 | 2.0 | 4 | 3 | 4 | 3 | 3.3 |
| 08 | 2.0 | 5 | 3 | 4 | 3 | 3.3 |
| 09 | 2.0 | 6 | 3 | 3 | 3 | 3.0 |
| 10 | 2.5 | 4 | 2 | 3 | 3 | 2.7 |
| 11 | 2.5 | 5 | 3 | 4 | 3 | 3.3 |
| 12 | 2.5 | 6 | 4 | 4 | 3 | 3.7 |

In general, the discomfort was not described by panelists as so painful as to stop using the taps, however, panelists were able to notice the differences from tape to tape, and preferred softer tapes, if they equally serve the purpose for diagnosis.

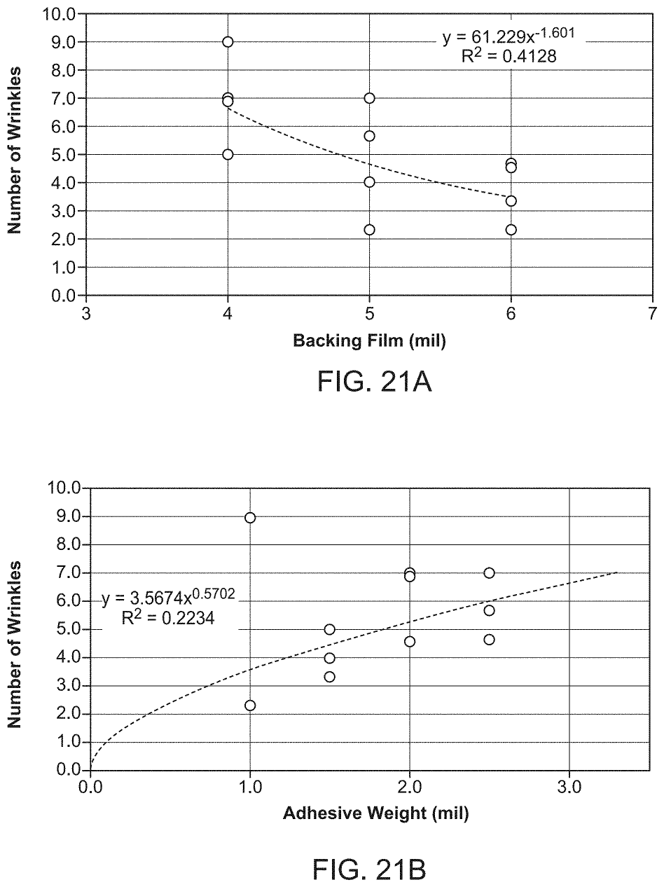

FIG. 21A illustrates a graph of number of wrinkles as a function of backing sheet thickness (mil). FIG. 21B illustrates a graph of number of wrinkles as a function of adhesive layer thickness (mil). FIGS. 21A-21B respectively show that the number of wrinkles formed on the tape is reduced the thicker the backing sheets and the more moderate the adhesive weights.

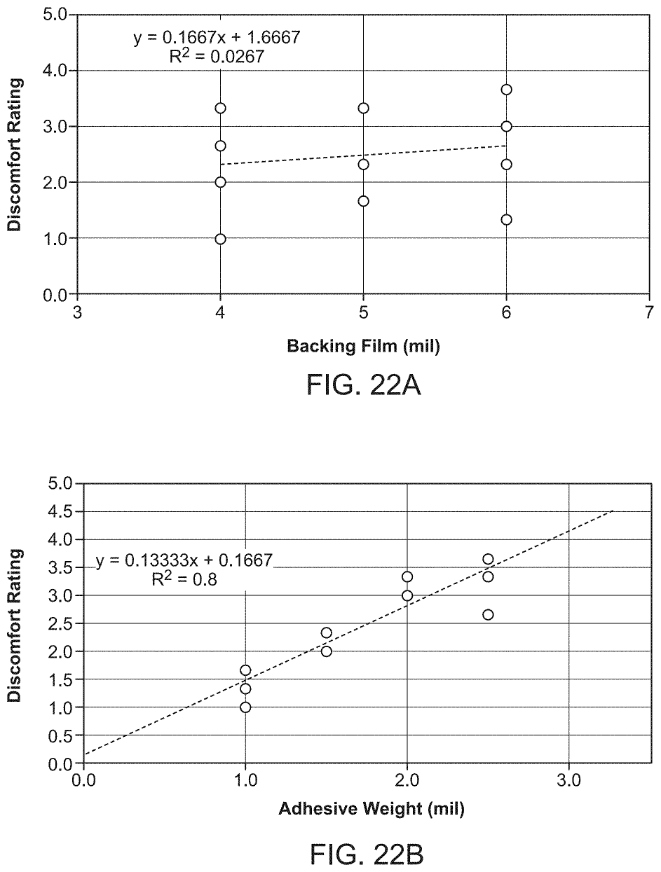

FIG. 22A illustrates a graph of discomfort rating as a function of backing sheet thickness (mil). FIG. 22B illustrates a graph of discomfort rating as a function of adhesive thickness (mil). FIGS. 22A-22B show that there is a stronger correlation between the comfort of peel and the adhesive thickness. Lighter adhesive weights would decrease the cost and the discomfort levels, if they could qualify for the diagnosis processes.

Description of Instrumentation Used—The Labthink© XLW (EC) Auto Tensile Tester was used to determine the tensile, peeling, tearing, heat sealing, adhesive, piercing, opening, low speed unwrapping and pulling force of the plastic film, composite material, flexible packaging material, plastic tube, adhesives, adhesive tapes, medical plasters, release paper, protective films, combination caps, aluminum foils, diaphragm, back sheets, non-woven fabrics, rubber, and paper etc.

### Example 11: Long-Term Outcome of Pigmented Lesions

Summary. The assessment of pigmented lesions suspicious for melanoma remains a challenge. The non-invasive Pigmented Lesion Assay (PLA), which utilizes the adhesive patches described herein, guides biopsy decisions and detects melanoma at its earliest stages based on genomic atypia. The TRUST Study was designed to determine the proportion of true negative lesions among those that initially tested negative. Of the 1781 lesions in the long-term follow-up screening cohort, there were no known melanoma deaths or late-stage melanoma detected. Of the 1233 cases that returned for follow-up evaluation to the clinic, ten lesions received a melanoma diagnosis after the initial PLA test with four (0.3%) at Stage 0 (in situ) and six (0.5%) at Stage 1a. The negative predictive value (NPV) calculated from this subset of 1233 lesions with confirmed follow-up evaluations, but not repeated tested, was 99.2% (CI=98.5-99.6). Of the 302 lesions assessed by means of repeat testing with the PLA, none (0%) were found to have clinically obvious melanoma upon the subject's return to the clinic, confirming the results of the initial chart review. Of these 302 lesions, 88.7% percent (268 lesions) were negative on repeat testing with the PLA and 34 (11.3%) were positive. All 34 lesions (100%) were surgically biopsied, with 3 (1%) diagnosed as Stage 0 (in situ), identified 13, 14 and 19 months after the initial PLA (NPV=99.0% [CI=97.1-99.8]). This long-term repeat-testing study confirmed the NPV of the PLA and found no adverse outcomes related to the test's routine use.

Objective. The objective of the study was to determine the proportion of true negative lesions among those that previously tested negative with the DermTech Pigmented Lesion Assay (PLA).

Methods. Five geographically dispersed trial sites that routinely use the PLA in clinical practice were recruited to participate in this trial. Samples were to be collected from patients who previously had a PLA negative result and retested over an approximate 12- to 24-month period. In addition, patient charts were reviewed over up to a 36-month period to determine:

Did the patient return to the trial site post-PLA?

a. If yes, was the PLA-lesion biopsied

b. If biopsied, was melanoma diagnosed

c. Evidence of mortality

d. Evidence of mortality caused by melanoma

All samples were processed in DermTech's CLIA commercial laboratory located in La Jolla, Calif. The DermTech PLA is a non-invasive adhesive patch test to sample lesions clinically suspicious for melanoma.

The test assesses the expression of two genes associate with melanoma, LINC00518 (long intergenic noncoding RNA 518) and/or PRAME (preferentially expressed antigen in melanoma). The PLA is used to guide biopsy decision and rule out melanoma based on the gene expression results.

Results. The results of the chart review from 2575 lesions are depicted below in Table 22.

TABLE 22

| # of | |
|---|---|
| lesions | Description |
| 2575 | 2575 PLA lesions |
| | ~2400 patients |
| 1781 | Excludes PLA+, QNS, enrolled subjects, |
| | and those not reviewed |
| 1233 | 69.2% returned to the clinic post-PLA |
| 808 | 45.4% returned in the last year |
| 158 | 12.8% hx of melanoma |
| 10 | 0.81% PLA- lesions melanoma+ |
| 2 | Known deaths—neither melanoma related |

Of the reviewed charts, there were 10 PLA-lesions histopathologically assessed as melanoma. Of the 10 melanoma diagnoses 6 (0.5%) were noted to be Stage 1A and 4 (0.3%) melanoma in situ. The time from the PLA-result to the date of melanoma diagnosis ranged from 1 to 33 months after the initial PLA test with an average of 15.1 months (5—less than 12 months, 2—12 to 24 months and 3—greater than 24 months. The negative predictive value calculated from this cohort was 99.2% (CI95%=98.5-99.6) based on the 1233 reviewed charts.

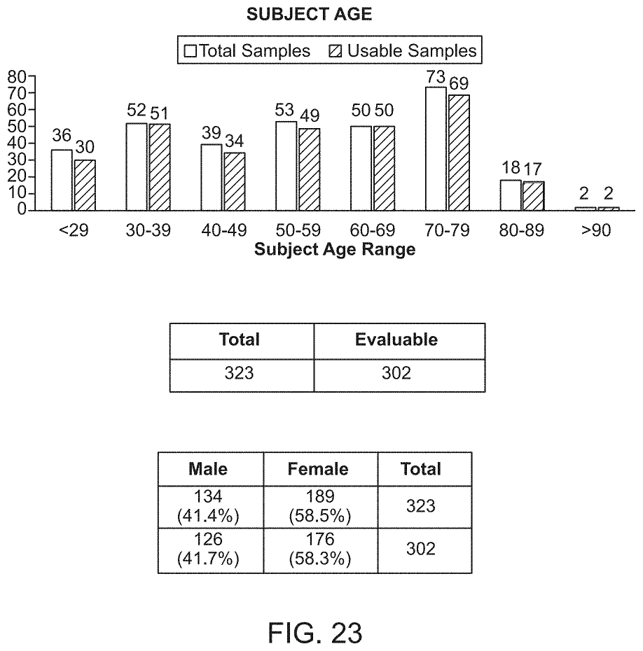

Of the patients who underwent repeat testing of the lesion with the PLA, basic demographic data is presented in FIG. 23.

Enrollment was higher for females versus males with patients aged 70-79 representing the largest age cohort. There was virtually no difference in the sex of patients between those enrolled and those with a repeat PLA result. Data for 21 (6.5%) of the 323 enrolled subjects was not analyzed due to off-label use, and quantity not sufficient for analysis (QNS). PLA positive results for the 302 retested lesions by site are presented below in Table 23.

TABLE 23

| SITE | PRAME+ | LINC+ | DOUBLE+ | TOTAL |
|---|---|---|---|---|
| 01 | 0 | 5 | 1 | 79 |
| 02 | 1 | 0 | 0 | 21 |
| 03 | 0 | 3 | 1 | 19 |
| 04 | 4 | 9 | 7 | 166 |
| 05 | 1 | 2 | 0 | 17 |
| Total | 6 (2.0%) | 19 (6.3%) | 9 (3.0%) | 302 |

Overall LINC+ results were the most frequent positive finding with PRAME+ and DOUBLE+ results occur less frequently. All 34 of these lesions went on to surgical biopsy and those results are presented below in Table 24.

TABLE 24

| PLA+ | PLA+ | Melanoma | Proportion | |
|---|---|---|---|---|
| repeat | surgical | Diagnosis initial | of melanoma | 95% |
| testing | Bx | PLA− | (NPV) | CI |
| 34/302 | 34/34 | 3 | 299/302 | (0.971- |
| (9.9%) | (100%) | (1%) | (99.0%) | 0.998) |

Three Stage 0 (MIS) melanoma were detected in these repeat PLA+ lesions including 1 PRAME+ and 2 DOUBLE+. Repeat testing on these lesions occurred 13, 14 and 19 months after the initial PLA test.

Conclusion. Ten lesions from the screening cohort (1233) received a melanoma diagnosis. Four (0.3%) at Stage 0 (in situ) six (0.5%) at Stage 1a. NPV of the 1233 lesions with confirmed follow-up evaluations was 99.2% (CI95%=98.5-99.6). Of the 323 enrolled subjects, 34 lesions were PLA+ and all went on to surgically biopsied with 3 (1%) diagnosed as Stage 0 (in situ) melanoma. NPV of the 302 lesions was 99.0% (CI95%=97.1-99.8). No adverse outcomes related to the test's routine use.

### Example 12: Melanoma Detection

Tapes T1-T12 of Example 8 are tested for melanoma detection following the general procedure of Example 11.

### Example 13: Tape Concepts for Adhesive Area Increase

As disclose herein, patches may be configured for any color, size and/or shape. In some instances, patches are configured to adhere to specific areas of the body (e.g., face, head, or other area). In some instances, patches are configured as a single sheet covering the entire face. In some instances, multiple patches are configured to sample skin from the face. The shape may be based on a skin collection area. For example, the skin collection device may include a single large patch, include face mask, be shaped for a forehead (e.g., be kidney shaped), be shaped to go under eyes (e.g. crescent), be shaped to cover at least part of a nose, be shaped to cover at least part of a right cheek, be shaped to cover at least part of a left cheek, may be postauricular, may be shaped to cover at least part of a right or left hand, or may be shaped to cover at least part of a right or left foot. Further, a shape may be configured to increase a collection area of the tape.

FIG. 25 shows various tape shapes which may be increase a collection area of a sample. For example, shape 2510 may show a shape of an Applicant comparator device as disclosed herein, e.g., T13 as disclose herein. Shape 2520 may show an area increase of 42.1% over shape 2510. Shape 2530 may show an area increase of 47.8% over 2510. Shape 2540 may show an area increase of 23.1% over 2510. Various shapes used may balance increasing a collection area with providing a portion for handling the collector. In some cases, non-collecting areas may improve patient comfort by providing an area with for example less adhesive material.

### Example 14: Extraction of Proteins

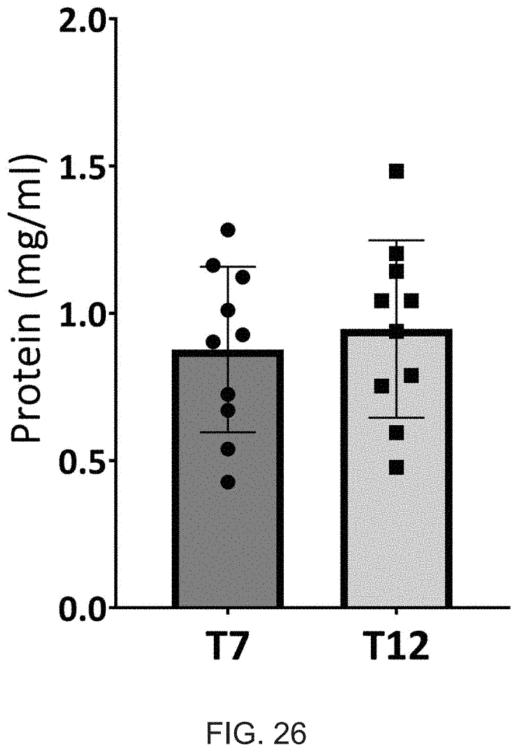

Tapes T7 and T12 from Example 8 were tested for their ability to non-invasively isolate proteins from skin samples. Briefly, samples were lysed in 5 ml tubes with 880 ul of lysis buffer (SK buffer, GenElute, Sigma-Aldrich). The lysate was then loaded onto GenElute kit silica minicolumns and the proteins were extracted following the manufacturer's instructions. Total protein extracted for these two tapes was measured and the results are shown in FIG. 26. Tapes produced over 0.8 mg/mL of protein.

The examples and embodiments described herein are for illustrative purposes only and various modifications or changes suggested to persons skilled in the art are to be included within the spirit and purview of this application and scope of the appended claims.

1. A system for non-invasive collection and analysis of a skin sample, the system comprising:
an adhesive skin sample collection kit comprising at least one adhesive patch, wherein the least one adhesive patch comprises:
a backing layer comprising a collection area;
a non-adhesive handling area; and
an adhesive matrix on a surface of the collection area, wherein the adhesive matrix is configured to adhere an amount of a skin sample.

2. The system of claim 1, wherein one or more of the following:
(a) the backing layer comprises a flexibility to conform to a morphology of a portion of skin, and wherein the backing layer comprises a thickness such the at least one adhesive patch resists wrinkling when the at least one adhesive patch is released from the skin;
(b) the at least one adhesive patch comprises a thickness such that the at least one adhesive patch does not self-adhere when supported by a portion of the non-adhesive handling area with a draft and in multiple orientations;
(c) an amount of extractables and leachables released from the at least one adhesive patch is less about than 3.0 mg/cm² when at least about 25 cm² patch is refluxed for about 3 hours in 80% ethanol;
(d) the at least one adhesive patch comprises a longest dimension of about a wrinkling wavelength of the at least one adhesive patch; and
(e) the adhesive matrix comprises a pressure sensitive adhesive, wherein the pressure sensitive adhesive exhibits a glass transition temperatures lower than 5° C.

3.-4. (canceled)

5. The system of claim 1, wherein the backing layer has an elastic modulus from about 200 to about 2,000 Psi as measured by ASTM D-882

6.-7. (canceled)

8. The system of claim 1, wherein the backing layer has a tensile strength of from about 7 to about 60 MPa.

9.-11. (canceled)

12. The system of claim 1, wherein the backing layer has a thickness greater than about 2 mil as measured by ASTM D6988.

13.-14. (canceled)

15. The system of claim 2, wherein the amount of extractables and leachables released from the at least one adhesive patch is less about than 1.0 mg/cm².

16.-19. (canceled)

20. The system of claim 1, wherein the adhesive matrix comprises a polymer and wherein non-volatile material of the adhesive matrix is comprised on or more monomers of the polymer.

21.-23. (canceled)

24. The system of claim 2, wherein the longest dimension is as less than about 10, about 8, about 6, about 5, about 4, or about 3 cm.

25. (canceled)

26. The system of claim 2, wherein the glass transition temperatures is from about −10 to about −70° C. as measured by ASTM D3418.

27.-35. (canceled)

36. The system of claim 1, wherein the amount of the skin sample is less than about 20 milligrams, less than about 4 milligrams, or from about 1 picogram to about 2000 micrograms of cellular material.

37. (canceled)

38. The system of claim 36, wherein the system comprises a plurality of adhesive patches comprising a total amount of the skin sample, wherein the total amount is less than about 20 milligrams, about 10 milligrams, or about 5 milligrams.

39.-41. (canceled)

42. The system of claim 1, wherein the adhesive matrix comprises a peel adhesion strength from about 1N/inch to about 30N/inch, as measured by ASTM D3330 at a 180° peel adhesion at a pull rates from about 1.0 inch/min to about 12.0 inch/min.

43. (canceled)

44. The system of claim 1, wherein the adhesive matrix comprises one or more of an acrylic, a silicone, and a hydrocarbon rubber.

45. (canceled)

46. The system of claim 44, wherein the hydrocarbon rubber comprises one or more of butyl rubber, styrene-butadiene rubber, ethyl-vinyl acetate polymers, styrene-isoprene-butadiene rubbers, or combinations thereof.

47. (canceled)

48. The system of claim 1, wherein the backing layer comprises a soft, clear, and/or pliable synthetic polymer.

49. The system of claim 48, wherein the synthetic polymer comprises a thermoplastic polyurethane (TPU) or low density polyethylene (LDPE).

50.-55. (canceled)

56. The system of claim 1, wherein the at least one adhesive patch has a haze value less than about 30% as measured by ASTM D1003.

57.-60. (canceled)

61. The system of claim 1, wherein the adhesive matrix comprises at least 12 oz/in² loop tackiness.

62.-76. (canceled)

77. A kit for non-invasive collection and analysis of a skin sample, the kit comprising:
at least one adhesive patch, wherein the least one adhesive patch comprises:
a backing layer comprising a collection area;
a non-adhesive handling area;
an adhesive matrix on a surface of the collection area, wherein the adhesive matrix is configured to adhere to an amount of a skin sample; and
a return or storage receptacle for the at least one adhesive patch

78.-89. (canceled)

90. A method for analyzing a skin sample comprising:
receiving at least one adhesive patch from the system of claim 1; and
quantifying one or more target analytes in the skin sample.
